SEQUENCE LISTING (341 sequences)

SEQ ID NO: 1 - DNA, 10 bp, Artificial Sequence
  Decoy Molecule
gggrnnyycc

SEQ ID NO: 2 - DNA, 10 bp, Artificial Sequence
  Consensus Nucleic Acid Sequence
gggactttcc

SEQ ID NO: 3 - DNA, 5 bp, Artificial Sequence
  Consensus Nucleic Acid Sequence
gggac

SEQ ID NO: 4 - DNA, 18 bp, Artificial Sequence
  Consensus Nucleic Acid Sequence
ctagatttcccgcggatc

SEQ ID NO: 5 - DNA, 10 bp, Artificial Sequence
  Consensus Nucleic Acid Sequence
gggactttcc

SEQ ID NO: 6 - DNA, 10 bp, Artificial Sequence
  Consensus Nucleic Acid Sequence
ccttgaatcc

SEQ ID NO: 7 - DNA, 11 bp, Artificial Sequence
  Consensus Nucleic Acid Sequence
ggggactttcc

SEQ ID NO: 8 - DNA, 4 bp, Artificial Sequence
  Consensus Nucleic Acid Sequence
atgt

SEQ ID NO: 9 - DNA, 12 bp, Artificial Sequence
  Consensus Nucleic Acid Sequence
ggggactttccc

SEQ ID NO: 10 - DNA, 4 bp, Artificial Sequence
  Consensus Nucleic Acid Sequence
tctc

SEQ ID NO: 11 - DNA, 9 bp, Artificial Sequence
  Consensus Nucleic Acid Sequence
gggatttcc

SEQ ID NO: 12 - DNA, 6 bp, Artificial Sequence
  Consensus Nucleic Acid Sequence
ctctgt

SEQ ID NO: 13 - DNA, 9 bp, Artificial Sequence
  Consensus Nucleic Acid Sequence
ggactttcc

SEQ ID NO: 14 - DNA, 6 bp, Artificial Sequence
  Consensus Nucleic Acid Sequence
ctctca

SEQ ID NO: 15 - DNA, 8 bp, Artificial Sequence
  Consensus Nucleic Acid Sequence
gactttcc

SEQ ID NO: 16 - DNA, 4 bp, Artificial Sequence
  Consensus Nucleic Acid Sequence
cttc

SEQ ID NO: 17 - DNA, 9 bp, Artificial Sequence
  Consensus Nucleic Acid Sequence
gactttccc

SEQ ID NO: 18 - DNA, 4 bp, Artificial Sequence
  Consensus Nucleic Acid Sequence
tcca

SEQ ID NO: 19 - DNA, 8 bp, Artificial Sequence
  Consensus Nucleic Acid Sequence
ggatttcc

SEQ ID NO: 20 - DNA, 10 bp, Artificial Sequence
  Consensus Nucleic Acid Sequence
agttgaaggc

SEQ ID NO: 21 - DNA, 9 bp, Artificial Sequence
  Consensus Nucleic Acid Sequence
ggatttccc

SEQ ID NO: 22 - DNA, 8 bp, Artificial Sequence
  Consensus Nucleic Acid Sequence
ttgaaggc

SEQ ID NO: 23 - DNA, 7 bp, Artificial Sequence
  Consensus Nucleic Acid Sequence
gatttcc

SEQ ID NO: 24 - DNA, 8 bp, Artificial Sequence
  Consensus Nucleic Acid Sequence
gatttccc

SEQ ID NO: 25 - DNA, 10 bp, Artificial Sequence
  Consensus Nucleic Acid Sequence
ggactttccc

SEQ ID NO: 26 - DNA, 22 bp, Artificial Sequence
  Consensus Nucleic Acid Sequence
agttgaggggactttcccaggc

SEQ ID NO: 27 - DNA, 15 bp, Artificial Sequence
  Consensus Nucleic Acid Sequence
ctcggactttcctgt

SEQ ID NO: 28 - DNA, 19 bp, Artificial Sequence
  Consensus Nucleic Acid Sequence
agttgagggatttccaggc

SEQ ID NO: 29 - DNA, 20 bp, Artificial Sequence
  Consensus Nucleic Acid Sequence
agttgaggactttcccaggc

SEQ ID NO: 30 - DNA, 19 bp, Artificial Sequence
  Consensus Nucleic Acid Sequence
agttgaggactttccaggc

SEQ ID NO: 31 - DNA, 19 bp, Artificial Sequence
  Consensus Nucleic Acid Sequence
agttgaggatttcccaggc

SEQ ID NO: 32 - DNA, 14 bp, Artificial Sequence
  Consensus Nucleic Acid Sequence
tcggactttccctc

SEQ ID NO: 33 - DNA, 13 bp, Artificial Sequence
  Consensus Nucleic Acid Sequence
atggactttccgt

SEQ ID NO: 34 - DNA, 12 bp, Artificial Sequence
  Consensus Nucleic Acid Sequence
tcggatttcctc

SEQ ID NO: 35 - DNA, 13 bp, Artificial Sequence
  Consensus Nucleic Acid Sequence
tcggactttcctc

SEQ ID NO: 36 - DNA, 17 bp, Artificial Sequence
  Consensus Nucleic Acid Sequence
ttgaggactttccaggc

SEQ ID NO: 37 - DNA, 14 bp, Artificial Sequence
  Consensus Nucleic Acid Sequence
tcgggactttcctc

SEQ ID NO: 38 - DNA, 18 bp, Artificial Sequence
  Consensus Nucleic Acid Sequence
agttgaggatttccaggc

SEQ ID NO: 39 - DNA, 18 bp, Artificial Sequence
  Consensus Nucleic Acid Sequence
tgaggactttccaggctc

SEQ ID NO: 40 - DNA, 16 bp, Artificial Sequence
  Consensus Nucleic Acid Sequence
tgaggactttccaggc

SEQ ID NO: 41 - DNA, 17 bp, Artificial Sequence
  Consensus Nucleic Acid Sequence
ttgcggactttccaggc

SEQ ID NO: 42 - DNA, 14 bp, Artificial Sequence
  Consensus Nucleic Acid Sequence
ctgggactttcctc

SEQ ID NO: 43 - DNA, 18 bp, Artificial Sequence
  Consensus Nucleic Acid Sequence
gttgagggactttccagg

SEQ ID NO: 44 - DNA, 16 bp, Artificial Sequence
  Consensus Nucleic Acid Sequence
ctcgggactttcctgt

SEQ ID NO: 45 - DNA, 16 bp, Artificial Sequence
  Consensus Nucleic Acid Sequence
tcggggactttccctc

SEQ ID NO: 46 - DNA, 19 bp, Artificial Sequence
  Consensus Nucleic Acid Sequence
cagtagtatgtgagcctgc

SEQ ID NO: 47 - DNA, 20 bp, Artificial Sequence
  Consensus Nucleic Acid Sequence
ttgccgtacctgacttagcc

SEQ ID NO: 48 - DNA, 22 bp, Artificial Sequence
  Consensus Nucleic Acid Sequence
agttgaggggactttcccaggc

SEQ ID NO: 49 - DNA, 13 bp, Artificial Sequence
  Consensus Nucleic Acid Sequence
tcgggatttcctc

SEQ ID NO: 50 - DNA, 20 bp, Artificial Sequence
  Consensus Nucleic Acid Sequence
agttgagggactttccaggc

SEQ ID NO: 51 - DNA, 18 bp, Artificial Sequence
  Consensus Nucleic Acid Sequence
agttgagactttccaggc

SEQ ID NO: 52 - DNA, 19 bp, Artificial Sequence
  Consensus Nucleic Acid Sequence
agttgagactttcccaggc

SEQ ID NO: 53 - DNA, 9 bp, Artificial Sequence
  Consensus Nucleic Acid Sequence
ggactttcc

SEQ ID NO: 54 - DNA, 11 bp, Artificial Sequence
  Consensus Nucleic Acid Sequence
aggactttcca

SEQ ID NO: 55 - DNA, 13 bp, Artificial Sequence
  Consensus Nucleic Acid Sequence
ctggactttcctc

SEQ ID NO: 56 - DNA, 17 bp, Artificial Sequence
  Consensus Nucleic Acid Sequence
aagaggactttccagag

SEQ ID NO: 57 - DNA, 17 bp, Artificial Sequence
  Consensus Nucleic Acid Sequence
atatggactttccttaa

SEQ ID NO: 58 - DNA, 17 bp, Artificial Sequence
  Consensus Nucleic Acid Sequence
caacggactttccacac

SEQ ID NO: 59 - DNA, 17 bp, Artificial Sequence
  Consensus Nucleic Acid Sequence
cagtggactttccactg

SEQ ID NO: 60 - DNA, 13 bp, Artificial Sequence
  Consensus Nucleic Acid Sequence
tcgactttccctc

SEQ ID NO: 61 - DNA, 16 bp, Artificial Sequence
  Consensus Nucleic Acid Sequence
ctggggactttccctc

SEQ ID NO: 62 - DNA, 13 bp, Artificial Sequence
  Consensus Nucleic Acid Sequence
tcggatttccctc

SEQ ID NO: 63 - DNA, 11 bp, Artificial Sequence
  Consensus Nucleic Acid Sequence
tcgatttcctc

SEQ ID NO: 64 - DNA, 12 bp, Artificial Sequence
  Consensus Nucleic Acid Sequence
tcgatttccctc

SEQ ID NO: 65 - DNA, 18 bp, Artificial Sequence
  Consensus Nucleic Acid Sequence
ctcggggactttccctca

SEQ ID NO: 66 - DNA, 15 bp, Artificial Sequence
  Consensus Nucleic Acid Sequence
ctcggactttcctca

SEQ ID NO: 67 - DNA, 16 bp, Artificial Sequence
  Consensus Nucleic Acid Sequence
ttgaggatttccaggc

SEQ ID NO: 68 - DNA, 17 bp, Artificial Sequence
  Consensus Nucleic Acid Sequence
ttgaggatttccaggct

SEQ ID NO: 69 - DNA, 18 bp, Artificial Sequence
  Consensus Nucleic Acid Sequence
ttgaggatttccaggctc

SEQ ID NO: 70 - DNA, 15 bp, Artificial Sequence
  Consensus Nucleic Acid Sequence
tgaggactttccagg

SEQ ID NO: 71 - DNA, 13 bp, Artificial Sequence
  Consensus Nucleic Acid Sequence
gaggactttccag

SEQ ID NO: 72 - DNA, 18 bp, Artificial Sequence
  Consensus Nucleic Acid Sequence
gttgaggactttccaggc

SEQ ID NO: 73 - DNA, 15 bp, Artificial Sequence
  Consensus Nucleic Acid Sequence
gaggactttccaggc

SEQ ID NO: 74 - DNA, 14 bp, Artificial Sequence
  Consensus Nucleic Acid Sequence
aggactttccaggc

SEQ ID NO: 75 - DNA, 16 bp, Artificial Sequence
  Consensus Nucleic Acid Sequence
aggactttccaggctc

SEQ ID NO: 76 - DNA, 19 bp, Artificial Sequence
  Consensus Nucleic Acid Sequence
ttgaggactttccaggctc

SEQ ID NO: 77 - DNA, 18 bp, Artificial Sequence
  Consensus Nucleic Acid Sequence
ctcggggactttccctgt

SEQ ID NO: 78 - DNA, 11 bp, Artificial Sequence
  Consensus Nucleic Acid Sequence
aggactttcca

SEQ ID NO: 79 - DNA, 7 bp, Artificial Sequence
  Consensus Nucleic Acid Sequence
ccttgaa

SEQ ID NO: 80 - DNA, 6 bp, Artificial Sequence
  Consensus Nucleic Acid Sequence
agttga

SEQ ID NO: 81 - DNA, 4 bp, Artificial Sequence
  Consensus Nucleic Acid Sequence
ttga

SEQ ID NO: 82 - DNA, 6 bp, Artificial Sequence
  Consensus Nucleic Acid Sequence
agttgc

SEQ ID NO: 83 - DNA, 5 bp, Artificial Sequence
  Consensus Nucleic Acid Sequence
gttga

SEQ ID NO: 84 - DNA, 4 bp, Artificial Sequence
  Consensus Nucleic Acid Sequence
aaga

SEQ ID NO: 85 - DNA, 4 bp, Artificial Sequence
  Consensus Nucleic Acid Sequence
atat

SEQ ID NO: 86 - DNA, 4 bp, Artificial Sequence
  Consensus Nucleic Acid Sequence
caac

SEQ ID NO: 87 - DNA, 4 bp, Artificial Sequence
  Consensus Nucleic Acid Sequence
cagt

SEQ ID NO: 88 - DNA, 4 bp, Artificial Sequence
  Consensus Nucleic Acid Sequence
aggc

SEQ ID NO: 89 - DNA, 4 bp, Artificial Sequence
  Consensus Nucleic Acid Sequence
agag

SEQ ID NO: 90 - DNA, 4 bp, Artificial Sequence
  Consensus Nucleic Acid Sequence
ttaa

SEQ ID NO: 91 - DNA, 4 bp, Artificial Sequence
  Consensus Nucleic Acid Sequence
acac

SEQ ID NO: 92 - DNA, 4 bp, Artificial Sequence
  Consensus Nucleic Acid Sequence
actg

SEQ ID NO: 93 - DNA, 5 bp, Artificial Sequence
  Consensus Nucleic Acid Sequence
aggct

SEQ ID NO: 94 - DNA, 14 bp, Artificial Sequence
  Consensus Nucleic Acid Sequence
ctagatttcccgcg

SEQ ID NO: 95 - DNA, 14 bp, Artificial Sequence
  Consensus Nucleic Acid Sequence
taaagggcgcctag

SEQ ID NO: 96 - DNA, 18 bp, Artificial Sequence
  Consensus Nucleic Acid Sequence
ctagatttcccgcggatc

SEQ ID NO: 97 - DNA, 18 bp, Artificial Sequence
  Consensus Nucleic Acid Sequence
gatctaaagggcgcctag

SEQ ID NO: 98 - DNA, 14 bp, Artificial Sequence
  Consensus Nucleic Acid Sequence
tccagcttcgtagc

SEQ ID NO: 99 - DNA, 13 bp, Artificial Sequence
  Consensus Nucleic Acid Sequence
gaaggatcgatcg

SEQ ID NO: 100 - DNA, 8 bp, Artificial Sequence
  Consensus Nucleic Acid Sequence
tttsgcgs

SEQ ID NO: 101 - DNA, 8 bp, Artificial Sequence
  Consensus Nucleic Acid Sequence
tttggcgc

SEQ ID NO: 102 - DNA, 8 bp, Artificial Sequence
  Consensus Nucleic Acid Sequence
tttcgcgc

SEQ ID NO: 103 - DNA, 8 bp, Artificial Sequence
  Consensus Nucleic Acid Sequence
tttcccgc

SEQ ID NO: 104 - DNA, 8 bp, Artificial Sequence
  Consensus Nucleic Acid Sequence
tttgccgc

SEQ ID NO: 105 - DNA, 8 bp, Artificial Sequence
  Consensus Nucleic Acid Sequence
cttcccgc

SEQ ID NO: 106 - DNA, 8 bp, Artificial Sequence
  Consensus Nucleic Acid Sequence
gttcccgc

SEQ ID NO: 107 - DNA, 8 bp, Artificial Sequence
  Consensus Nucleic Acid Sequence
cttcgcgc

SEQ ID NO: 108 - DNA, 8 bp, Artificial Sequence
  Consensus Nucleic Acid Sequence
ttagcgcc

SEQ ID NO: 109 - DNA, 8 bp, Artificial Sequence
  Consensus Nucleic Acid Sequence
tgagcgcc

SEQ ID NO: 110 - DNA, 8 bp, Artificial Sequence
  Consensus Nucleic Acid Sequence
gtagcgcc

SEQ ID NO: 111 - DNA, 8 bp, Artificial Sequence
  Consensus Nucleic Acid Sequence
ggagcgcc

SEQ ID NO: 112 - DNA, 8 bp, Artificial Sequence
  Consensus Nucleic Acid Sequence
ctagcgcc

SEQ ID NO: 113 - DNA, 8 bp, Artificial Sequence
  Consensus Nucleic Acid Sequence
cgagcgcc

SEQ ID NO: 114 - DNA, 8 bp, Artificial Sequence
  Consensus Nucleic Acid Sequence
gttcgcgc

SEQ ID NO: 115 - DNA, 8 bp, Artificial Sequence
  Consensus Nucleic Acid Sequence
tttgcgcc

SEQ ID NO: 116 - DNA, 8 bp, Artificial Sequence
  Consensus Nucleic Acid Sequence
tgtgcgcc

SEQ ID NO: 117 - DNA, 8 bp, Artificial Sequence
  Consensus Nucleic Acid Sequence
gttgcgcc

SEQ ID NO: 118 - DNA, 8 bp, Artificial Sequence
  Consensus Nucleic Acid Sequence
ggtgcgcc

SEQ ID NO: 119 - DNA, 8 bp, Artificial Sequence
  Consensus Nucleic Acid Sequence
cttgcgcc

SEQ ID NO: 120 - DNA, 8 bp, Artificial Sequence
  Consensus Nucleic Acid Sequence
cgtgcgcc

SEQ ID NO: 121 - DNA, 7 bp, Artificial Sequence
  Consensus Nucleic Acid Sequence
tttccgg

SEQ ID NO: 122 - DNA, 8 bp, Artificial Sequence
  Consensus Nucleic Acid Sequence
tttcgcgg

SEQ ID NO: 123 - DNA, 8 bp, Artificial Sequence
  Consensus Nucleic Acid Sequence
gttggcgc

SEQ ID NO: 124 - DNA, 8 bp, Artificial Sequence
  Consensus Nucleic Acid Sequence
cttggcgc

SEQ ID NO: 125 - DNA, 8 bp, Artificial Sequence
  Consensus Nucleic Acid Sequence
cttgccgc

SEQ ID NO: 126 - DNA, 8 bp, Artificial Sequence
  Consensus Nucleic Acid Sequence
gttgccgc

SEQ ID NO: 127 - DNA, 8 bp, Artificial Sequence
  Consensus Nucleic Acid Sequence
tttggcgg

SEQ ID NO: 128 - DNA, 8 bp, Artificial Sequence
  Consensus Nucleic Acid Sequence
ttaccgcc

SEQ ID NO: 129 - DNA, 8 bp, Artificial Sequence
  Consensus Nucleic Acid Sequence
tgaccgcc

SEQ ID NO: 130 - DNA, 8 bp, Artificial Sequence
  Consensus Nucleic Acid Sequence
gtaccgcc

SEQ ID NO: 131 - DNA, 8 bp, Artificial Sequence
  Consensus Nucleic Acid Sequence
ggaccgcc

SEQ ID NO: 132 - DNA, 8 bp, Artificial Sequence
  Consensus Nucleic Acid Sequence
ctaccgcc

SEQ ID NO: 133 - DNA, 8 bp, Artificial Sequence
  Consensus Nucleic Acid Sequence
cgaccgcc

SEQ ID NO: 134 - DNA, 8 bp, Artificial Sequence
  Consensus Nucleic Acid Sequence
tttccgcc

SEQ ID NO: 135 - DNA, 8 bp, Artificial Sequence
  Consensus Nucleic Acid Sequence
tgtccgcc

SEQ ID NO: 136 - DNA, 8 bp, Artificial Sequence
  Consensus Nucleic Acid Sequence
gttccgcc

SEQ ID NO: 137 - DNA, 8 bp, Artificial Sequence
  Consensus Nucleic Acid Sequence
ggtccgcc

SEQ ID NO: 138 - DNA, 8 bp, Artificial Sequence
  Consensus Nucleic Acid Sequence
cttccgcc

SEQ ID NO: 139 - DNA, 8 bp, Artificial Sequence
  Consensus Nucleic Acid Sequence
cgtccgcc

SEQ ID NO: 140 - DNA, 8 bp, Artificial Sequence
  Consensus Nucleic Acid Sequence
cttcccgg

SEQ ID NO: 141 - DNA, 8 bp, Artificial Sequence
  Consensus Nucleic Acid Sequence
tttgccgg

SEQ ID NO: 142 - DNA, 8 bp, Artificial Sequence
  Consensus Nucleic Acid Sequence
gttcccgg

SEQ ID NO: 143 - DNA, 8 bp, Artificial Sequence
  Consensus Nucleic Acid Sequence
cttcgcgg

SEQ ID NO: 144 - DNA, 8 bp, Artificial Sequence
  Consensus Nucleic Acid Sequence
tttgcgcg

SEQ ID NO: 145 - DNA, 8 bp, Artificial Sequence
  Consensus Nucleic Acid Sequence
ttagcgcg

SEQ ID NO: 146 - DNA, 8 bp, Artificial Sequence
  Consensus Nucleic Acid Sequence
tgtgcgcg

SEQ ID NO: 147 - DNA, 8 bp, Artificial Sequence
  Consensus Nucleic Acid Sequence
tgagcgcg

SEQ ID NO: 148 - DNA, 8 bp, Artificial Sequence
  Consensus Nucleic Acid Sequence
gttgcgcg

SEQ ID NO: 149 - DNA, 8 bp, Artificial Sequence
  Consensus Nucleic Acid Sequence
gtagcgcg

SEQ ID NO: 150 - DNA, 8 bp, Artificial Sequence
  Consensus Nucleic Acid Sequence
ggtgcgcg

SEQ ID NO: 151 - DNA, 12 bp, Artificial Sequence
  Consensus Nucleic Acid Sequence
tttsgcgcgmnr

SEQ ID NO: 152 - DNA, 8 bp, Artificial Sequence
  Consensus Nucleic Acid Sequence
gttggcgg

SEQ ID NO: 153 - DNA, 8 bp, Artificial Sequence
  Consensus Nucleic Acid Sequence
gttcgcgg

SEQ ID NO: 154 - DNA, 8 bp, Artificial Sequence
  Consensus Nucleic Acid Sequence
gttcgcgg

SEQ ID NO: 155 - DNA, 8 bp, Artificial Sequence
  Consensus Nucleic Acid Sequence
tttcccgg

SEQ ID NO: 156 - DNA, 8 bp, Artificial Sequence
  Consensus Nucleic Acid Sequence
cttgccgg

SEQ ID NO: 157 - DNA, 8 bp, Artificial Sequence
  Consensus Nucleic Acid Sequence
gttgccgg

SEQ ID NO: 158 - DNA, 8 bp, Artificial Sequence
  Consensus Nucleic Acid Sequence
tgtcgcgc

SEQ ID NO: 159 - DNA, 8 bp, Artificial Sequence
  Consensus Nucleic Acid Sequence
cttcccgg

SEQ ID NO: 160 - DNA, 8 bp, Artificial Sequence
  Consensus Nucleic Acid Sequence
cgtcgcgc

SEQ ID NO: 161 - DNA, 8 bp, Artificial Sequence
  Consensus Nucleic Acid Sequence
ggtcgcgc

SEQ ID NO: 162 - DNA, 8 bp, Artificial Sequence
  Consensus Nucleic Acid Sequence
tttcgggc

SEQ ID NO: 163 - DNA, 8 bp, Artificial Sequence
  Consensus Nucleic Acid Sequence
tgtggcgc

SEQ ID NO: 164 - DNA, 8 bp, Artificial Sequence
  Consensus Nucleic Acid Sequence
tgtcgcgg

SEQ ID NO: 165 - DNA, 5 bp, Artificial Sequence
  Consensus Nucleic Acid Sequence
acgtg

SEQ ID NO: 166 - DNA, 52 bp, Artificial Sequence
  Consensus Nucleic Acid Sequence
gtgtgctcccagtcagtcaatcctcacgtttatgatggatgaatgaaggcag

SEQ ID NO: 167 - DNA, 49 bp, Artificial Sequence
  Consensus Nucleic Acid Sequence
ttgtgttattagtcaccaacaggcaacgtgcagccggagataaggccag

SEQ ID NO: 168 - DNA, 56 bp, Artificial Sequence
  Consensus Nucleic Acid Sequence
atccccccgcccacagagaggacgtgccacgccagcacgtccgctctccttgccag

SEQ ID NO: 169 - DNA, 57 bp, Artificial Sequence
  Consensus Nucleic Acid Sequence
tgtgctcccagtcagtcaatcctcacgtttatgatggatgaatgaaggcagtcaggt

SEQ ID NO: 170 - DNA, 62 bp, Artificial Sequence
  Consensus Nucleic Acid Sequence
gtgatgaaagagcacaaacgggtgacaaacgtgtctagcgtgattcatcatgaacaggcaca

SEQ ID NO: 171 - DNA, 57 bp, Artificial Sequence
  Consensus Nucleic Acid Sequence
tgcttggtaaactgtaaaatgattagcatacgtgaagcgttagtgtgctccctggca

SEQ ID NO: 172 - DNA, 62 bp, Artificial Sequence
  Consensus Nucleic Acid Sequence
gagcgagccgctgggtgcaggcaggcgacgtgctgccgggctaggctgcccgggggagatga

SEQ ID NO: 173 - DNA, 25 bp, Artificial Sequence
  Consensus Nucleic Acid Sequence
gtggtccgagtcacgtccgaggggg

SEQ ID NO: 174 - DNA, 26 bp, Artificial Sequence
  Consensus Nucleic Acid Sequence
cttcacgtgcggggaccagggaccgt

SEQ ID NO: 175 - DNA, 26 bp, Artificial Sequence
  Consensus Nucleic Acid Sequence
cgcaggcgcaggcggcgcacgtggcc

SEQ ID NO: 176 - DNA, 50 bp, Artificial Sequence
  Consensus Nucleic Acid Sequence
gagtgcgtgcgggactcggagtacgtgacggagccccgagctctcatgcc

SEQ ID NO: 177 - DNA, 53 bp, Artificial Sequence
  Consensus Nucleic Acid Sequence
ggggccccagagcgacgctgagtgcgtgcgggactcggagtacgtgacggagc

SEQ ID NO: 178 - DNA, 52 bp, Artificial Sequence
  Consensus Nucleic Acid Sequence
gcaaggtcgagggccggacgtggggccccagagcgacgctgagtgcgtgcgg

SEQ ID NO: 179 - DNA, 52 bp, Artificial Sequence
  Consensus Nucleic Acid Sequence
ggggcgtgagcggggctgctgcagacgtgcgtgtgggtcatgggggctgctc

SEQ ID NO: 180 - DNA, 43 bp, Artificial Sequence
  Consensus Nucleic Acid Sequence
gccctacgtgctgtctcacacagcctgtctgacctctcgacct

SEQ ID NO: 181 - DNA, 20 bp, Artificial Sequence
  Consensus Nucleic Acid Sequence
ctccggctgcacgttgcctg

SEQ ID NO: 182 - DNA, 55 bp, Artificial Sequence
  Consensus Nucleic Acid Sequence
atggagacataattgaggaacaacgtggaattagtgtcatagcaaatgatctagg

SEQ ID NO: 183 - DNA, 31 bp, Artificial Sequence
  Consensus Nucleic Acid Sequence
gagcggacgtgctggcgtggcacgtcctctc

SEQ ID NO: 184 - DNA, 51 bp, Artificial Sequence
  Consensus Nucleic Acid Sequence
gacgcccgcccccggcccagcctacacgtgggttcccgcacgtccgctggg

SEQ ID NO: 185 - DNA, 51 bp, Artificial Sequence
  Consensus Nucleic Acid Sequence
cgtcagagtgggagcccagcggacgtgcgggaacccacgtgtaggctgggc

SEQ ID NO: 186 - DNA, 30 bp, Artificial Sequence
  Consensus Nucleic Acid Sequence
gtgactacgtgctgcctaggggccactgcc

SEQ ID NO: 187 - DNA, 37 bp, Artificial Sequence
  Consensus Nucleic Acid Sequence
cctgaatgctcttacacacgtacacacacagagcagc

SEQ ID NO: 188 - DNA, 26 bp, Artificial Sequence
  Consensus Nucleic Acid Sequence
ccgggtagctggcgtacgtgctgcag

SEQ ID NO: 189 - DNA, 54 bp, Artificial Sequence
  Consensus Nucleic Acid Sequence
ccttgcggttcgcggcgtgccggacgtgacaaacggaagccgcacgtctcacta

SEQ ID NO: 190 - DNA, 54 bp, Artificial Sequence; identical to SEQ ID NO: 189
  Consensus Nucleic Acid Sequence

SEQ ID NO: 191 - DNA, 20 bp, Artificial Sequence
  Consensus Nucleic Acid Sequence
ctgccagtgcacgtcagtgg

SEQ ID NO: 192 - DNA, 72 bp, Artificial Sequence
  Consensus Nucleic Acid Sequence
gcccggccgctgtcaccgggcaggagagaacgttgcttacgtgcgcccggagtccattggccaaggcgggcc

SEQ ID NO: 193 - DNA, 51 bp, Artificial Sequence
  Consensus Nucleic Acid Sequence
aaggccctgggtccacaggcgtgccgtctgacacgcatcaggcaggcactc

SEQ ID NO: 194 - DNA, 53 bp, Artificial Sequence
  Consensus Nucleic Acid Sequence
ccatttctagggccttgggtccacaggcgtgctggctgacacgcatcaggccg

SEQ ID NO: 195 - DNA, 33 bp, Artificial Sequence
  Consensus Nucleic Acid Sequence
ttcctgcacgtacacacaaagcgcacgtatttc

SEQ ID NO: 196 - DNA, 61 bp, Artificial Sequence
  Consensus Nucleic Acid Sequence
tcagagcacctcgcgagcgtacgtgcctcaggaagtgacgcacagcccccctgggggccgg

SEQ ID NO: 197 - DNA, 65 bp, Artificial Sequence
  Consensus Nucleic Acid Sequence
gggttttgccagactccacagtgcatacgtgggctccaacaggtcctcttccctcccagtcactg

SEQ ID NO: 198 - DNA, 18 bp, Artificial Sequence
  Consensus Nucleic Acid Sequence
gccctacgtgctgtctca

SEQ ID NO: 199 - DNA, 18 bp, Artificial Sequence
  Consensus Nucleic Acid Sequence
tgagacagcacgtagggc

SEQ ID NO: 200 - DNA, 18 bp, Artificial Sequence
  Consensus Nucleic Acid Sequence
ctgtcctccgactgcatg

SEQ ID NO: 201 - DNA, 18 bp, Artificial Sequence
  Consensus Nucleic Acid Sequence
catgcagtcggaggacag

SEQ ID NO: 202 - DNA, 24 bp, Artificial Sequence
  Consensus Nucleic Acid Sequence
cccctcggacgtgactcggaccac

SEQ ID NO: 203 - DNA, 25 bp, Artificial Sequence
  Consensus Nucleic Acid Sequence
gtggtccgagtcacgtccgaggggg

SEQ ID NO: 204 - DNA, 24 bp, Artificial Sequence
  Consensus Nucleic Acid Sequence
tctgtacgtgaccacactcacctc

SEQ ID NO: 205 - DNA, 24 bp, Artificial Sequence
  Consensus Nucleic Acid Sequence
gaggtgagtgtggtcacgtacaga

SEQ ID NO: 206 - DNA, 20 bp, Artificial Sequence
  Consensus Nucleic Acid Sequence
agggccggacgtggggcccc

SEQ ID NO: 207 - DNA, 20 bp, Artificial Sequence
  Consensus Nucleic Acid Sequence
ggggccccacgtccggccct

SEQ ID NO: 208 - DNA, 20 bp, Artificial Sequence
  Consensus Nucleic Acid Sequence
acgctgagtgcgtgcgggac

SEQ ID NO: 209 - DNA, 20 bp, Artificial Sequence
  Consensus Nucleic Acid Sequence
gtcccgcacgcactcagcgt

SEQ ID NO: 210 - DNA, 24 bp, Artificial Sequence
  Consensus Nucleic Acid Sequence
gccctacgtgctgtctcacacagc

SEQ ID NO: 211 - DNA, 24 bp, Artificial Sequence
  Consensus Nucleic Acid Sequence
gctgtgtgagacagcacgtagggc

SEQ ID NO: 212 - DNA, 23 bp, Artificial Sequence
  Consensus Nucleic Acid Sequence
gtgagacgtgcggcttccgtttg

SEQ ID NO: 213 - DNA, 23 bp, Artificial Sequence
  Consensus Nucleic Acid Sequence
caaacggaagccgcacgtctcac

SEQ ID NO: 214 - DNA, 21 bp, Artificial Sequence
  Consensus Nucleic Acid Sequence
ctgccgacgtgcgctccggag

SEQ ID NO: 215 - DNA, 21 bp, Artificial Sequence
  Consensus Nucleic Acid Sequence
ctccggagcgcacgtcggcag

SEQ ID NO: 216 - DNA, 33 bp, Artificial Sequence
  Consensus Nucleic Acid Sequence
gaaatacgtgcgctttgtgtgtacgtgcaggaa

SEQ ID NO: 217 - DNA, 33 bp, Artificial Sequence; identical to SEQ ID NO: 195
  Consensus Nucleic Acid Sequence

SEQ ID NO: 218 - DNA, 21 bp, Artificial Sequence
  Consensus Nucleic Acid Sequence
cgcgagcgtacgtgcctcagg

SEQ ID NO: 219 - DNA, 21 bp, Artificial Sequence
  Consensus Nucleic Acid Sequence
cctgaggcacgtacgctcgcg

SEQ ID NO: 220 - DNA, 21 bp, Artificial Sequence
  Consensus Nucleic Acid Sequence
tgcatacgtgggctccaacag

SEQ ID NO: 221 - DNA, 21 bp, Artificial Sequence
  Consensus Nucleic Acid Sequence
ctgttggagcccacgtatgca

SEQ ID NO: 222 - DNA, 16 bp, Artificial Sequence
  Consensus Nucleic Acid Sequence
aggagacgtgcgagaa

SEQ ID NO: 223 - DNA, 16 bp, Artificial Sequence
  Consensus Nucleic Acid Sequence
ttctcgcacgtctcct

SEQ ID NO: 224 - DNA, 16 bp, Artificial Sequence
  Consensus Nucleic Acid Sequence
aggttacgtgcggaca

SEQ ID NO: 225 - DNA, 16 bp, Artificial Sequence
  Consensus Nucleic Acid Sequence
tgtccgcacgtaacct

SEQ ID NO: 226 - DNA, 16 bp, Artificial Sequence
  Consensus Nucleic Acid Sequence
aggagacgtgctgcct

SEQ ID NO: 227 - DNA, 16 bp, Artificial Sequence
  Consensus Nucleic Acid Sequence
aggcagcacgtctcct

SEQ ID NO: 228 - DNA, 18 bp, Artificial Sequence
  Consensus Nucleic Acid Sequence
tccaatacgtgcagtact

SEQ ID NO: 229 - DNA, 18 bp, Artificial Sequence
  Consensus Nucleic Acid Sequence
agtactgcacgtattgga

SEQ ID NO: 230 - DNA, 18 bp, Artificial Sequence
  Consensus Nucleic Acid Sequence
tccaatgcgtgcagtact

SEQ ID NO: 231 - DNA, 18 bp, Artificial Sequence
  Consensus Nucleic Acid Sequence
agtactgcacgcattgga

SEQ ID NO: 232 - DNA, 18 bp, Artificial Sequence
  Consensus Nucleic Acid Sequence
ggccagacgtgccaccgg

SEQ ID NO: 233 - DNA, 18 bp, Artificial Sequence
  Consensus Nucleic Acid Sequence
ccggtggcacgtctggcc

SEQ ID NO: 234 - DNA, 16 bp, Artificial Sequence
  Consensus Nucleic Acid Sequence
aggcaacgtgcagccg

SEQ ID NO: 235 - DNA, 16 bp, Artificial Sequence
  Consensus Nucleic Acid Sequence
cggctgcacgttgcct

SEQ ID NO: 236 - DNA, 16 bp, Artificial Sequence
  Consensus Nucleic Acid Sequence
aggcaatacgcagccg

SEQ ID NO: 237 - DNA, 16 bp, Artificial Sequence
  Consensus Nucleic Acid Sequence
cggctgcgtattgcct

SEQ ID NO: 238 - DNA, 29 bp, Artificial Sequence
  Consensus Nucleic Acid Sequence
agcggacgtgcagaagttgcacgtcctct

SEQ ID NO: 239 - DNA, 29 bp, Artificial Sequence
  Consensus Nucleic Acid Sequence
agaggacgtgcaacttctgcacgtccgct

SEQ ID NO: 240 - DNA, 18 bp, Artificial Sequence
  Consensus Nucleic Acid Sequence
gtgcatacgtgggctcca

SEQ ID NO: 241 - DNA, 18 bp, Artificial Sequence
  Consensus Nucleic Acid Sequence
tggagcccacgtatgcac

SEQ ID NO: 242 - DNA, 18 bp, Artificial Sequence
  Consensus Nucleic Acid Sequence
gagcgtacgtgcctcagg

SEQ ID NO: 243 - DNA, 18 bp, Artificial Sequence
  Consensus Nucleic Acid Sequence
cctgaggcacgtacgctc

SEQ ID NO: 244 - DNA, 18 bp, Artificial Sequence
  Consensus Nucleic Acid Sequence
ggaacaacgtggaattag

SEQ ID NO: 245 - DNA, 18 bp, Artificial Sequence
  Consensus Nucleic Acid Sequence
ctaattccacgttgttcc

SEQ ID NO: 246 - DNA, 18 bp, Artificial Sequence
  Consensus Nucleic Acid Sequence
gcctacacgtgggttccc

SEQ ID NO: 247 - DNA, 18 bp, Artificial Sequence
  Consensus Nucleic Acid Sequence
gggaacccacgtgtaggc

SEQ ID NO: 248 - DNA, 18 bp, Artificial Sequence
  Consensus Nucleic Acid Sequence
cggagtacgtgacggagc

SEQ ID NO: 249 - DNA, 18 bp, Artificial Sequence
  Consensus Nucleic Acid Sequence
gctccgtcacgtactccg

SEQ ID NO: 250 - DNA, 18 bp, Artificial Sequence
  Consensus Nucleic Acid Sequence
ttgcttacgtgcgcccgg

SEQ ID NO: 251 - DNA, 18 bp, Artificial Sequence
  Consensus Nucleic Acid Sequence
ccgggcgcacgtaagcaa

SEQ ID NO: 252 - DNA, 18 bp, Artificial Sequence
  Consensus Nucleic Acid Sequence
gtgtgtacgtgcaggaaa

SEQ ID NO: 253 - DNA, 18 bp, Artificial Sequence
  Consensus Nucleic Acid Sequence
tttcctgcacgtacacac

SEQ ID NO: 254 - DNA, 27 bp, Artificial Sequence
  Consensus Nucleic Acid Sequence
gcggacgtgcgggaacccacgtgtagg

SEQ ID NO: 255 - DNA, 27 bp, Artificial Sequence
  Consensus Nucleic Acid Sequence
cctacacgtgggttcccgcacgtccgc

SEQ ID NO: 256 - DNA, 16 bp, Artificial Sequence
  Consensus Nucleic Acid Sequence
accgtacgtgctgatc

SEQ ID NO: 257 - DNA, 16 bp, Artificial Sequence
  Consensus Nucleic Acid Sequence
gatcagcacgtacggt

SEQ ID NO: 258 - DNA, 16 bp, Artificial Sequence
  Consensus Nucleic Acid Sequence
ctaatacgtgccgctg

SEQ ID NO: 259 - DNA, 16 bp, Artificial Sequence
  Consensus Nucleic Acid Sequence
cagcggcacgtattag

SEQ ID NO: 260 - DNA, 16 bp, Artificial Sequence
  Consensus Nucleic Acid Sequence
agcagacgtgcaggat

SEQ ID NO: 261 - DNA, 16 bp, Artificial Sequence
  Consensus Nucleic Acid Sequence
atcctgcacgtctgct

SEQ ID NO: 262 - DNA, 16 bp, Artificial Sequence
  Consensus Nucleic Acid Sequence
agcagacgtgcaggca

SEQ ID NO: 263 - DNA, 16 bp, Artificial Sequence
  Consensus Nucleic Acid Sequence
tgcctgcacgtctgct

SEQ ID NO: 264 - DNA, 16 bp, Artificial Sequence
  Consensus Nucleic Acid Sequence
tccgtacgtgctgcac

SEQ ID NO: 265 - DNA, 16 bp, Artificial Sequence
  Consensus Nucleic Acid Sequence
gtgcagcacgtacgga

SEQ ID NO: 266 - DNA, 16 bp, Artificial Sequence
  Consensus Nucleic Acid Sequence
agcagacgtgcagggt

SEQ ID NO: 267 - DNA, 16 bp, Artificial Sequence
  Consensus Nucleic Acid Sequence
accctgcacgtctgct

SEQ ID NO: 268 - DNA, 16 bp, Artificial Sequence
  Consensus Nucleic Acid Sequence
accgtacgtgctgcca

SEQ ID NO: 269 - DNA, 16 bp, Artificial Sequence
  Consensus Nucleic Acid Sequence
tggcagcacgtacggt

SEQ ID NO: 270 - DNA, 16 bp, Artificial Sequence
  Consensus Nucleic Acid Sequence
tccgtacgtgctgcgt

SEQ ID NO: 271 - DNA, 16 bp, Artificial Sequence
  Consensus Nucleic Acid Sequence
acgcagcacgtacgga

SEQ ID NO: 272 - DNA, 16 bp, Artificial Sequence
  Consensus Nucleic Acid Sequence
tgcagacgtgcaggtc

SEQ ID NO: 273 - DNA, 16 bp, Artificial Sequence
  Consensus Nucleic Acid Sequence
gacctgcacgtctgca

SEQ ID NO: 274 - DNA, 16 bp, Artificial Sequence
  Consensus Nucleic Acid Sequence
accgtacgtgctgcta

SEQ ID NO: 275 - DNA, 16 bp, Artificial Sequence
  Consensus Nucleic Acid Sequence
tagcagcacgtacggt

SEQ ID NO: 276 - DNA, 22 bp, Artificial Sequence
  Consensus Nucleic Acid Sequence
ggctgctgcagacgtgcaggtc

SEQ ID NO: 277 - DNA, 22 bp, Artificial Sequence
  Consensus Nucleic Acid Sequence
gacctgcacgtctgcagcagcc

SEQ ID NO: 278 - DNA, 21 bp, Artificial Sequence
  Consensus Nucleic Acid Sequence
ggctgcaggagacgtggagaa

SEQ ID NO: 279 - DNA, 21 bp, Artificial Sequence
  Consensus Nucleic Acid Sequence
ttctccacgtctcctgcagcc

SEQ ID NO: 280 - DNA, 16 bp, Artificial Sequence
  Consensus Nucleic Acid Sequence
agaagacgtgcaggat

SEQ ID NO: 281 - DNA, 16 bp, Artificial Sequence
  Consensus Nucleic Acid Sequence
atcctgcacgtcttct

SEQ ID NO: 282 - DNA, 16 bp, Artificial Sequence
  Consensus Nucleic Acid Sequence
tacagacgtgcaggtc

SEQ ID NO: 283 - DNA, 16 bp, Artificial Sequence
  Consensus Nucleic Acid Sequence
gacctgcacgtctgta

SEQ ID NO: 284 - DNA, 22 bp, Artificial Sequence
  Consensus Nucleic Acid Sequence
ggctgcaccgtacgtgctgatc

SEQ ID NO: 285 - DNA, 22 bp, Artificial Sequence
  Consensus Nucleic Acid Sequence
gatcagcacgtacggtgcagcc

SEQ ID NO: 286 - DNA, 16 bp, Artificial Sequence
  Consensus Nucleic Acid Sequence
tgcatacgtgcaggtc

SEQ ID NO: 287 - DNA, 16 bp, Artificial Sequence
  Consensus Nucleic Acid Sequence
gacctgcacgtatgca

SEQ ID NO: 288 - DNA, 22 bp, Artificial Sequence
  Consensus Nucleic Acid Sequence
ggctgctgcatacgtgcaggtc

SEQ ID NO: 289 - DNA, 22 bp, Artificial Sequence
  Consensus Nucleic Acid Sequence
gacctgcacgtatgcagcagcc

SEQ ID NO: 290 - DNA, 18 bp, Artificial Sequence
  Consensus Nucleic Acid Sequence
gccctacgtgctgtctca

SEQ ID NO: 291 - DNA, 18 bp, Artificial Sequence
  Consensus Nucleic Acid Sequence
ctgtcctccgactgcatg

SEQ ID NO: 292 - DNA, 25 bp, Artificial Sequence
  Consensus Nucleic Acid Sequence
ccccctcggacgtgactcggaccac

SEQ ID NO: 293 - DNA, 24 bp, Artificial Sequence
  Consensus Nucleic Acid Sequence
tctgtacgtgaccacactcacctc

SEQ ID NO: 294 - DNA, 20 bp, Artificial Sequence
  Consensus Nucleic Acid Sequence
agggccggacgtggggcccc

SEQ ID NO: 295 - DNA, 20 bp, Artificial Sequence
  Consensus Nucleic Acid Sequence
acgctgagtgcgtgcgggac

SEQ ID NO: 296 - DNA, 24 bp, Artificial Sequence
  Consensus Nucleic Acid Sequence
gccctacgtgctgtctcacacagc

SEQ ID NO: 297 - DNA, 23 bp, Artificial Sequence
  Consensus Nucleic Acid Sequence
gtgagacgtgcggcttccgtttg

SEQ ID NO: 298 - DNA, 21 bp, Artificial Sequence
  Consensus Nucleic Acid Sequence
ctgccgacgtgcgctccggag

SEQ ID NO: 299 - DNA, 33 bp, Artificial Sequence; identical to SEQ ID NO: 216
  Consensus Nucleic Acid Sequence

SEQ ID NO: 300 - DNA, 21 bp, Artificial Sequence
  Consensus Nucleic Acid Sequence
cgcgagcgtacgtgcctcagg

SEQ ID NO: 301 - DNA, 21 bp, Artificial Sequence
  Consensus Nucleic Acid Sequence
tgcatacgtgggctccaacag

SEQ ID NO: 302 - DNA, 16 bp, Artificial Sequence
  Consensus Nucleic Acid Sequence
aggagacgtgcgagaa

SEQ ID NO: 303 - DNA, 16 bp, Artificial Sequence
  Consensus Nucleic Acid Sequence
aggttacgtgcggaca

SEQ ID NO: 304 - DNA, 16 bp, Artificial Sequence
  Consensus Nucleic Acid Sequence
aggagacgtgctgcct

SEQ ID NO: 305 - DNA, 18 bp, Artificial Sequence
  Consensus Nucleic Acid Sequence
tccaatacgtgcagtact

SEQ ID NO: 306 - DNA, 18 bp, Artificial Sequence
  Consensus Nucleic Acid Sequence
tccaatgcgtgcagtact

SEQ ID NO: 307 - DNA, 18 bp, Artificial Sequence
  Consensus Nucleic Acid Sequence
ggccagacgtgccaccgg

SEQ ID NO: 308 - DNA, 16 bp, Artificial Sequence
  Consensus Nucleic Acid Sequence
aggcaacgtgcagccg

SEQ ID NO: 309 - DNA, 16 bp, Artificial Sequence
  Consensus Nucleic Acid Sequence
aggcaatacgcagccg

SEQ ID NO: 310 - DNA, 29 bp, Artificial Sequence
  Consensus Nucleic Acid Sequence
agcggacgtgcagaagttgcacgtcctct

SEQ ID NO: 311 - DNA, 18 bp, Artificial Sequence
  Consensus Nucleic Acid Sequence
gtgcatacgtgggctcca

SEQ ID NO: 312 - DNA, 18 bp, Artificial Sequence
  Consensus Nucleic Acid Sequence
gagcgtacgtgcctcagg

SEQ ID NO: 313 - DNA, 18 bp, Artificial Sequence
  Consensus Nucleic Acid Sequence
ggaacaacgtggaattag

SEQ ID NO: 314 - DNA, 18 bp, Artificial Sequence
  Consensus Nucleic Acid Sequence
gcctacacgtgggttccc

SEQ ID NO: 315 - DNA, 18 bp, Artificial Sequence
  Consensus Nucleic Acid Sequence
cggagtacgtgacggagc

SEQ ID NO: 316 - DNA, 18 bp, Artificial Sequence
  Consensus Nucleic Acid Sequence
ttgcttacgtgcgcccgg

SEQ ID NO: 317 - DNA, 18 bp, Artificial Sequence
  Consensus Nucleic Acid Sequence
gtgtgtacgtgcaggaaa

SEQ ID NO: 318 - DNA, 27 bp, Artificial Sequence
  Consensus Nucleic Acid Sequence
gcggacgtgcgggaacccacgtgtagg

SEQ ID NO: 319 - DNA, 16 bp, Artificial Sequence
  Consensus Nucleic Acid Sequence
accgtacgtgctgatc

SEQ ID NO: 320 - DNA, 16 bp, Artificial Sequence
  Consensus Nucleic Acid Sequence
ctaatacgtgccgctg

SEQ ID NO: 321 - DNA, 16 bp, Artificial Sequence
  Consensus Nucleic Acid Sequence
agcagacgtgcaggat

SEQ ID NO: 322 - DNA, 16 bp, Artificial Sequence
  Consensus Nucleic Acid Sequence
agcagacgtgcaggca

SEQ ID NO: 323 - DNA, 16 bp, Artificial Sequence
  Consensus Nucleic Acid Sequence
tccgtacgtgctgcac

SEQ ID NO: 324 - DNA, 16 bp, Artificial Sequence
  Consensus Nucleic Acid Sequence
agcagacgtgcagggt

SEQ ID NO: 325 - DNA, 16 bp, Artificial Sequence
  Consensus Nucleic Acid Sequence
accgtacgtgctgcca

SEQ ID NO: 326 - DNA, 16 bp, Artificial Sequence
  Consensus Nucleic Acid Sequence
tccgtacgtgctgcgt

SEQ ID NO: 327 - DNA, 16 bp, Artificial Sequence
  Consensus Nucleic Acid Sequence
tgcagacgtgcaggtc

SEQ ID NO: 328 - DNA, 16 bp, Artificial Sequence
  Consensus Nucleic Acid Sequence
accgtacgtgctgcta

SEQ ID NO: 329 - DNA, 22 bp, Artificial Sequence
  Consensus Nucleic Acid Sequence
ggctgctgcagacgtgcaggtc

SEQ ID NO: 330 - DNA, 21 bp, Artificial Sequence
  Consensus Nucleic Acid Sequence
ggctgcaggagacgtggagaa

SEQ ID NO: 331 - DNA, 16 bp, Artificial Sequence
  Consensus Nucleic Acid Sequence
agaagacgtgcaggat

SEQ ID NO: 332 - DNA, 16 bp, Artificial Sequence
  Consensus Nucleic Acid Sequence
tacagacgtgcaggtc

SEQ ID NO: 333 - DNA, 22 bp, Artificial Sequence
  Consensus Nucleic Acid Sequence
ggctgcaccgtacgtgctgatc

SEQ ID NO: 334 - DNA, 16 bp, Artificial Sequence
  Consensus Nucleic Acid Sequence
tgcatacgtgcaggtc

SEQ ID NO: 335 - DNA, 22 bp, Artificial Sequence
  Consensus Nucleic Acid Sequence
ggctgctgcatacgtgcaggtc

SEQ ID NO: 336 - DNA, 5 bp, Artificial Sequence
  Consensus Nucleic Acid Sequence
acgtg

SEQ ID NO: 337 - DNA, 4 bp, Artificial Sequence
  Consensus Nucleic Acid Sequence
gcgt

SEQ ID NO: 338 - DNA, 18 bp, Artificial Sequence
  Consensus Nucleic Acid Sequence
gccctacgtgctgtctca

SEQ ID NO: 339 - DNA, 18 bp, Artificial Sequence
  Consensus Nucleic Acid Sequence
tgagacagcacgtagggc

SEQ ID NO: 340 - DNA, 21 bp, Artificial Sequence
  Consensus Nucleic Acid Sequence
caccagcgtacgtgcctcagg

SEQ ID NO: 341 - DNA, 21 bp, Artificial Sequence
  Consensus Nucleic Acid Sequence
cctgaggcacgtacgctggtg

(19) United States
(12) Patent Application Publication
(10) Pub. No.: US 2006/0069055 A1
(43) Pub. Date: Mar. 30, 2006

(54) Delivery of polynucleotides

(76) Inventors: Maya Dajee, San Carlos, CA (US); Rolf Ehrhardt, Mill Valley, CA (US); Hans Hofland, Foster City, CA (US); Leslie Mcevoy, Mountain Veiw, CA (US); Tony Marcel Muchamuel, Boulder Creek, CA (US); Brian B. Schryver, Redwood City, CA (US)

Correspondence Address: HELLER EHRMAN LLP, 275 MIDDLEFIELD ROAD, MENLO PARK, CA, 94025-3506, US

(21) Appl. No.: 11/233,511

(22) Filed: Sep. 21, 2005

Related U.S. Application Data
(60) Provisional application No. 60/612,046, filed on Sep. 21, 2004.
(60) Provisional application No. 60/663,497, filed on Mar. 18, 2005.

(51) Int. Cl. A61K 48/00; C12N 15/87

(52) U.S. Cl. 514/44; 435/455

(57) ABSTRACT

The present invention concerns methods and formulations for non-parental delivery of nucleic acid molecules to cells. In particular, the present invention relates to methods and formulations that enhance the transport of poly- and oligonucleotides across biological membranes.

### BACKGROUND OF THE INVENTION

This application claims priority under 35 U.S.C. § 119(e) to provisional application Serial Nos. 60/612,046 filed on Sep. 21, 2004, and 60/663,497 filed on Mar. 18, 2005, the entire disclosures of which is hereby expressly incorporated by reference.

1. Field of the Invention

The present invention concerns methods and formulations for delivering polynucleotides to cells. In particular, the present invention relates to methods and formulations that enhance the transport of polynucleotides across biological membranes.

2. Description of the Related Art

Delivery of Polynucleotides

Advances in the field of biotechnology have led to significant improvements in the treatment of various diseases such as cancer and inflammatory diseases that were previously difficult to treat. Many such advances involve the administration of polynucleotides, including oligonucleotides to a subject, particularly a human subject. The parental administration of such molecules has been shown to be effective for the treatment of a variety diseases and disorders. See, e.g., Draper et al., U.S. Pat. No. 5,595,978, issued Jan. 21, 1997, which discloses intravitreal injection as a means for the direct delivery of antisense oligonucleotides to the vitreous humor of the mammalian eye. See also, Robertson, Nature Biotechnology, 1997, 15, 209, and Genetic Engineering News, 1997, 15, 1, each of which discusses the treatment of Crohn's disease by intravenous infusion of antisense oligonucleotides. Non-parenteral routes (such as transdermal, oral or rectal delivery or other mucosal routes) hold promise for simpler, easier and safer administration of oligonucleotides. For example, transdermal drug delivery of oligonucleotides is an attractive and painless alternative to injections, but due to low skin permeability, only a few transdermal products are available in the market. In order to increase the flux of drugs through the skin, various chemical penetration enhancers have been studied. (See, e.g. Williams et al., Crit. Rev. Ther. Drug Carrier Syst. 9:304-53 (1992); Finnin et al., J. Pharm. Sci. 88: 955-958 (1999); Karande et al., Nature Biotech., 22: 192-197 (2004)). However, one of the problems still remaining for transdermal delivery of oligonucleotides is the fact that at concentrations necessary to induce sufficient penetration enhancement, the formulations used often cause servere irritation to the skin. (See e.g., Lashmar et al., J. Pharm. Pharmacol. 41: 118-122 (1989). Thus, there is a need for topical formulations which sufficiently enhance the skin permeability for delivery of oligonucleotides without causing skin irriation.

Accordingly, there is a need to provide formulations and methods to enhance the availability of novel polynucleotide drugs when administered via non-parenteral routes. It is desirable to develop new formulations and methods that enable the simple, convenient, practical and optimal non-parenteral delivery of polynucleotides, e.g. oligonucleotides.

Transcription Factors

Cells can respond to stimuli, normal or pathological, by changing the levels of expression of specific genes. Therefore, a number of diseases may be linked to an abnormal expression (an overexpression or underexpression) of one or more genes. In general, the expression of these genes is controlled by a variety of transcriptional factors.

Transcription factors represent a group of molecules within the cell that function to connect the pathways from extracellular signals to intracellular responses. Immediately after an environmental stimulus, these proteins which reside predominantly in the cytosol are translocated to the nucleus where they bind to specific DNA sequences in the promoter elements of target genes and activate the transcription of these target genes.

a. NF-κB Transcription Factors

NF-κB is a family of inducible dimeric transcription factors composed of members of the Rel family of DNA-binding proteins that recognize a common sequence motif. In its active DNA-binding form, NF-κB is a heterogeneous collection of dimers, composed of various combinations of members of the NF-κB/Rel family. At present, this family is composed of 5 members, termed p52, p50, p65, cRel and Rel B. The homology between the members of the Rel family is through the Rel homology domain, which is about 300 amino acids in size and constitutes the DNA-binding domain of these proteins.

Different NF-KB dimers exhibit different binding affinities for NF-KB sites bearing the consensus sequence GGGRNNYYCC (SEQ ID NO: 1) where R is purine, Y is pyrimidine and N is any base. The Rel proteins differ in their abilites to activate transcription, such that only p65/RelA and c-Rel were found to contain potent transcriptional- activation domains among the mammalian family members. NF-κB is found in its inactive form in the cytoplasm, where it is bound to the 43-kDa protein IκB that covers the nuclear localization signal region of the p65/p50 dimer. Activation of NF-κB starts with the proteolytic destruction of IκB followed by the transport of the RelA/p50 complex into the nucleus, where it binds to its recognition site on the DNA and activates transcription of target genes. For further review of the NFκB family see, for example, Gomez et al., Frontiers in Bioscience 2:49-60 (1997).

p52 and p50 do not contain transactivation domains. Dimers composed solely of p52 and/or p50 proteins that lack transcriptional activation domains are generally not activators of transcription and can mediate transcriptional repression.

The transcription factors of the Rel/NF-κB family are key regulators of immune and inflammatory responses, and contribute to lymphocyte proliferation, survival and oncogenesis. Thus, NF-κB plays a key role in the expression of several genes involved in the inflammation, cell proliferation and immune responses. (D'Acquisto et al., Gene Therapy 7: 1731-1737 (2000); Griesenbach et al., Gene Therapy 7, 306-313 (2000); Morishita et al., Gene Therapy 7: 1847-1852 (2000)). Among the genes regulated by NF-κB are many which play critical roles in various diseases and conditions, such as rheumatoid arthritis, systemic lupus erythematosus, restenosis, myocardial infarction, ischemia reperfusion injury, glomerulonephritis, atopic dermatitis, saphenous vein graft, Alzheimer's disease, to name a few. See, e.g. Khaled et al. Clinical Immunology and Immunopathology 86(2): 170-179 (1998); Morishita et al., Nature Medicine 3(8): 894-899 (1997); Cho-Chung et al., Current Opinion in Molecular Therapeutics 1(3): 386-392 (1999); Nakamura et al., Gene Therapy 9: 1221-1229 (2002); Shintani et al., Ann. Thorac. Surg. 74: 1132-1138 (2002); and Li et al., J. Neurochem. 74(1): 143-150 (2000).

NF-κB decoys have been proposed for the inhibition of neointimal hyperplasia after angioplasty, restenosis and myocardial infarction (Yoshimura et al., Gene Therapy 8: 1635-1642 (2001); Morishita et al., Nature Medicine 3(8): 894-899 (1997)). The greater inhibition of reperfusion injury, acute, and chronic rejection after transplantation results in a prolongation of allograft survival and decrease in graft coronary artery disease. (Feeley et al., Transplantation 70(11): 1560-1568 (2000)). In vivo transfection of an NFκB decoy provides a novel strategy for treatment of acute myocarditis. (Yokoseki et al., supra). Ueno et al., supra reported that blocking NFκB by NFκB decoy prevented ischemia reperfusion injury in the heart.

It has been shown (Ziegler-Heitbrock et al, J. Leukoc. Biol. 55(10:73-80 (1994); Kastenbauer and Ziegler-Heitbrock, Infect. Immunol. 67(4):1553-9 (1999)) that when a human monocyte cell line, Mono Mac 6, was pre-treated for two days with low doses of lipopolysaccharide (LPS), the response to subsequent LPS stimulation was strongly reduced. Upon stimulation of these LPS-tolerant cells with LPS, these cells exhibit a predominance of the p50 homodimer as shown by the gel shift assay. The authors then tested the effect of the altered NF-κB complexes on gene expression via reporter gene analysis. NF-κB-dependent HIV-1 LTR reporter gene constructs were transfected into Mono Mac 6 cells, followed by pre-culture with and without LPS, and luciferase activity was measured. When LPS-tolerant cells were tested, LPS stimulation did not increase transactivation of the NF-κB-dependent HIV-1 LTR reporter gene. This indicates that the NF-κB complexes present in LPS-tolerant cells are functionally inactive. This also was applicable to the transcription of the NF-κB-controlled TNF gene. Using a TNF promoter-controlled luciferase reporter construct, LPS-tolerant cells showed only a minimal response to LPS stimulation. Therefore, it was concluded that the p50 homodimers induced by LPS tolerance lack transactivation activity. These p50 homodimers instead occupy the cognate NF-κB-binding sites and prevent transactivation and therefore transcription by the p50/p65 complex.

b. E2F Transcription Factors

Another family of transcription factors, the E2F family of transcription factors, plays a pivotal role in the control of cell cycle progression, and regulates the expression of numerous genes, including genes involved in cell cycle regulation, including those encoding c-Myc, c-Myb, Cdc2, proliferating-cell nuclear antigen (PCNA), Cyclin A, dihydrofolate reductase, thymidine kinase, and DNA polymerase α.

E2F is now recognized as a family of six heterodimeric complexes encoded by distinct genes, divided into two distinct groups: E2F proteins (E2F-1-E2F-6) and DP proteins (DP-1 and DP-2). The E2F proteins themselves can be divided into two functional groups, those that induce S-phase progression when over-expressed in quiescent cells (E2Fs 1-3), and those that do not (E2Fs 4-5). E2F-6 is functionally different in that its over-expression has been described to suppress the transactivational effects of co-expression of E2F-1 and DP-1. In addition, it has been reported that E2F-6 expression delays the exit from S-phase rather than inducing S-phase. The proteins from the E2F and DP groups heterodimerize to give rise to E2F activity. All possible combinations of E2F-DP complexes exist in vivo. Individual E2F-DP complexes invoke different transcriptional responses depending on the identity of the E2F moiety and the proteins that are associated with the complex. In addition homodimers of E2F molecules have also been described. (See, e.g., Zheng et al., Genes & Devel 13:666-674 (1999).)

Depending on whether they are associated with the retinoblastoma (Rb) family of pocket proteins, E2F proteins can act either as repressors or as activators of transcription (Hiebert et al. Genes & Devel 6:177-185 (1992); Weintraub et al., Nature 358:259-261 (2002)).

E2F transcription factors are responsible for activating a dozen or more genes that must be turned on during vascular cell growth and multiplication. Its blockade prevents the proliferation of these abnormal cells (neointimal hyperplasia) that eventually result in atherosclerotic lesions. As a result of their biological functions, E2F transcription factors have been implicated in neointimal hyperplasia, neoplasia glomerulonephritis, angiogenesis, and inflammation. Various members of the E2F family have also been described to play a role in cancer, and identified as targets for anti-cancer agents. For an overview of E2F family members, regulation and pathway see, e.g. Harbour, J. W., and Dean, D. C., Genes Dev 14, 2393-2409 (2000); Mundle, S. D., and Saberwal, G., Faseb J 17, 569-574 (2003); and Trimarchi, J. M., and Lees, J. A. Nat Rev Mol Cell Biol 3, 11-20 (2002).

E2F binding sites have been identified in the promoter regions of many cellular genes, and reported, for example, in the following publications: Farnham et al., Biochim. Biophys. Acta 1155:125-131 (1993); Nevins, J. R., Science 258:424-429 (1992); Shan et al., Mol. Cell. Biol. 14:299-309 (1994); Thalmeier et al., Genes Dev. 3:517-536 (1989); Delton et al., EMBO J. 11:1797-1804 (1992); Yamaguchi et al., Jpn. J. Cancer Res. 83:609-617 (1992).

Oligonucleotide decoys targeting E2F transcription factors have been described in PCT Publication No. WO 95/11687, published May 4, 1995, the entire disclosure of which is hereby expressly incorporated by reference.

E2F oligonucleotide decoys are in clinical development as a means of altering the natural history of vein grafts, without the potential hazards of methods that require the introduction of oligonucleotides in vivo, and are expected to be of great clinical value in solving a vexing problem confronting all surgical bypasss and repair of arteries in a variety of clinical circumstances. The U.S. Food and Drug Administration has granted Fast Track designation for an E2F decoy molecule (Corgentech, Inc., South San Francisco, Calif.), which is designed to prevent blocking and failing of vein grafts used in coronary artery and peripheral arterial by-pass procedures.

Further representative references concerning E2F decoy therapy include: Morishita, R., G.H. Gibbons, M. Horiuchi, K.E. Ellison, M. Nakama, L. Zhang, Y. Kaneda, T. Ogihara, and V.J. Dzau. (1995). A gene therapy strategy using a transcription factor decoy of the E2F binding site inhibits smooth muscle proliferation in vivo. Proceedings of the National Academy of Sciences USA, 92, 5855-5859; Dzau, V. J., M. J. Mann, R. Morishita, and Y. Kaneda. (1996). Fusigenic viral liposome for gene therapy in cardiovascular diseases. Proceedings of the National Academy of Sciences USA, 93, 11421-11425; von der Leyen, H. E., M. J. Mann, and V. J. Dzau. (1996). Gene inhibition and gene augmentation for the treatment of vascular proliferative disorders. Semin Interv Cardiology, 1, 209-214; Kaneda, Y., R. Morishita, and V. J. Dzau. (1997). Prevention of restenosis by gene therapy. Annals of the NY Academy of Sciences, 811, 299-308, discussion 308-210; Mann, M. J., and V. J. Dzau. (1997). Genetic manipulation of vein grafts. Current Opinion in Cardiology, 12, 522-527; Mann, M. J., G. H. Gibbons, P. S. Tsao, H. E. von der Leyen, J. P. Cooke, R. Buitrago, R. Kemoff, and V. J. Dzau. (1997). Cell cycle inhibition preserves endothelial function in genetically engineered rabbit vein grafts. Journal of Clinical Investigation, 99, 1295-1301; Morishita, R., G. H. Gibbons, M. Horiuchi, M. Nakajima, K. E. Ellison, W. Lee, Y. Kaneda, T. Ogihara, and V. J. Dzau. (1997). Molecular Delivery System for Antisense Oligonucleotides: Enhanced Effectiveness of Antisense Oligonucleotides by HVJ-liposome Mediated Transfer. Journal of Cardiovascular Pharmacology, 2, 213-222; Braun-Dullaeus, R. C., M. J. Mann, and V. J. Dzau. (1998). Cell cycle progression: new therapeutic target for vascular proliferative disease. Circulation, 98, 82-89; Mann, M. J. (1998). E2F decoy oligonucleotide for genetic engineering of vascular bypass grafts. Antisense Nucleic Acid Drug Development, 8, 171-176; Morishita, R., G. H. Gibbons, M. Horiuchi, Y. Kaneda, T. Ogihara, and V. J. Dzau. (1998). Role of AP-1 complex in angiotensin II-mediated transforming growth factor-beta expression and growth of smooth muscle cells: using decoy approach against AP-1 binding site. Biochemistry and Biophysics Res Community, 243, 361-367; Poston, R. S., K. P. Tran, M. J. Mann, E. G. Hoyt, V. J. Dzau, and R. C. Robbins. (1998). Prevention of ischemically induced neointimal hyperplasia using ex-vivo antisense oligodeoxynucleotides. Journal ofHeart and Lung Transplant, 17, 349-355; Tomita, N., M. Horiuchi, S. Tomita, G. H. Gibbons, J. Y. Kim, D. Baran, and V. J. Dzau. (1998). An oligonucleotide decoy for transcription factor E2F inhibits mesangial cell proliferation in vitro. American Journal of Physiology, 275, F278-284; *Mann, M. J., G. H. Gibbons, H. Hutchinson, R. S. Poston, E. G. Hoyt, R. C. Robbins, and V. J. Dzau. (1999). Pressure-mediated oligonucleotide transfection of rat and human cardiovascular tissues. Proceedings of the National Academy of Sciences USA, 96, 6411-6416; Mann, M. J., A. D. Whittemore, M. C. Donaldson, M. Belkin, M. S. Conte, J. F. Polak, E. J. Orav, A. Ehsan, G. Dell'Acqua, and V. J. Dzau. (1999). Ex-vivo gene therapy of human vascular bypass grafts with E2F decoy: the PREVENT single-centre, randomised, controlled trial. Lancet, 354, 1493-1498; Poston, R. S., M. J. Mann, E. G. Hoyt, M. Ennen, V. J. Dzau, and R. C. Robbins. (1999). Antisense oligodeoxynucleotides prevent acute cardiac allograft rejection via a novel, nontoxic, highly efficient transfection method. Transplantation, 68, 825-832; Tomita, S., N. Tomita, T. Yamada, L. Zhang, Y. Kaneda, R. Morishita, T. Ogihara, V. J. Dzau, and M. Horiuchi. (1999). Transcription factor decoy to study the molecular mechanism of negative regulation of renin gene expression in the liver in vivo. Circulation Research, 84, 1059-1066; von der Leyen, H. E., R. Braun-Dullaeus, M. J. Mann, L. Zhang, J. Niebauer, and V. J. Dzau. (1999). A pressure-mediated nonviral method for efficient arterial gene and oligonucleotide transfer. Human Gene Therapy, 10, 2355-2364; Ehsan, A., and M. J. Mann. (2000). Antisense and gene therapy to prevent restenosis. Vascular Medicine, 5, 103-114; Mann, M. J. (2000). Gene therapy for vein grafts. Current Cardiology Reports, 2, 29-33; Mann, M. J. (2000). Gene therapy for peripheral arterial disease. Molecular Medicine Today, 6, 285-291; Mann, M. J., and V. J. Dzau. (2000). Therapeutic applications of transcription factor decoy oligonucleotides. Journal of Clinical Investigation, 106, 1071-1075; Tomita, N., R. Morishita, S. Tomita, G. H. Gibbons, L. Zhang, M. Horiuchi, Y. Kaneda, J.Kaneda, J. Higaki, T. Ogihara, and V. J. Dzau. (2000). Transcription factor decoy for NFkappaB inhibits TNF-alpha-induced cytokine and adhesion molecule expression in vivo. Gene Therapy, 7, 1326-1332; Ehsan, A., M. J. Mann, G. Dell'Acqua, and V. J. Dzau. (2001). Long-term stabilization of vein graft wall architecture and prolonged resistance to experimental atherosclerosis after E2F decoy oligonucleotide gene therapy. Journal of Thoracic Cardiovascular Surgery, 121,714-722. The complete disclosures of the cited references are hereby expressly incorporated by reference.

c. HIF-1 Transcription Factor

Hypoxia-inducible factor (HIF-1) is a heterodimeric transcription factor that mediates adaptive responses to changes in tissue oxygenation. HIF-1 is a heterodimer that consists of a constitutively expressed HIF-1β subunit and a highly regulated HIF-1α subunit. The synthesis of HIF-1α is oxygen independent; however, the degradation is regulated primarily through oxygen-dependent mechanisms. Activated HIF-1α subunit migrates into the nucleus and dimerizes with the ARNT (aryl receptor nuclear translocator) subunit to form the active transcription factor HIF-1. HIF-1 recognizes the hypoxia-response element (HRE, or 5′-ACGTG-3′ (SEQ ID NO: 1) present in the enhancers or promoters of many genes and leads to their expression. Three subtypes of HIF are currently known (HIF-1, HIF-2, HIF-3); they all affect gene regulation via the conserved HRE.

More than 60 putative direct HIF-1 target genes have been identified based on either the presense of a cis-acting hypoxia response element that contains a HIF-1 binding site, loss of hypoxia-induced expression of the genes HIF-1α-null cells, or increased expression in von Hippel-Lindau (VHL) null cells, or in cells transfected with a HIF-1α expression vector.

Putative HIF-1 regulated genes include adrenomedullin, aldolase A, aldolase C, autocrine motility factor, cathepsin, endocrine gland-derived VEGF, endoglin, endothelin-1, erythropoietin (EPO), fibronectin 1, enolase 1, glucose transporter 1, glucose transporter 3, glyceraldehyde-3-P-dehydrogenase, hexokinase, insulin-like growth-factor 2, insulin-like growth-factor binding protein-1 and 2, keratin 14, 18, and 19, multidrug resistance 1, matrix matalloproteinase 2, nitric oxide synthase 2, plasminogen-activator inhibitor 1, pyruvate kinase M, transforming growth factor-α, transforming growth factor-β2, vascular endothelial growth factor (VEGF), urokinase plasminogen activator receptor, VEGF receptor-2 and vimentin (Semenza, Nature Rev. 3:721-732 (2003)).

Expression of some HIF-1 target genes, such as VEGF, is induced by hypoxia in most cell types, however, for the majority of HIF-1 target genes, expression is induced by hypoxia in a cell-type-specific manner.

HIF-1 activates the transcription of genes that are involved in crucial aspects of cancer biology, including angiogenesis, cell survival, glucose metabolism and invasion. Intratumoral hypoxia and genetic alterations can lead to HIF-1α subunit overexpression, which has been associated with increased patient mortality in several cancer types. HIF-1 and its pathway have been proposed as a target for development of anti-cancer agents (Semenza 2003, supra).

Double-stranded HIF-1 oligodeoxynucleotide decoy (dsODN) molecules have been used to investigate the biological role of HIF-1. HIF-1 dsODN molcules having the following sequences: 5′-GCCCTACGTGCTGTCTCA-3′ (sense) (SEQ ID NO: 338) and 5′-TGAGACAGCACGTAGGGC-3′ (antisense) (SEQ ID NO: 339) were described by Wang and Semenza, J. Biol. Chem. 268:21513-21518 (1993); Wang and Semenza, J. Biol. Chem. 270:1230-1237 (1995). HIF-1 decoy molecules were also disclosed in Oikawa et al., Biochem. Biophys. res. Commun. 289:39-43 (2001); and Yang and Zou, Am. J. Physiol. Renal Physiol. 281 :F900-8 (2001).

E2F dsODN molecules are disclosed in U.S. application publication No. 20050164240 (PCT/IUSO4/33272); NF-κB dsODN molecules are disclosed in U.S. application publication No. 20050182012 (PCT/US04/40673); HIF-1 dsODN molecules are disclosed in PCT/US04/40704, the entire disclosures of which are hereby expressly incorporated by reference.

### SUMMARY OF THE INVENTION

The present invention provides new and efficient methods and formulations for non-parental delivery of nucleic acid molecules, including poly- and oligonucleotides, to cells.

Thus, in one aspect, the present invention concerns a method for delivering a polynucleotide to a cell by contacting a biological membrane with a formulation containing a polynucleotide, at least one penetration enhancer in a total concentration of about 0.2% to about 10% by weight, and alcohol in a concentration of about 1% to 60%.

In one embodiment, the polynucleotide is an oligonucleotide.

In another embodiment, the invention concerns delivering an oligonucleotide to a mammalian cell.

In another embodiment, the invention concerns delivering an oligonucleotide to a human.

In yet another embodiment, the invention concerns delivering an oligonucleotide to a cell wherein the biological membrane is skin or mucosal membrane.

In one embodiment, the penetration enhancer is anionic surfactant. In another embodiment, the anionic surfactant is sodium lauryl sulfate, alkyl ether sulfate or sodium laureth sulfate or N-lauroylsarcosine.

In one embodiment, the penetration enhancer is a non-ionic surfactant. In another embodiment, the non-ionic surfactant is sorbitan monolaurate 20 (Span 20).

In another embodiment, the invention concerns delivering an oligonucleotide to a cell wherein the formulation comprises about 0.4% to about 10% by weight of said penetration enhancer, or about 0.4% to about 1% by weight of said penetration enhancer, or about 0.8% by weight of said penetration enhancer. In one embodiment, the formulation comprises about 0.8% of sodium laureth sulfate. In yet another embodiment, the formulation comprises about 0.6% of N-lauroylsarcosine and about 0.4% of sorbitan monolaurate 20 (Span 20).

In another embodiment, the invention concerns delivering an oligonucleotide to a cell wherein the formulation comprises ethanol. In one embodiment, the formulation comprise about 1% to about 50% by weight of alcohol, or about 5% to about 50% by weight of alcohol, or about 10% to about 50% by weight of alcohol, or about 20% to about 50% by weight of alcohol, or about 30% to about 50% by weight of alcohol, or about 40% to about 50% by weight of alcohol, or about 49% by weight of alcohol, or about 20% by weight of alcohol, or about 10% by weight of alcohol, or about 5% by weight of alcohol, or about 1% by weight of alcohol. In another embodiment, alcohol is ethanol.

In another embodiment, the invention concerns delivering an oligonucleotide to a cell wherein the formulation is an aqueous formulation. In yet another embodiment, the aqueous formulation is aqueous gel-based formulation.

In one embodiment, the aqueous gel-based formulation comprises about 0.8% by weight of sodium laureth sulfate. In another embodiment, the aqueous gel-based formulation further comprises about 1%, about 5%, about 10%, about 20% or about 49% by weight of ethanol.

In another embodiment, the invention concerns delivering an oligonucleotide to a cell wherein the formulation is a liposome-containing formulation.

In another embodiment, the liposome-containing formulation comprises about 0.8% by weight of sodium laureth sulfate. In another embodiment, the liposome-containing formulation further comprises about 2.5%, about 5%, or about 10% by weight of ethanol.

In another embodiment, the liposome-containing formulation comprises about 0.6% by weight of N-lauroylsarcosine, about 0.4% by weight of sorbitan monolaurate 20 (Span 20) and about 5% by weight of ethanol.

In one embodiment, the present invention concerns a method for delivering a polynucleotide to a cell by contacting a biological membrane with an emulsion-based formulation containing a polynucleotide, at least one penetration enhancer in a total concentration of about 0.2% to about 10% by weight, and water. In one embodiment, the polynucleotide is an oligonucleotide.

In one embodiment, the emulsion-based formulation comprises about 0.8% or about 0.35% by weight of sodium laureth sulfate. In another embodiment, the emulsion-based formulation further comprises about 0.15% by weight of 1-phenyl piperazine. In yet another embodiment, the emulsion-based formulation comprises about 0.6% by weight of N-lauroylsarcosine, about 0.4% by weight of sorbitan monolaurate 20 (Span 20) and about 5% by weight of ethanol. In another embodiment, the emulsion-based formulation further comprises about 10% by weight of isopropyl myristate. In yet another embodiment, the emulsion-based formulation further comprises about 10% by weight of glyceryl monostearate.

In one embodiment, the invention concerns delivering a polynucleotide or an oligonucleotide to a cell wherein said cell is vascular smooth muscle cell, tumor cell or endothelial cell.

In another embodiment, the invention concerns delivering an oligonucleotide to a cell wherein the oligonucleotide is a double stranded oligodeoxynucleotide (dsODN) molecule. In one embodiment, the first strand of dsODN molecule is at least partially complementary to the second strand or fully complementary to the second strand. In another embodiment, the dsODN molecule comprises at least one single-stranded overhang. In another embodiment, the dsODN molecule comprises two oligodeoxynucleotide strands that are covalently attached to each other at either the 3′ or the 5′ end, or both, resulting in a dumbbell structure, or a circular molecule. In another embodiment, the dsODN molecule has a phosphodiesterate backbone. In another embodiment, the dsODN molecule has a phosphorothioate backbone. In another embodiment, the dsODN molecule has a mixed phosphodiesterate-phosphorothioate backbone. In another embodiment, the first and second strands of the dsODN molecule are connected to each other solely by Watson-Crick base pairing. In another embodiment, the dsODN is at least 5, 10, 15, 20, or 25 base pairs long.

In yet another embodiment, the invention concerns delivering a dsODN molecule to a cell wherein the dsODN molecule comprises a sequence that is capable of specific binding to a transcription factor. In one embodiment, the transcription factor is selected from the group consisting of E2F, AP-1, AP-2, HIF-1 and NFκB. In another embodiment, the dsODN molecule is capable of specific binding to an NFκB transcription factor.

In a particular embodiment, the dsODN molecules capable of specific binding to an NFκB transcription factor comprises in its first strand, in 5′ to 3′ direction, a sequence of the formula FLANK1-CORE-FLANK2, wherein

CORE is selected from the group consisting of GGGACTTTCC (SEQ ID NO: 5); GGGACTTTCC (SEQ ID NO: 7); GGGACTTTCCC (SEQ ID NO: 9); GGGATTTCC (SEQ ID NO: 11); GGACTTTCC (SEQ ID NO: 13); GACTTTCC (SEQ ID NO: 15); GACTTTCCC (SEQ ID NO: 17); GGATTTCC (SEQ ID NO: 19); GGATTTCCC (SEQ ID NO: 21); GATTTCC (SEQ ID NO: 23); GATTTCCC (SEQ ID NO: 24); GGACTTTCCC (SEQ ID NO: 25); and AGGACTTTCCA (SEQ ID NO: 78);

FLANK1 is selected from the group consisting of CCTTGAA (SEQ ID NO: 79); AT; TC; CTC; CT; AGTTGA (SEQ ID NO: 80), TTGA (SEQ ID NO: 81); AGTTGC (SEQ ID NO: 82); GTTGA (SEQ ID NO: 83); A; AAGA (SEQ ID NO: 84); ATAT (SEQ ID NO: 85); CAAC (SEQ ID NO: 86); CAGT (SEQ ID NO 87); TGA; and GA; and

FLANK2 is selected from the group consisting of TCC; GT; TC; TGT; TCA; TC; CA; AGGC (SEQ ID NO: 88);. AG; AGG; A; AGAG (SEQ ID NO: 89); TTAA (SEQ ID NO: 90); ACAC (SEQ ID NO: 91); ACTG (SEQ ID NO: 92); and AGGCT (SEQ ID NO: 93).

In one embodiment, CORE is selected from the group consisting of GGGATTTCC (SEQ ID NO: 11); GGACTTTCC (SEQ ID NO: 13); and GGATTTCC (SEQ ID NO: 19); and

FLANK1 is AT and FLANK2 is GT; or FLANKI is TC and FLANK2 is TC; or FLANK1 is CTC and FLANK2 is TGT; or FLANKI is AGTTGA (SEQ ID NO: 80) and FLANK 2 is AGGC (SEQ ID NO: 88).

In another embodiment, CORE is GGGATTTCC (SEQ ID NO: 11); or GGACTTTCC (SEQ ID NO: 13), FLANKI is AGTTGA (SEQ ID NO: 80) and FLANK 2 is AGGC (SEQ ID NO: 88).

In yet another embodiment, CORE is GGACTTTCC (SEQ ID NO: 13), FLANK1 is AGTTGA (SEQ ID NO: 80) and FLANK 2 is AGGC (SEQ ID NO: 88).

56In one embodiment, the dsODN molecules capable of specific binding to an NFκB transcription factor include a second strand that is at least partially complementary to said first strand, and may have a phosphodiesterate, phosphorothioate, mixed phosphodiesterate-phosphorothioate, or any other modified backbone. In another embodiment, the two strands may be connected to each other solely by Watson-Crick base pairing and/or by covalent bonds. In yet another embodiment, the dsODN molecules capable of specific binding to an NFκB transcription factor comprises a sequence, in 5′ to 3′ direction, selected from the group consisting of SEQ ID NOs 26 through 77. In yet another embodiment, the dsODN molecules capable of specific binding to an NFκB transcription factor comprises a sequence, in 5′ to 3′ direction, selected from the group consisting of SEQ ID NOs: 26 through 34. In yet another embodiment, the dsODN molecules capable of specific binding to an NFKB transcription factor comprises a sequence, in 5′ to 3′ direction, selected from the group consisting of SEQ ID NOs: 26 through 31. In yet another embodiment, the dsODN molecules capable of specific binding to an NFκB transcription factor comprises the sequence of SEQ ID NO: 30.

In one embodiment, the dsODN molecules capable of specific binding to an NFκB transcription factor is 12 to 28, or 14 to 24, or 14 to 22 base pairs long, and may comprise modified or unusual nucleotides.

In another embodiment, the dsODN molecule is capable of specific binding to an E2F transcription factor. In a particular embodiment, the dsODN molecules capable of specific binding to an E2F transcription factor comprises a core sequence that is capable of specific binding to an E2F transcription factor, flanked by 5′ and 3′ sequences, wherein (i) the core sequence consists of about 5 to 12 base pairs; (ii) the molecule comprises an about 12 to 28 base-pair long double-stranded region composed of two fully complementary strands; and (iii) the E2F dsODN binds to said E2F transcription factor with a binding affinity that is at least about 5-fold of the binding affinity of a deference decoy molecule shown in FIG. 26 (SEQ ID NOS: 94 and 95), as determined by a competitive gel mobility shift binding assay performed on nuclear extract from THP-1 cells.

In another embodiment, the dsODN molecule is capable of specific binding to an HIF-1 transcription factor. In a particular embodiment, the dsODN molecules capable of specific binding to an HIF-1 transcription factor comprises a core sequence that is capable of specific binding to a HIF-1 transcription factor.

In another aspect of the invention, the present invention concerns a formulation containing an oligonucleotide molecule, at least one penetration enhancer in a total concentration of about 0.2% to about 10% by weight, and alcohol in a concentration of about 1% to about 60% by weight.

In one embodiment, the formulation is an aqueous formulation. In another embodiment, the formulation is an aqueous gel-based formulation. In yet another embodiment, the formulation is a liposome-containing formulation.

In another embodiment, the present invention concerns an emulsion formulation containing an oligonucleotide, at least one penetration enhancer in a total concentration of about 0.2% to about 10% by weight, and water.

In all aspects and embodiments, the oligonucleotide preferably is a double-stranded oligodeoxynucleotide (dsODN) molecule, more preferably a transcription factor (TF) dsODN.

The present invention concerns methods and formulations for non-parental delivery of nucleic acid molecules, including poly- and oligonucleotides, to cells. Accordingly, preferred embodiments described herein apply to both the methods and formulations of the present invention.

### BRIEF DESCRIPTION OF THE DRAWINGS

FIG. 1 is a graph showing the effectiveness of treating dustmite antigen induced atopic dermatitis using aqueous gel-based formulation F1 containing NF-κB decoy molecules in a murine model. The aqueous gel-based formulation F1 was comprised of 0.8% sodium laureth sulfate, 49% ethanol, 1.5% HPMC 4000 cps and 48.7% 100 mM phosphate buffer. The skin (ear) thickness of the mouse was measured to quantitate the inflammation and thus the effectiveness of the treatment with NF-κB decoy molecules in various concentrations.

FIG. 2 is a graph showing the effectiveness of treating dustmite antigen induced atopic dermatitis using aqueous gel-based formulation F6 containing NF-κB decoy molecules in a murine model. The skin (ear) thickness of the mouse was measured to quantitate the inflammation and thus the effectiveness of the treatment with NF-κB decoy molecules at 0.25% and 1% concentrations.

FIG. 3 is a graph showing the effectiveness of aqueous gel-based formulations containing NF-κB decoy molecules with various ethanol concentrations.

FIG. 4 is a graph showing the reduction in IL-1β gene expression level in dustmite Ag (Dp) induced contact dermatitis in Nc/Nga mice when treated with aqueous gel-based formulation F2 containing 0.25% NF-κB molecules.

FIG. 5 is a graph showing the reduction in IL-6 gene expression level in dustmite Ag (Dp) induced contact dermatitis in Nc/Nga mice when treated with aqueous gel-based formulation F2 containing 0.25% NF-κB molecules.

FIG. 6 is a graph showing the reduction in TNFα gene expression level in dustmite Ag (Dp) induced contact dermatitis in Nc/Nga mice when treated with aqueous gel-based formulation F2 containing 0.25% NF-κB molecules.

FIG. 7 is a graph showing the reduction in TSLP gene expression level in dustmite Ag (Dp) induced contact dermatitis in Nc/Nga mice when treated with aqueous gel-based formulation F2 containing 0.25% NF-κB molecules.

FIG. 8 shows hematoxylin and eosin staining of formalin-fixed mouse skin with atopic dermatitis that (A) received no treatment, (B) was treated with topical betamethasone, (C) was treated with a formulation containing about 49% ethanol by weight and about 0.8% sodium laureth sulfate by weight and (D) was treated with a formulation containing about 49% ethanol by weight and about 0.8% sodium laureth sulfate by weight containing the NF-κB decoy molecules.

FIG. 9 shows the continuing therapeutic benefit of the NF-κB decoy molecules after the treatment has been terminated and the sudden and severe rebound of swelling and inflammation after the betamethasone treatment was terminated in dustmite Ag (Dp) induced contact dermatitis in Nc/Nga mice.

FIG. 10(A) is a graph showing the thickness of the skin in dustmite Ag (Dp) induced contact dermatitis in Nc/Nga mice without any treatment, when treated with betamethasone and when treated with NF-κB decoy molecules. FIG. 10(A) shows that betamethasone treatment induces skin atrophy.

FIG. 10(B) shows lack of systemic side effects of skin atropy seen with betamethasone treatment in the NF-κB decoy treatment.

FIG. 11 shows the effects of the betamethasone treatment and the NF-κB decoy treatment on the thickness of the skin. FIG. 11 shows that the NF-κB decoy treatment does not cause thinning of the skin.

FIG. 12 shows picro-sirius red staining of formalin-fixed mouse skin with atopic dermatitis that was treated with a formulation F6 containing 0.25% NF-κB decoy molecules and with topical betamethasone.

FIG. 13 shows the delivery of NF-κB decoy molecules to pig skin using the aqueous-gel based formulation F2.

FIG. 14 shows quantitated results from competitive binding gel-shift assay which indicates the presence of NF-κB-bound decoy molecules in the DNFB inflamed pig skin that was treated with aqueous gel-based formulation F2 containing 10% ethanol and varying concentration of NF-κB decoy molecules. The P³²-labeled oligonucleotide probe was radiolabeled in this assay. The amount of band remaining after addition of competitor is graphed. Bands were quantitated using a TYPHOON™ 8600 phosphorimager (Molecular Dynamics, Sunnyvale, Calif.). The presence of NF-κB-bound decoy molecules in the pig skin is determined by the reduction of the binding of the P³²-labeled oligonucleotide probe to the p65 protein on the gel image.

FIG. 15 shows quantitated results from competitive binding gel-shift assaywhich indicates the presence of NF-κB-bound decoy molecules in the DNFB inflamed pig skin that was treated with aqueous gel-based formulation F3 containing 5% ethanol and varying concentration of NF-κB decoy molecules. The P³²-labeled oligonucleotide probe was radiolabeled in this assay. The amount of band remaining after addition of competitor is graphed. Bands were quantitated using a TYPHOON™ 8600 phosphorimager (Molecular Dynamics, Sunnyvale, Calif.). The presence of NF-κB-bound decoy molecules in the pig skin is determined by the reduction of the binding of the P³²-labeled oligonucleotide probe to the p65 protein on the gel image.

FIG. 16 shows quantitated results from competitive binding gel-shift assaywhich indicates the presence of NF-κB-bound decoy molecules in the pig skin that was treated with liposome-containing formulation F9 containing 10% ethanol and varying concentration of NF-κB decoy molecules. The P³²-labeled oligonucleotide probe was radiolabeled in this assay. The amount of band remaining after addition of competitor is graphed. Bands were quantitated using a TYPHOON™ 8600 phosphorimager (Molecular Dynamics, Sunnyvale, Calif.). The presence of NF-κB-bound decoy molecules in the pig skin is determined by the reduction of the binding of the P³²-labeled oligonucleotide probe to the p65 protein on the gel image.

FIG. 17 is a graph showing the IL-6 mRNA expression levels in dinitrofluorobenzene inflamed porcine skin.

FIG. 18 is a graph showing the reduction in relative IL-6 mRNA expression levels in a dinitrofluorobenzene inflamed pig skin treated with liposome-containing formulation F9 with 0.25 and 0.5% of NF-κB decoy molecules when compared to placebo treated or untreated skin.

FIG. 19 is a graph showing the reduction in relative IL-6 mRNA expression levels in a dinitrofluorobenzene inflamed pig skin treated with aqueous gel-based formulation F2 with 0.25% of NF-κB decoy molecules.

FIG. 20 shows the reduction of relative IL-1β mRNA expression levels in dinitrofluorobenzene inflamed porcine skin when treated with aqueous gel-based formulation F10 containing 0.5% or 1% NF-κB decoy molecules.

FIG. 21 shows the results of TUNNEL assay indicating the increased apoptosis due to the betamethasone and NF-κB decoy treatments in Ag (Dp) induced inflammation.

FIG. 22 shows a Ki67 staining of formalin-fixed mouse skin with atopic dermatitis that was treated with aqueous gel-based formulation F6 containing 1% NF-κB decoy molecules and with topical betamethasone.

FIG. 23 is a graph showing the p65/p50 binding of certain NF-κB decoy molecules.

FIG. 24 is a graph showing the p50/p50 binding of certain NF-κB decoy molecules.

FIG. 25 shows quantitated results from EMSA assay. The ability of the decoy molecules designated “E” to compete non-specifically for binding of the transcription factor Oct-1 was tested. The Oct-1 decoy was radiolabeled in this assay. The amount of band remaining after addition of competitor is graphed. Bands were quantitated using a Typhoon Phosphorimager (Molecular Dynamics). The results indicate that the tested NF-κB decoy does not compete non-specifically for a promoter for which it has no specificity. The positive control was cold Oct-1 probe.

FIG. 26 shows the sequences for the “reference decoy molecule” (SEQ ID NOS 94 and 95), “novel decoy molecule” (SEQ ID NOS: 96 and 97) and “scrambled decoy molecule” (SEQ ID NOS 98 and 99), where the core sequences are bolded and underlined.

FIG. 27 shows the results of a competitive binding assay performed with a representative decoy molecule as described in the present invention, in comparison with a reference decoy and a negative control.

FIG. 28 is a matrix that computationally describes the base composition for both the core and the immediate-flanking regions of HIF-1 decoy sequences of the invention.

FIG. 29 shows HIF-1 decoy molecules sorted by their binding affinity, highlighting certain shared sequences correlating with binding affinity.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, NY 1994), and March, Advanced Organic Chemistry Reactions, Mechanisms and Structure 4th ed., John Wiley & Sons (New York, N.Y. 1992), provide one skilled in the art with a general guide to many of the terms used in the present application.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells, and specifically inlcudes oligonucleotides, such as, for example, oligonucleotide decoy molecules and antisense oligonucleotides.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms. Typically, oligonucleotides consist of about 5 to 50, such as 10 to 40, or 15 to 30, nucleotide bases.

The term “antisense oligonucleotide” is used to refer an oligonucleotide or analog thereof that is complementary to a segment of RNA or DNA and that binds to it and inhibits its normal function.

The term “biological membrane” is used to refer to any type of bodily surface of a mammal that acts as a barrier to an external environment. The term specifically includes skin and linings of the body's tubular structure, such as mucosal membrane.

The term “double-stranded” is used to refer to a nucleic acid molecule comprising two complementary nucleotide strands connected to each other by Watson-Crick base pairing. The term specifically includes molecules which, in addition to the double-stranded region formed by the two complementary strands, comprise single-stranded overhang(s), and/or are covalently linked to each other at their 3′ and/or 5′ end(s).

The terms “oligonucleotide decoy,” “double-stranded oligonucleotide decoy,” “oligodeoxynucleotide decoy,” and “double-stranded oligodeoxynucleotide decoy” are used interchangeably, and refer to short nucleic acid molecules comprising a double-stranded region, which bind to and interfere with a biological function of a targeted transcription factor. For example, the terms “NF-κB oligonucleotide decoy,” “double-stranded NF-κB oligonucleotide decoy,” “NF-κB oligodeoxynucleotide decoy,” and “double-stranded NF-κB oligodeoxynucleotide decoy” are used interchangeably, and refer to short nucleic acid molecules comprising a double-stranded region, which bind to and interfere with a biological function of an NF-κB transcription factor. The term “double-stranded” is used to refer to a nucleic acid molecule comprising two complementary nucleotide strands connected to each other by Watson-Crick base pairing. The term specifically includes E2F, NF-κB and HIF-1 oligodeoxynucleotide decoy molecules which, in addition to the double-stranded region formed by the two complementary strands, comprise single-stranded overhang(s). In addition, the term specifically includes E2F, NF-κB and HIF-1 oligodeoxynucleotide decoy molecules in which, in addition to the double-stranded region, the two strands are covalently linked to each other at their 3′ and/or 5′ end(s).

The term “E2F” is used herein in the broadest sense and includes all naturally occurring E2F molecules of any animal, such as mammalian, species, including E2F-1, E2F-2, E2F-3, E2F-4, E2F-5, and E2F-6.

The term “NF-κB” is used herein in the broadest sense and includes all naturally occurring NF-κB molecules of any animal, such as mammalian, species, including all combinations of members of the NF-κB/Rel family, e.g. p52, p50, p65, cRel and Rel B.

The term “HIF-1” is used herein in the broadest sense and includes all naturally occurring HIF-1 molecules of any animal, such as mammalian, species, including the HIF-1α/HIF-1β heterodimer and subunits thereof.

The term “transcription factor binding sequence” is a short nucleotide sequence to which a transcription factor binds. The term specifically includes naturally occurring binding sequences typically found in the regulatory regions of genes the transcription of which is regulated by one or more transcription factors. The term further includes artificial (synthetic) sequences, which do not occur in nature but ate capable of competitively inhibiting the binding of the transcription factor to a binding site in an endogenous gene.

As used herein, the phrase “modified nucleotide” refers to nucleotides or nucleotide triphosphates that differ in composition and/or structure from natural nucleotides and nucleotide triphosphates.

As used herein, the terms “five prime” or “5′” and “three-prime” or “3′” refer to a specific orientation as related to a nucleic acid. Nucleic acids have a distinct chemical orientation such that their two ends are distinguished as either five-prime (5′) or three-prime (3′). The 3′ end of a nucleic acid contains a free hydroxyl group attached to the 3′ carbon of the terminal pentose sugar. The 5′ end of a nucleic acid contains a free hydroxyl or phosphate group attached to the 5′ carbon of the terminal pentose sugar.

As used herein, the term “overhang” refers to a double-stranded nucleic acid molecule, which does not have blunt ends, such that the ends of the two strands are not coextensive, and such that the 5′ end of one strand extends beyond the 3′ end of the opposing complementary strand. It is possible for a linear nucleic acid molecule to have zero, one, or two, 5′ overhangs.

As used herein, the terms “preferential binding,” “preferentially bind” and their grammatical equivalents are used to mean that the specificity/affinity factor is at least about 40, where the specificity/affinity ratio is defined as follows:
Specificy/affinity factor=(S_{p50/p50}−S_{p65/p50)×S}_{p50/p50}/S_{p65/p50}

where S_{p50/p50} equals the molar excess of decoy required to compete 50% of the binding of p50/p50 to the non-mammalian NF-κB promoter from HIV (sequence 113/114) and S_{p65/p50} equals the molar excess of decoy required to compete 50% of the binding of p65/p50 to the non-mammalian NF-κB promoter from HIV (sequence 113/114). The score (S) is assigned as 100 if the decoy is unable to compete at least 50% of the binding at any molar ratio tested.

As used herein, the term “inflammatory disease” or “inflammatory disorder” refers to pathological states resulting in inflammation, typically caused by neutrophil chemotaxis. Examples of such disorders include, without limitation, inflammatory skin diseases including psoriasis and atopic dermatitis; systemic scleroderma and sclerosis; responses associated with inflammatory bowel disease (IBD) (such as Crohn's disease and ulcerative colitis); ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; multiple sclerosis (MS); meningitis; encephalitis; uveitis; osteoarthritis; lupus nephritis; autoimmune diseases such as rheumatoid arthritis (RA), Sjorgen's syndrome, vasculitis; diseases involving leukocyte diapedesis; central nervous system (CNS) inflammatory disorder, multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; etc. The preferred indications include, without limitation, rheumatoid arthritis (RA), rheumatoid spondylitis, gouty arthritis and other arthritic conditions, chronic inflammation, autoimmune diabetes, multiple sclerosis (MS), asthma, systhemic lupus erythrematosus, adult respiratory distress syndrome, Behcet's disease, psoriasis, chronic pulmonary inflammatory disease, graft versus host reaction, Crohn's Disease, ulcerative colitis, inflammatory bowel disease (IBD), Alzheimer's disease, and pyresis, along with any disease or disorder that relates to inflammation and related disorders.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial finction. This activity can be determined and measured, for instance, by cell viability assays, FACS analysis or DNA electrophoresis.

The terms “cancer” and “cancerous” refer to or describe the physiological condition in mammals that is typically characterized by unregulated cell growth. Examples of cancer include, without limitation, carcinoma, lymphoma, leukemia, blastoma, and sarcoma. Specific examples of such cancers include squamous cell carcinoma, small-cell lung cancer, non-small cell lung cancer, breast cancer, pancreatic cancer, glioblastoma multiforme, cervical cancer, stomach cancer, bladder cancer, hepatoma, colon carcinoma, and head and neck cancer. In a preferred embodiment, the cancer includes breast cancer, ovarian cancer, prostate cancer, and lung cancer.

The term “treatment” refers to both therapeutic treatment and prophylactic or preventative measures, wherein the object is to prevent or slow down (lessen) the targeted pathologic condition or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

A “subject” is a vertebrate, preferably a mammal, more preferably a human.

The term “mammal” is used herein to refer to any animal classified as a mammal, including, without limitation, humans, higher primates, rodents, domestic and farm animals, and zoo, sports, or pet animals, such as sheep, dogs, horses, cats, cows, etc. Preferably, the mammal herein is human.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵², Bi²¹², p³² and radioactive isotopes of Lu), chemotherapeutic agents, and toxins such as small molecule toxins or enzymatically active toxins of bacterial, fungal, plant or animal origin, including fragments and/or variants thereof.

The term “chemotherapeutic agent” is used herein to refer to a chemical compound useful in the treatment of cancer. Examples of chemotherapeutic agents include, without limitation, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (1^{I} and calicheamicin 2^{I}₁, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

The term “anti-inflammatory drugs” is used herein includes but not limited to nonsteroidal anti-inflammatory drugs and corticosteroids, such as betamethasone. See, generally, The Merck Manual of Diagnosis and Therapy, 15th Ed., Berkow et al., eds., 1987, Rahway, N.J., pages 2499-2506 and 46-49, respectively.

B. Detailed Description

The present invention concerns methods for delivering polynucleotides, including oligonucleotides, to a cell using formulations that enhance the permeability of the targeted biological membrane and allow the polynucleotide to pass through the biological membrane. These methods and formulations enable the delivery of polynucleotides, including oligonucleotides, for a variety of purposes including, but not limited to, the modulation of gene expression. In particular, the present invention includes formulations which contain the double-stranded oligodeoxynucleotide molecules.

The formulations of the present invention include, but are not limited to, gels, solutions, emulsions, and liposome-containing formulations.

In one aspect of the invention, the formulations include one or more penetration enhancer(s) to facilitate the transport of polynucleotides, including oligonucleotides, across biological membrane into target cells.

Certain embodiments of the invention provide formulations containing one or more polynucleotides in combinations with other pharmaceutically active ingredients, such as, for example, one or more chemotherapeutic agents and/or anti-inflammatory drugs.

In another related embodiment, formulations of the invention may contain one or more oligonucleotides, particularly double-stranded oligodeoxynucleotide decoy molecules, targeted to a transcription factor. Numerous examples of decoy molecules are known in the art.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues.

Penetration Enhancers

The present invention employs penetration enhancers to effect the efficient delivery of polynucleotides, particularly oligonucleotides, such as double-stranded oligodeoxynucleotides, through the skin of animals, such as mammals, including humans. Penetration enhancers are well known in the art and may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers is described below in greater detail.

Surfactants: Surfactants (or “surface-active agents”) are chemical entities which, when dissolved in an aqueous solution, reduce the surface tension of the solution or the interfacial tension between the aqueous solution and another liquid, with the result that absorption of oligonucleotides through the biological membrane is enhanced. In addition to bile salts and fatty acids, such penetration enhancers include, for example, sodium lauryl sulfate, sodium laureth sulfate, N-lauroylsarcosine, sorbitan monolaurate 20 (Span 20), isopropyl myristate, polyoxyethylene-9-lauryl ether and polyoxyethylene-20-cetyl ether (Lee et al., Critical Reviews in Therapeutic Drug Carrier Systems, 1991, p.92); and perfluorochemical emulsions, such as FC-43. (Takahashi et al., J. Pharm. Pharmacol., 1988, 40, 252).

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcamitines, acylcholines, C.sub.1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the biological membrane is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines) (Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the biological membrane (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the formulations of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered oligonucleotides, including glycols such as ethylene glycol and propylene glycol, pyrrols such as 2-pyrrol, azones, and terpenes such as limonene and menthone.

The present invention concerns methods and formulations for delivery of nucleic acid molecules, including polynucleotides and oligonucleotides, to cells comprising contacting the biological membrane with formulations containing at least one penetration enhancer. In one embodiment, the penetration enhancer is an anionic surfactant. In one embodiment, the anionic surfactant is an alkyl sulfate or lauryl sulfate. In another embodiment, the anionic surfactant is an alkyl ether sulfate or sodium laureth sulfate. In one embodiment, the formulation comprises at least two penetration enhancers wherein the penetration enhancers are N-lauroylsarcosine and sorbitan monolaurate 20 (Span 20). In another embodiment, the fomulation further comprises isopropyl myristate.

Carriers

Certain compositions of the present invention also incorporate carrier compounds in the formulation. As used herein, “carrier compound” or “carrier” can, for example, refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a polynucleotide of the present invention and a carrier compound, typically using an excess amount of the latter, can result in a substantial reduction of the amount of the polynucleotide taken up in the liver, kidney or other extracirculatory reservoirs, presumably due to competition between the carrier compound and the nucleic acid for a common receptor. For example, the uptake of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Other Components

The formulations of the present invention may additionally contain other adjunct components conventionally found in pharmaceutical compositions, at their art-established usage levels. Thus, for example, the formulations may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the formulations of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotides of the formulation.

Aqueous Formulations

The formulations of the present invention may be prepared as aqueous gels, aqueous solutions or aqueous suspensions.

In one aspect of the invention, the aqueous formulations of the present invention for delivering polynucleotide to a cell include at least one penetration enhancer in a total concentraion of about 0.2% to about 10% by weight, and alcohol in a concentration of about 1% to about 60% by weight. In one embodiment, the polynucleotide is an oligonucleotide.

In another embodiment, the concentration of the penetration enhancer in the aqueous formulations is about 0.8% by weight. Various penetration enhancers used in the present invention are well known in the art.

In another embodiment, the concentration of the alcohol in the aqueous formulation may range from about 5% to about 50% by weight. In one embodiment, the alcohol is ethanol.

In yet another embodiment, the aqueous formulation is an aqueous gel-based formulation. In one embodiment, the aqueous gel-based formulation comprises about 0.8% by weight of sodium laureth sulfate. In another embodiment, the aqueous gel-based formulation further comprises about 1%, about 5%, about 10%, about 20% or about 49% by weight of ethanol.

In one embodiment, the aqueous gel-based formulations of the present invention are substantially viscous enough to form a viscous gel. In another embodiment, the penetration enhancer in the the aqueous gel-based formulation is sodium laureth sulfate and the alcohol is ethanol. In yet another embodiment, the aqueous gel-based formulation comprises about 0.8% by weight of sodium laureth sulfate and about 49% by weight of ethanol. In another embodiment, the aqueous gel-based formulations may also contain additional pharmaceutically inactive substances, such as hydroxypropylmethylcellulose-4000 (HPMC 4000) and/or magnesium chloride.

In another embodiment, the aqueous gel-based formulation further comprises 1-phenyl piperazine.

The optimal amount of inactive ingredient employed in the aqueous formulations can be conventionally determined based on the particular active pharmaceutical, and the intended use.

Aqueous suspensions may additionally or alternatively contain substances which increase the viscosity of the suspension including, for example, sodium carboxymethylcellulose, sorbitol and/or dextran. The suspension may also contain stabilizers.

Emulsion-Based Formulations

The formulations of the present invention may be prepared as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small ater droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, formulations for delivery of oligonucleotides are formulated as microemulsions.

A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p.245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p.245; Block, in Pharmaceutical Dosage Forms, Lieberman, Rieger and Banker (Eds.), 1988, Marcel Dekker, Inc., New York, N.Y., volume 1, p.335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML3 10), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of cosurfactants and alcohol-free self-emulsifying microemulsion systems are known in the art. In one embodiment of the present invention, the micoremulsion formulations are prepared without alcohol.

The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and triglycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion formulations of the present invention will facilitate the increased systemic absorption of oligonucleotides from the gastrointestinal tract, as well as improve the local cellular uptake of oligonucleotides within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above. Various penetration enhancers used in the present invention are well known in the art.

In one embodiment of the invention, the emulsion-based formulations include one or more penetration enhancer(s). In one embodiment, the penetration enahancers are surfactants. In yet another embodiment, the penetration enhancer(s) may be selected from sodium laureth sulfate, N-laroylsarcosine, sorbitan monolaurate 20 (Span 20) and isopropyl myristate.

The concentration of the penetration enhancer(s) in the emulsion-based formulations may range from about 0.2% to about 10% by weight, or about 0.35% to about 0.8% by weight.

In one embodiment of the present invention, the emulsion-based formulation comprises about 0.35% by weight of sodium laureth sulfate. In another embodiment of the invention, the emulsion-based formulation comprises about 0.8% by weight of sodium laureth sulfate. In yet another embodiment, the emulsion-based formulation comprises about 0.4% by weight of sorbitan monolaurate 20 (Span 20) and about 0.6% by weight of N-laroylsarcosine. In yet another embodiment, the emulsion-based formulation further comprises about 10% by weight of isopropyl myristate.

In another embodiment of the invention, the emulsion-based formulations may also contain additional pharmaceutically inactive substances, such as hydroxypropylmethylcellulose-4000 (HPMC 4000) and preservatives, such as methyl paraben and propyl paraben.

In one embodiment of the invention, the emulsion-based formulation further comprises 1-phenyl piperazine.

Liposome-Containing Formulations

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin. resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising NOVASOME® (glyceryl dilaurate/cholesterol/polyoxyethylene-10-stearyl ether and glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Many liposomes comprising lipids derivatized with one or more hydrophilic polymers, and methods of preparation thereof, are known in the art. Sunamoto et al. (Bull. Chem. Soc. Jpn., 1980, 53, 2768) described liposomes comprising a nonionic detergent that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.). Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersorries are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

In one aspect of the invention, the liposome-containing formulations include one or more penetration enhancer(s) and alcohol. Various penetration enhancers used in the present invention are well known in the art. In one embodiment, the penetration enahancers are surfactants. In yet another embodiment, the penetration enhancer(s) may be selected from sodium laureth sulfate, N-laroylsarcosine, sorbitan monolaurate 20 (Span 20) and phosphatidylcholine (phospholipon 90-H).

In one embodiment, the concentration of the penetration enhancer in the liposome-containing formulation may range from about 0.2% to about 10% by weight, or about 0.4% to about 0.8% by weight.

In another embodiment, the concentration of the alcohol in the liposome-containing formulation may range from about 1% to about 60% by weight, or about 2.5% to about 10% by weight. In one embodiment, the alcohol is ethanol.

In one embodiment of the present invention, the liposome-containing formulation comprises about 2.5% by weight of ethanol. In another embodiment of the present invention, the liposome-containing formulation comprises about 5% by weight of ethanol. In yet another aspect of the invention, the liposome-containing formulation comprises about 10% by weight of ethanol. In yet another embodiment of the invention, the liposome-containing formulation comprises about 0.8% by weight of sodium laureth sulfate and about 2.5%, about 5% or about 10% by weight of ethanol. In one embodiment, the liposome-containing formulation comprises about 0.4% by weight of sorbitan monolaurate 20 (Span 20), about 0.6% by weight of N-laroylsarcosine and about 5% by weight of ethanol. In yet another embodiment, the liposome-containing formulation comprises about 10% by weight of phosphatidylcholine (phospholipon 90-H).

In another aspect of the invention, the liposome-containing formulation further comprises propylene glycol.

In yet another aspect of the invention, the liposome-containing formulations may also contain additional inactive substances, such as buffers, thickeners and preservatives.

Modes of Administration

Administration of the formulations of the present invention maybe topical (including ophthalmic and to mucous membranes including buccal, vaginal and rectal delivery), pulmonary (e.g., by inhalation or insufflation of powders or aerosols, including by nebulizer); intratracheal, intranasal, epidermal, transdermal or oral.

The formulations for topical, oral, parenteral, intrathecal or intraventricular administration may include, but are not limited to, transdermal patches, ointments, lotions, creams, gels, drops, suppositories, sprays, liquids, suspensions or solutions in water or non-aqueous media, capsules, sachets or tablets.

In one embodiment of the invention, a nucleic acid is administered via the rectal mode. In particular, compositions for rectal administration include solutions (enemas) emulsions and suppositories. Rectal suppositories for adults are usually tapered at one or both ends and typically weigh about 2 g each, with infant rectal suppositories typically weighing about one-half as much, when the usual base, cocoa butter, is used (Block, Chapter 87 In: Remington's Pharmaceutical Sciences, 18th Ed., Gennaro, ed., Mack Publishing Co., Easton, Pa., 1990).

The use of absorption-promoting adjuvants is known in the art for the modification of the barrier function of the rectal membrane and has been reviewed (Nishihata and Rytting, Advanced Drug Delivery Reviews, 1997, 28, 205). Absorption-promoting adjuvants have shown promising effects on the performance of formulations of poorly absorbed drugs such as moderately large water-soluble drugs and peptides. Enamine derivatives of amino acids have exhibited absorption promoting properties but the mechanism by which they increase rectal absorption is unclear. Compounds such as chelating agents, and sulfhydryl depleters have been shown to increase the rectal absorption of drugs through the paracellular route as well as the transcellular route. Salicylate and its derivatives also increase absorption of drugs administered via the rectal route via both paracellular and transcellular paths. Fatty acids show properties similar to salicylates when enhancing rectal absorption of drugs. Lectin is also known to increase rectal absorption of drugs via induction of microvillus infusion.

In another embodiment of the invention, one or more nucleic acids are administered via oral delivery.

Compositions for oral administration include powders or granules, suspensions or solutions in water or non-aqueous media, capsules, sachets, troches, tablets or SECs (soft elastic capsules or “caplets”). Thickeners, flavoring agents, diluents, emulsifiers, dispersing aids, carrier substances or binders may be desirably added to such formulations. A tablet may be made by compression or molding, optionally with one or more accessory ingredients. Compressed tablets may be prepared by compressing in a suitable machine, the active ingredients in a free-flowing form such as a powder or granules, optionally mixed with a binder (PVP or gums such as tragecanth, acacia, carrageenan), lubricant (e.g., stearates such as magnesium stearate), glidant (talc, colloidal silica dioxide), inert diluent, preservative, surface active or dispersing agent. Preferred binders/disintegrants include EMDEX (dextrate), PRECIROL (triglyceride), PEG, and AVICEL (cellulose). Molded tablets may be made by molding in a suitable machine a mixture of the powdered compound moistened with an inert liquid diluent. The tablets may optionally be coated or scored and may be formulated so as to provide slow or controlled release of the active ingredients therein.

The use of such formulations has the effect of delivering the nucleic acid to the alimentary canal for exposure to the mucosa thereof. Accordingly, the formulation can contain an enteric material effective in protecting the nucleic acid from pH extremes of the stomach, or in releasing the nucleic acid over time to optimize the delivery thereof to a particular mucosal site. Enteric materials for acid-resistant tablets, capsules and caplets are known in the art and typically include acetate phthalate, propylene glycol, sorbitan monoleate, cellulose acetate phthalate (CAP), cellulose acetate trimellitate and hydroxy propyl methyl cellulose phthalate (HPMCP). Enteric materials may be incorporated within the dosage form or may be a coating substantially covering the entire surface of tablets, capsules or caplets. Enteric materials may also be accompanied by plasticizers which impart flexible resiliency to the material for resisting fracturing, for example during tablet curing or aging. Plasticizers are known in the art and typically include diethyl phthalate (DEP), triacetin, dibutyl sebacate (DBS), dibutyl phthalate (DBP) and triethyl citrate (TEC).

201Various methods for producing formulations for alimentary delivery are well known in the art. See, generally, Naim, Chapter 83; Block, Chapter 87; Rudnic et al., Chapter 89; Porter, Chapter 90; and Longer et al., Chapter 91 In: Remington 's Pharmaceutical Sciences, 18th Ed., Gennaro, ed., Mack Publishing Co., Easton, Pa., 1990. The oligonucleotides described in this invention can be formulated in a known manner into the customary formulations, such as tablets, coated tablets, pills, granules, capsules, aerosols, syrups, gels, emulsions, suspensions and solutions, using inert, non-toxic, pharmaceutically suitable excipients or solvents. The therapeutically active oligonucleotide should in each case be present here in a concentration of about 0.1% to about 0.5% by weight of the total mixture, that is to say in amounts which are sufficient to achieve the stated dosage range. Compositions may be formulated in a conventional manner using additional pharmaceutically acceptable carriers or excipients as appropriate. Thus, the composition may be prepared by conventional means with carriers or excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., starch or sodium starch glycolate); or wetting agents (e.g., sodium lauryl sulfate). Tablets may be coated by methods well known in the art. The preparations may also contain flavoring, coloring and/or sweetening agents as appropriate.

Capsules used for oral delivery may include formulations that are well known in the art. Further, multicompartment hard capsules with control release properties as described by Digenis et al., U.S. Pat. No. 5,672,359, and water permeable capsules with a multi-stage drug delivery system as described by Amidon et al., U.S. Pat. No. 5,674,530 may also be used to formulate the compositions of the present invention.

The formulation of pharmaceutical compositions and their subsequent administration is believed to be within the skill of those in the art.

In general, for therapeutic applications, a patient (i.e., an animal, including a human) having or predisposed to a disease or disorder is administered one or more nucleic acids, including oligonucleotides, in accordance with the invention in a pharmaceutically acceptable carrier in doses ranging from 0.01 μg to 100 g per kg of body weight depending on the age of the patient and the severity of the disorder or disease state being treated. Further, the treatment regimen may last for a period of time which will vary depending upon the nature of the particular disease or disorder, its severity and the overall condition of the patient, and may extend from once daily to once every 20 years. In the context of the invention, the term “treatment regimen” is meant to encompass therapeutic, palliative and prophylactic modalities. Following treatment, the patient is monitored for changes in his/her condition and for alleviation of the symptoms of the disorder or disease state. The dosage of the nucleic acid may either be increased if the patient does not respond significantly to current dosage levels, or the dose may be decreased if an alleviation of the symptoms of the disorder or disease state is observed, or if the disorder or disease state has been abated.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC.sub.50 values found to be effective in in vitro and in vivo animal models. In general, dosage is from 0.01 .mu.g to 100 g per kg of body weight, and may be given once or more daily, weekly, monthly or yearly, or even once every 2 to 20 years. An optimal dosing schedule is used to deliver a therapeutically effective amount of the nucleic acid being administered via a particular mode of administration.

The term “therapeutically effective amount,” for the purposes of the invention, refers to the amount of nucleic acid-containing formulation which is effective to achieve an intended purpose without undesirable side effects (such as toxicity, irritation or allergic response). Although individual needs may vary, determination of optimal ranges for effective amounts of formulations is within the skill of the art. Human doses can be extrapolated from animal studies (Katocs et al., Chapter 27 In: Remington 's Pharmaceutical Sciences, 18th Ed., Gennaro, ed., Mack Publishing Co., Easton, Pa., 1990). Generally, the dosage required to provide an effective amount of a formulation, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s) (Nies et al., Chapter 3 In: Goodman & Gilman 's The Pharmacological Basis of Therapeutics, 9th Ed., Hardman et al., eds., McGraw-Hill, New York, N.Y., 1996).

Following successful treatment, it may be desirable to have the patient undergo maintenance therapy to prevent the recurrence of the disease state, wherein the nucleic acid is administered in maintenance doses, ranging from 0.01 μg to 100 g per kg of body weight, once or more daily, to once every 20 years. For example, in the case of in individual known or suspected of being prone to an autoimmune or inflammatory condition, prophylactic effects may be achieved by administration of preventative doses, ranging from 0.01 μg to 100 g per kg of body weight, once or more daily, to once every 20 years. In like fashion, an individual may be made less susceptible to an inflammatory condition that is expected to occur as a result of some medical treatment, e.g., graft versus host disease resulting from the transplantation of cells, tissue or an organ into the individual.

Formulations for non-parenteral administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic carrier substances suitable for non-parenteral administration which do not deleteriously react with nucleic acids can be used. Suitable pharmaceutically acceptable carriers include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like. The formulations can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously interact with the nucleic acid(s) of the formulation.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

A number of bioequivalents of oligonucleotides and other nucleic acids may also be employed in accordance with the present invention. The invention therefore, also encompasses oligonucleotide and nucleic acid equivalents such as, but not limited to, prodrugs of oligonucleotides and nucleic acids, deletion derivatives, conjugates of oligonucleotides, aptamers, and ribozymes.

The methods and formulations of the present invention also encompass the myriad deletion oligonucleotides, both internal and terminal deletion oligonucleotides, that are synthesized during the process of solid-phase manufacture of oligonucleotides for such deletion sequences are for all practical purposes bioequivalents. Synthetic RNA molecules and their derivatives that possess specific catalytic activities are known as ribozymes and are also considered bioequivalents of oligonucleotides for the purposes of the methods and compositions of the present invention. Also considered bioequivalents of oligonucleotides, for the purposes of the methods and formulations of the present invention, are peptide nucleic acids (PNAs) and aptamers (see, generally, Ellington et al., Nature, 1990, 346, 818; U.S. Pat. No.5,523,389 (Ecker et al., Jun. 4, 1996)).

The name aptamer has been coined by Ellington and Szostak (Nature, 1990, 346, 818) for nucleic acid molecules that fit and therefore bind with significant specificity to non-nucleic acid ligands such as peptides, proteins and small molecules such as drugs and dyes. Because of these specific ligand binding properties, nucleic acids and oligonucleotides that may be classified as aptamers may be readily purified or isolated via affinity chromatography using columns that bear immobilized ligand. Aptamers may be nucleic acids that are relatively short to those that are as large as a few hundred nucleotides. For example, Ellington and Szostak have reported the discovery of RNA aptamers that are 155 nucleotides long and that bind dyes such as Cibacron Blue and Reactive Blue 4 (Ellington and Szostak, Nature, 1990, 346, 818) with very good selectivity. While RNA molecules were first referred to as aptamers, the term as used in the present invention refers to any nucleic acid or oligonucleotide that exhibits specific binding to small molecule ligands including, but not limited to, DNA, RNA, DNA derivatives and conjugates, RNA derivatives and conjugates, modified oligonucleotides, chimeric oligonucleotides, and gapmers.

In one embodiment, the present invention is drawn to the non-parenteral administration of a nucleic acid, such as an oligonucleotide, having biological activity, to an animal. By “having biological activity,” it is meant that the nucleic acid functions to modulate the expression of one or more genes in an animal as reflected in either absolute function of the gene (such as ribozyme activity) or by production of proteins coded by such genes. In the context of this invention, “to modulate” means to either effect an increase (stimulate) or a decrease (inhibit) in the expression of a gene. Such modulation can be achieved by, for example, a double-stranded oligodeoxynucleotide decoy molecule which binds to a target transcription factor by a variety of mechanisms known in the art. Various double-stranded oligodeoxynucleotide decoy molecules administerd using the formulations of the present invention are described below in greater detail.

In an animal other than a human, the formulations and methods of the invention can be used to study the function of one or more genes in the animal.

As stated, the formulations and methods of the invention are useful therapeutically, i.e., to provide therapeutic, palliative or prophylactic relief to an animal, including a human, having or suspected of having or of being susceptible to, a disease or disorder that is treatable in whole or in part with one or more nucleic acids. The term “disease or disorder” (1) includes any abnormal condition of an organism or part, especially as a consequence of infection, inherent weakness, environmental stress, that impairs normal physiological functioning; (2) excludes pregnancy per se but not autoimmune and other diseases associated with pregnancy; and (3) includes cancers and tumors. The term “having or suspected of having or of being susceptible to” indicates that the subject animal has been determined to be, or is suspected of being, at increased risk, relative to the general population of such animals, of developing a particular disease or disorder as herein defined. For example, a subject animal could have a personal and/or family medical history that includes frequent occurrences of a particular disease or disorder. As another example, a subject animal could have had such a susceptibility determined by genetic screening according to techniques known in the art (see, e.g., U.S. Congress, Office of Technology Assessment, Chapter 5 In: Genetic Monitoring and Screening in the Workplace, OTA-BA-455, U.S. Government Printing Office, Washington, D.C., 1990, pages 75-99). The term “a disease or disorder that is treatable in whole or in part with one or more nucleic acids” refers to a disease or disorder, as herein defined, (1) the management, modulation or treatment thereof, and/or (2) therapeutic, palliative and/or prophylactic relief therefrom, can be provided via the administration of more nucleic acids. In a preferred embodiment, such a disease or disorder is treatable in whole or in part with a double-stranded oligodeoxynucleotide decoy molecule.

Oligonucleotide decoys molecules targeting various transcription factors which can be administered using one or more of the formulations described above, include but are not limited to, those described below.

NF-κB Oligonucleotide Decoy Molecule

One aspect of the invention is an idea that by designing decoy molecules which could bind p65/p50 and/or cRel/p50 heterodimers and not p50/p50 homodimers, or which would preferentially bind p65/p50 and/or cRel/p50 heterodimers, one could provide extra blockade of NF-κB driven promoters by leaving p50/p50 homodimers behind to occupy these sites. As a result, such selective decoy molecules have the potential to block NF-κB activity than NF-κB decoys known in the art.

Design of NF-κB Decoys with Improved Properties

1. Design of NF-κB dsODN Molecules

The oligonucleotide decoys of the present invention have been designed taking advantage of the crystal structure of the p50/p65 heterodimer bound to the immunoglobulin light-chain gene (Chen et al, Nature 391(6665):410-3 (1998)) which contains the consensus sequence of 5′-GGGACTTTCC-3′ (SEQ ID NO: 2). The authors showed that p50 contacts the 5-base-pair subsite 5′-GGGAC-3′ (SEQ ID NO: 3) and that p65 contacts the 4-base-pair subsite 5′TTCC-3′ (SEQ ID NO: 4). The DNA contacts by the p50/p65 heterodimer are similar to those in the homodimer structures (Ghosh et al, Nature 373(6512):303-10 (1995); Muller et al, FEBS Lett. 369(1):113-7 (1995)).

In one embodiment, the NF-κB dsODN molecules of the present invention consist of two oligonucleotide strands which are attached to each other by Watson-Crick base pairing. While typically all nucleotides in the two strands participate in the base pairing, this is not a requirement. Oligonucleotide decoy molecules, where one or more, such as 1-3 or 1 or 2 nucleotides are not involved in base pairing are also included. In addition, the double stranded decoys may contain 3′ and/or 5′ single stranded overhangs.

In another embodiment, the NF-κB dsODN molecules of the present invention comprise two oligonucleotide strands which are attached to each other by Watson-Crick base pairing, and are additionally covalently attached to each other at either the 3′ or the 5′ end, or both, resulting in a dumbell structure, or a circular molecule. The covalent linkage may be provided, for example, by phosphodiester linkages or other linking groups, such as, for example, phosphothioate, phosphodithioate, or phosphoarnidate linkages.

Generally, the dsODN molecules of the invention comprise a core sequence that is capable of specific binding to an NF-κB transcription factor, flanked by 5′ and/or 3′ sequences, wherein the core sequence consists of about 5 to 14, or about 6 to 12. or about 7 to about 10 base pairs; and the flanking sequences are about 2 to 8, or about 2 to 6, or about 2 to 4 base pairs long. The molecule typically comprises an about 12 to 28, preferably about 14 to 24 base-pair long double-stranded region composed of two fully or partially complementary strands (including the core and flaking sequences).

Changing the core sequence (including its length, sequence, base modifications and backbone structure) it is possible to change the binding affinity, the stability and the specificity of the NF-κB decoy molecule. Indeed, the NF-κB dsODN molecules of the present invention, which bind the p65/p50 and/or cRel/p50 heterodimers with high affinity and exhibit no or only low affinity binding for the p50/p50 homodimers, were designed by deleting or changing targeted residues in the binding site (core) of a consensus oligonucleotide decoy, based on the crystal structure of the p65/p50 heterodimer binding to DNA.

In addition, changes in the flanking sequence have a genuine impact on and can significantly increase the in vivo stability of the NF-κB decoy molecule, and may affect binding affinity and/or specificity. In particular, the shape/structure of the NF-κB decoy molecule can be changed by changing the sequences flaking the core binding sequence, which can result in improved stability and/or binding affinity. The shape and structure of the DNA are influenced by the base pair sequence, length of the DNA, backbone and nature of the nucleotide (i.e. native DNA vs. modified sugars or bases). Thus, the shape and/or structure of the molecule can also be changed by other approaches, such as, for example, by changing the total length, the length of the fully complementary, double-stranded region within the molecule, by alterations within the core and flanking sequences, by changing the backbone structure and by base modifications.

The nucleotide sequences present in the decoy molecules of the present invention may comprise modified or unusual nucleotides, and may have alternative backbone chemistries. Synthetic nucleotides may be modified in a variety of ways, see, e.g. Bielinska et al. Science 250:997-1000 (1990). Thus, oxygens may be substituted with nitrogen, sulfur or carbon; phosphorus substituted with carbon; deoxyribose substituted with other sugars, or individual bases substituted with an unnatural base. Thus replacement of non-bridging oxygen atoms of the internucleotide linkage with a sulfur group (to yield a phosphorothioate linkage) has been useful in increasing the nuclease resistance of the dsODN molecule. Experiments determining the relationship between the number of sulfur modifications and stability and specificity of the NF-κB dsODN molecules herein are set forth in the Example below.

In each case, any change will be evaluated as to the effect of the modification on the binding ability and affinity of the oligonucleotide decoy to the NF-κB transcription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g., Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β,D-mannosylqueosine, 5-methoxycarbonylmethyl-2-thiouridine, 5-metoxycarbonalmethyluridine, 5-methoxyuridine, 2-methylthio-N6-isopentenyladenosine, N-((9-beta-D-ribofuransyl-2-methylthiopurine-6-yl)carbamoyl)threonine, N-((9-beta-D-ribofuranosylpurine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuranosylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl. Other likages, such as phosphothioate, phosphodithioate, etc. are also possible.

The decoys of the present invention can also contain modified or analogous forms of the ribose or deoxyribose sugars generally present in polynucleotide structures. Such modifications include, without limitation, 2′-substituted sugars, such as 2′-O-methyl-, 2′-O-allyl, 2′-fluoro- and 2′azido-ribose, carboxylic sugar analogs, α-anomeric sugars, epimeric sugars, such as arabinose, xyloses, lyxoses, pyranose sugars, furanose sugars, sedoheptuloses, acyclic analogs and abasic nucleoside analogs, such as methyl riboside.

In general, the oligonucleotide decoys of the present invention are preferably comprised of greater than about 50%, more preferably greater than about 80%, most preferably greater than about 90% conventional deoxyribose nucleotides.

The NF-κB dsODN decoys of the present invention can be further modified to facilitate their localization, purification, or improve certain properties thereof. For example, a nuclear localization signal (NLS) can be attached to the decoy molecules, in order to improve their delivery to the cell nucleus. The NF-κB/Rel proteins include a common Rel homology domain, which encompasses the NLS. In a preferred embodiment such naturally occurring NLS, or a variant thereof, is used in the decoy molecules of the present invention.

In addition, the NF-κB decoy molecules of the invention may be conjugated with carrier molecules, such as peptides, proteins or other types of molecules, as described, for example, in the following references: Avrameas et al., J. Autoimmun 16, 383-391 (2001); Avrameas et al., Bioconjug. Chem. 10: 87-93 (1999); Gallazzi et al., Bioconjug. Chem. 14, 1083-1095 (2003); Ritter, W. et al., J. Mol. Med. 81, 708-717 (2003).

The NF-κB decoy molecules of the invention may further be derivatized to include delivery vehicles which improve delivery, distribution, target specific cell types or facilitate transit through cellular barriers. Such delivery vehicles include, without limitation, cell penetration enhancers, liposomes, lipofectin, dendrimers, DNA intercalators, and nanoparticles.

2. Synthesis of NF-κB dsODN Molecules

The NF-κB sdODN decoy molecules of the present invention can be synthesized by standard phosphodiester or phosphoramidate chemistry, using commercially available automatic synthesizers. The specific dsODN molecules described in the example have been synthesized using an automated DNA synthesizer (Model 380B; Applied Biosystems, Inc., Foster City, Calif.). The decoys were purified by column chromatography, lyophilized, and dissolved in culture medium. Concentrations of each decoy were determined spectrophotometrically.

3. Characterization of NF-κB dsODN Molecules

The NF-κB decoy molecules of the present invention can be conveniently tested and characterized in a gel shift, or electrophoretic mobility shift (EMSA) assay. This assay provides a rapid and sensitive method for detecting the binding of transcription factors to DNA. The assay is based on the observation that complexes of protein and DNA migrate through a non-denaturing polyacryamide gel more slowly than free double-stranded oligonucleotides. The gel shift assay is performed by incubating a purified protein, or a complex mixture of proteins (such as nuclear extracts), with a ³²p end-labeled DNA fragment containing a transcription factor-binding site. The reaction products are then analyzed on a non-denaturing polyacrylamide gel. The specificity of the transcription factor for the binding site is established by competition experiments, using excess amounts of oligonucleotides either containing a binding site for the protein of interest or a scrambled DNA sequence. The identity of proteins contained within a complex is established by using an antibody which recognizes the protein and then looking for either reduced mobility of the DNA-protein-antibody complex or disruption of the binding of this complex to the radiolabeled oligonucleotide probe.

In designing the selective NF-κB decoys herein, based on the crystal structure of p65/p50 heterodimer binding to DNA, targeted residues in the binding site (core) of the consensus oligonucleotide decoy were deleted. The ultimate goal was to design a double-stranded oligonucleotide which was able to bind p65/p50 and/or cRel/p50 heterodimers, preferably both the p65/p50 and cRel/p50 heterodimers, with high affinity and exhibited low affinity for p50/p50 homodimers. To achieve this aim, a variety of NF-κB decoys were tested for their ability to bind the different NF-κB proteins in a gel shift assay as described in the following Example.

4. Use of NF-κB dsODN Molecules

NF-κB is involved in the regulation of the transcription of numerous genes. A representative grouping and listing of genes transcriptionally activated by NF-κB is provided below.

Cytokines/chemokines and their modulators, such as, for example, interferon-γ (IFN-γ), interferon-β (IFN-β), interleukins, such as, IL-1, I1-2, IL-6, IL-8, IL-9, IL-10, IL-11, IL-12, IL-13, IL-15, lymphotoxin-α, lymphotoxin-β, TNF-α, MIP-1, MIP-2, MIP-3, RANTES, TNF-α, TRAIL.

Immunoregulators, such as, for example, BRL-1, CCR5, CCR7, CD137, CD154, CD40 and CD40 ligand, CD48, CD83, CD23, IL-2 receptor a chain, certain immunoglobulin heavy and light chains, MHC Class I antigen, T cell receptor subunits, TNF-receptor (p75/80).

Proteins involved in antigen presentation, such as, for example, Complement B, Complement component 3, TAPI, and tapasin.

Cell adhesion molecules, such as, for example, E- and P-selectin, ICAM-1, MadCAM-1, VCAM-1, and Tenascin-C.

Acute phase proteins, such as, for example, angiotensinogen, β-defensin-2, complement factors, tissue factor-1 (TF-1), urokinase-type plasminogen activator.

Stress response genes, such as, for example, angiotensin-2, COX-2, MAP4K1, Phospholipase A2.

Cell surface receptors, such as, for example, CD23, CD69, EGF-R, Lox-1, Mdr1.

Regulators of apoptosis, such as, for example, Bfl1, Bc1-xL, Caspase-11, CD95 (Fas), TRAF-1, TRAF-2.

Growth factors and their modulators, such as, for example, G-CSF, GM-CSF, EPO, IGFBP-1, IGFBP-2, M-CSF, VEGF-C.

Early response genes, such as, for example, TIEG, B94, Egr-1.

In addition, NF-κB regulates the transcription of other transcription factors, such as c-myc-, c-myb, A20, junB, p53, WT1, and viruses.

Thus, inhibition of NF-κB induced expression of proinflammatory cytokines, such as IL-1 and TNF-α, and immune modulators, is useful in the treatment of inflammatory, immune and autoimmune diseases, such as rheumatoid arthritis (RA) (Roshak et al., Current Opinion in Pharmacology 2:316-321 (2002)); Crohn's disease and inflammatory bowel disease (IBD) (Dijkstra et al., Scandinavian J. of Gastroenterology Suppl. 236:37-41 (2002)), pancreatitis (Eeber and Adler, Pancreatology 1:356-362 (2001)), periodonitis (Nichols et al., Annals of Periodontology 6:20-29 (2001)); lupus (Kammer and Tsokos, Current Directions in Autoimmunity 5:131-150 (2002)); asthma (Pahl and Szelenyi, Inflammation Research 51:273-282 (2002)); and ocular allergy (Bielory et al., Opinion in Allergy and Clinical Immunology 2:435-445 (2003)).

Since NF-κB plays a pivotal role in the coordinated transactivation of cytokine and adhesion molecule genes involved in atherosclerosis and lesion formation after vascular injury (Yoshimura et al., Gene Therapy 8: 1635-1642 (2001)); neuronal damage after cerebral ischemia (Ueno et al., J. Thoracic and Cardiovascular Surgery 122(4): 720-727 (2001)); chronic airway inflammation (Griesenbach et al., Gene Therapy 7, 306-313 (2000)); progression of autoimmune myocarditis (Yokoseki et al., Circ. Res. 89: 899-906 (2001)); acute rejection and graft arteriopathy in cardiac transplantation (Suzuki et al., Gene Therapy 7: 1847-1852 (2000)); and myocardial infarction (Morishita et al., Nature Medicine 3(8): 894-899 (1997)), NF-κB decoy molecules also find utility in the treatment of such diseases and conditions.

Recent evidence indicates that NF-κB and the signaling pathways that are involved in its activation are also important for tumor development. See, e.g. Karin et al., Nat. Rev. Cancer 2(4):301-10 (2002). Therefore, blocking NF-κB by the decoy molecules of the present invention finds utility in the prevention and treatment of cancer, offering a new anti-cancer strategy, either alone or in combination with other treatment options.

Many anti-inflammatory and anti-rheumatic drugs , including glucocorticoids, aspirin, sodium salicylate, and sulfosalazine, are inhibitors of NF-κB activation. For the treatment of inflammatory and autoimmune diseases and conditions, the NF-κB decoy molecules of the present invention can optionally be administered in combination with such drug treatments. Combination treatment includes simultaneous administration as well as consecutive administration of two or more drugs in any order.

E2F Oligonucleotide Decoy Molecules

Design of E2F Decoys with Improved Properties

It is well known that many transcription factors can bend the DNA upon binding to their recognition site and that nonlinear DNA structures facilitate and even determine proximal and distal DNA-protein contacts involved in transcription (Perez-Martin et al., Microbiol Rev 58:268-290 (1994); and van der Vliet and Verrijzer, Bioassays 15:25-32 (1993)). More recently, the E2F recognition site has been found to contain an intrinsic DNA bend (Cress and Nevins, Mol. Cel.. Biol. 16:2119-2127 (1996)). The binding of free E2F to this recognition site results in a DNA bend similar in magnitude to the intrinsic bend but in the opposite orientation. It is also known that the structure of the E2F-1 promoter affects the transcriptional activity of the promoter. Five base-pair substitutions in and around the E2F site change the DNA helix structure, E2F binding and influence transcriptional activity. The natural bend in the E2F binding sites together with the fact that E2F binding to the site has a dramatic effect on this structure seems to suggest a role for DNA structure in E2F binding and E2F-dependent transcriptional control. Binding of transcription factors to their binding sites is sensitive to the structure and shape of the DNA. The level of specificity of interaction is enhanced by flexibility and/or distortion in the DNA. For further details see, also Philos Trans R Soc Lond B Biol Sci 351:501-9 (1996) and Rhodes et al., Indian J. Biochem Biophys 33:83-7 (1996).

The present invention is based on the finding that by changing the shape and/or structure of an E2F decoy molecule, one can greatly improve its binding affinity to the target E2F transcription factor, which, in turn results in more effective inhibition of the biological function of the target E2F transcription factor.

In the Exanples provided herein, the shape/structure of the E2F decoy molecule has been changed by changing the sequences flaking the core binding sequence, which resulted in an order of a magnitude improvement in E2F binding affinity. The increased binding affinity makes the E2F decoy a much more potent inhibitor of E2F biological function. The shape and structure of the DNA are influenced by the base pair sequence, length of the DNA, backbone and nature of the nucleotide (i.e. native DNA vs. modified sugars or bases). Thus, the shape and/or structure of the molecule can also be changed by other approaches, such as, for example, by changing the total length, the length of the fully complementary, double-stranded region within the molecule, by alterations within the core and flanking sequences, by changing the backbone structure and by base modifications. E2F decoy molecules having increased binding affinity and/or improved in vivo stability can be designed and made by any of such approaches or by any combinations thereof.

In particular, by changing the core sequence (including its length, sequence, base modifications and backbone structure) it is possible to change the binding affinity, the stability and the specificity of the E2F decoy molecule. Changes in the flanking sequence have a genuine impact on and can significantly increase the in vivo stability of the molecule, and may affect binding affinity and/or specificity

Thus, in its broadest aspect, the invention concerns E2F decoy double-stranded oligodeoxynucleotide (dsODN) molecules, that have a flexible structure capable of changing shape and/or structure, e.g. bending and increased binding affinity to the target E2F transcription factor or factors. Thus, the E2F decoy molecules of the present invention can have increased binding affinity to one or more of E2F-1, E2F-2, E2F-3, E2F-4, E2F-5, and E2F-6.

In a more specific aspect, the present invention concerns E2F decoy double-stranded oligodeoxynucleotide (dsODN) molecules with improved properties. In particular, the invention concerns novel E2F decoy dsODN molecules, which have high binding affinity for an E2F transcription factor (including its heterodimer (E2F/DP) and homodimer (E2F/E2F) forms) and/or exhibit improved stability in vivo.

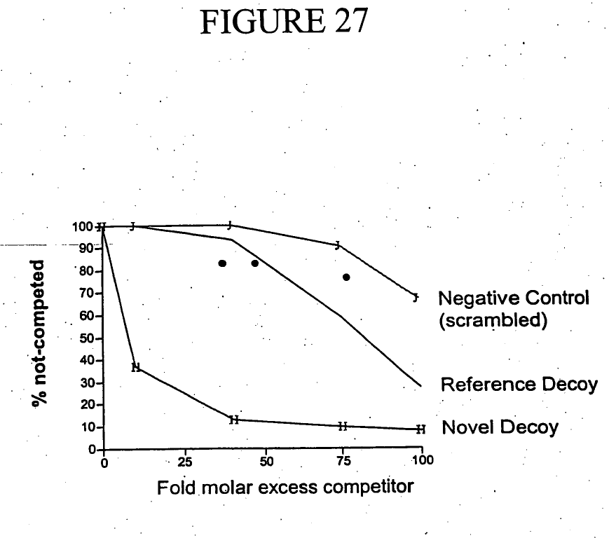

In one embodiment, the E2F decoy dsODN molecule comprises a core sequence that is capable of specific binding to an E2F transcription factor, flanked by 5′ and 3′ sequences, wherein (i) the core sequence consists of about 5 to 12, preferably about 6 to 10 base pairs; (ii) the molecule comprises an about 12 to 28, preferably about 14 to 24 base-pair long double-stranded region composed of two fully complementary strands; and (iii) the E2F decoy dsODN binds to the target E2F transcription factor with a binding affinity that is at least about 5-fold, more preferably at least about 7-fold, even more preferably at least about 10-fold, most preferably at least about 15-fold of the binding affinity of the reference decoy molecule of FIG. 26 (SEQ ID NOs: 94 and 95), as determined by a competitive gel mobility shift assay performed on nuclear extract from vascular smooth muscle cells (VSMCs), following the protocol described in Example 1. Preferably, the melting temperature (Tm) of the improved E2F decoy dsODN molecule is also significantly higher than the Tm of the reference decoy molecule of FIG. 26 (SEQ ID NOs: 94 and 95) (42.3° C.).

The length of the fully-complementary double-stranded portion of the E2F decoy molecule herein is believed to be important for enhanced binding affinity and stability. In order to achieve these improved properties, this region should contain at least about 12 base pairs, and typically its length is between about 12 and about 28 base pairs. The “fully complementary” region consists of two nucleotide strands where each nucleotide in the first strand undergoes Watson-Crick base pairing with each nucleotide in the second strand.

The core sequence typically should comprise at least 6 base pairs, and usually at least about 8 base pairs for satisfactory binding to the target E2F transcription factor. Generally, the core sequence consists of about 5 to 12, more typically about 6 to 10 base pairs. The core sequence may be or may contain sequences from the E2F binding sequences in the promoter region of a gene, the transcription of which is up- or down-regulated by an E2F transcription factor. Alternatively, the core sequence may be a synthetic sequence that does not occur in nature as an E2F binding sequence, such as a consensus sequence that is designed based on the nucleotide at each site which occurs most frequently in the E2F binding sequences of various genes, or binding sequences for various E2F transcription factors.

The flanking sequences are typically about 5 to 50 bases long, and can be, but need not be, fully complementary. Thus, the flanking region(s) may comprise single stranded overhangs at either end. It is believed that binding affinity and stability are affected more by the length and sequence of the truly double-stranded region, composed of two fully complementary strands within the oligonucleotide decoy molecules of the present invention than by the length of the flanking region(s) per se.

The nucleotide sequences present in the decoy molecules of the present invention may comprise modified or unusual nucleotides, and may have alternative backbone chemistries. Synthetic nucleotides may be modified in a variety of ways, see, e.g. Bielinska et al. Science 25);997 (1990). Thus, oxygens may be substituted with nitrogen, sulfur or carbon; phosphorys substituted with carbon; deoxyribose substituted with other sugars, or individual bases substituted with an unnatural base. In each case, any change will be evaluated as to the effect of the modification on the binding ability and affinity of the oligonucleotide decoy to the E2F trascription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g. Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β,D-mannosylqueosine, 5-methoxycarbonylmethyl-2-thiouridine, 5-metoxycarbonalmethyluridine, 5-methoxyuridine, 2-methylthio-N6-isopentenyladenosine, N-((9-beta-D-ribofuransyl-2-methylthiopurine-6-yl)carbamoyl)threonine, N-((9-beta-D-ribofuranosylpurine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuransylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl.

The E2F decoy molecules of the present invention can be synthesized by standard phosphodiester or phosphoramidate chemistry, using commercially available automatic synthesizers.

In a particular embodiment, the E2F decoy molecules of the present invention include a core sequence comprising a strand selected from the group consisting of

| | |
|---|---|
| TTTSGCGS | (SEQ ID NO: 100) |
| TTTGGCGC | (SEQ ID NO: 101) |
| TTTCGCGC | (SEQ ID NO: 102) |
| TTTCCCGC | (SEQ ID NO: 103) |
| TTTGCCGC | (SEQ ID NO: 104) |
| CTTCCCGC | (SEQ ID NO: 105) |
| GTTCCCGC | (SEQ ID NO: 106) |
| CTTCGCGC | (SEQ ID NO: 107) |
| TTAGCGCC | (SEQ ID NO: 108) |
| TGAGCGCC | (SEQ ID NO: 109) |
| GTAGCGCC | (SEQ ID NO: 110) |
| GGAGCGCC | (SEQ ID NO: 111) |
| CTAGCGCC | (SEQ ID NO: 112) |
| CGAGCGCC | (SEQ ID NO: 113) |
| GTTCGCGC | (SEQ ID NO: 114) |
| TTTGCGCC | (SEQ ID NO: 115) |
| TGTGCGCC | (SEQ ID NO: 116) |
| GTTGCGCC | (SEQ ID NO: 117) |
| GGTGCGCC | (SEQ ID NO: 118) |
| CTTGCGCC | (SEQ ID NO: 119) |
| CGTGCGCC | (SEQ ID NO: 120) |
| TTTCCGG | (SEQ ID NO: 121) |
| TTTCGCGG | (SEQ ID NO: 122) |
| GTTGGCGC | (SEQ ID NO: 123) |
| CTTGGCGC | (SEQ ID NO: 124) |
| CTTGCCGC | (SEQ ID NO: 125) |
| GTTGCCGC | (SEQ ID NO: 126) |
| TTTGGCGG | (SEQ ID NO: 127) |
| TTACCGCC | (SEQ ID NO: 128) |
| TGACCGCC | (SEQ ID NO: 129) |
| GTACCGCC | (SEQ ID NO: 130) |
| GGACCGCC | (SEQ ID NO: 131) |
| CTACCGCC | (SEQ ID NO: 132) |
| CGACCGCC | (SEQ ID NO: 133) |
| TTTCCGCC | (SEQ ID NO: 134) |
| TGTCCGCC | (SEQ ID NO: 135) |
| GTTCCGCC | (SEQ ID NO: 136) |
| GGTCCGCC | (SEQ ID NO: 137) |
| CTTCCGCC | (SEQ ID NO: 138) |
| CGTCCGCC | (SEQ ID NO: 139) |
| CTTCCCGG | (SEQ ID NO: 140) |
| TTTGCCGG | (SEQ ID NO: 141) |
| GTTCCCGG | (SEQ ID NO: 142) |
| CTTCGCGG | (SEQ ID NO: 143) |
| TTTGCGCG | (SEQ ID NO: 144) |
| TTAGCGCG | (SEQ ID NO: 145) |
| TGTGCGCG | (SEQ ID NO: 146) |
| TGAGCGCG | (SEQ ID NO: 147) |
| GTTGCGCG | (SEQ ID NO: 148) |
| GTAGCGCG | (SEQ ID NO: 149) |
| GGTGCGCG | (SEQ ID NO: 150) |
| TTTSGCGCGMNR | (SEQ ID NO: 151) |
| GTTGGCGG | (SEQ ID NO: 153) |
| GTTCGCGG | (SEQ ID NO: 154) |
| TTTCCCGG | (SEQ ID NO: 155) |
| CTTGCCGG | (SEQ ID NO: 156) |
| GTTGCCGG | (SEQ ID NO: 157) |
| TGTCGCGC | (SEQ ID NO: 158) |
| CTTCCCGG | (SEQ ID NO: 159) |
| CGTCGCGC | (SEQ ID NO: 160) |
| GGTCGCGC | (SEQ ID NO: 161) |
| TTTCGGGC | (SEQ ID NO: 162) |
| TGTGGCGC | (SEQ ID NO: 163) |
| TGTCGCGG | (SEQ ID NO: 164) |

and its complement where S is a G or a C:

Based on this information and other knowledge about the structure of intrinsic E2F binding sites, one skilled in the art can further optimize the structure of the E2F decoy molecules herein, for example, by known techniques of molecular modeling, co-crystallization with the E2F-DP complex, and other means known in the art. The actual sequence of the flaking regions near the core sequence is more critical than the sequence of more distant regions. Thus, the identity of the nucleotides at positions adjacent to or within a few nucleotides from the core sequence needs to be more carefully controlled than the identity of the nucleotides at positions farther away from the core sequence. In a particular embodiment, the flanking sequences are those shown in FIG. 26, SEQ ID NOS: 96 and 97, which can be coupled with any of the core sequences listed above.

As discussed earlier, E2F and DP proteins form heterodimers to give rise to E2F functional activity. In addition, homodimers of certain E2F proteins have also been described. Individual E2F-DP or E2F-E2F species invoke different transcriptional responses depending on the identity of the E2F moiety and the proteins that are associated with the complex. If desired, the E2F dsODN molecules can designed to exhibit preferential binding to one or more E2F transcription factors, which, in turn, is expected to result in different in vivo biological activities. Thus, E2F decoy molecules useful in cancer therapy can be designed by this approach.

The binding affinity of a candidate decoy molecule can be determined by standard methods, for example, by a gel shift mobility assay. The gel shift, or electrophoretic mobility shift (EMSA), assay provides a rapid and sensitive method for detecting the binding of transcription factors, or other DNA-binding proteins, to DNA. The assay is based on the observation that complexes of protein and DNA migrate through a non-denaturing polyacrylamide gel more slowly than free double-stranded oligonucleotides. The gel shift assay is performed by incubating a purified protein, or a complex mixture of proteins (such as nuclear extracts), with a ³²p end-labeled DNA fragment containing the transcription factor-binding site. The reaction products are then analyzed on a nondenaturing polyacrylamide gel. The specificity of the transcription factor for the binding site is established by competition experiments using excess amounts of oligonucleotides either containing a binding site for the protein of interest or a scrambled DNA sequence. The identity of proteins contained within a complex is established by using an antibody which recognizes the protein and then looking for either reduced mobility of the DNA-protein-antibody complex or disruption of the binding of this complex to the radiolabeled oligonucleotide probe.

Prime Therapeutic Targets

The E2F decoy molecules of the present invention are expected to find clinical use in the prevention and treatment of coronary heart disease, the single leading killer of American men and women, that caused over 450,000 deaths in the United States in 1998, according to the American Heart Association.

In addition, E2F decoys find utility in the treatment of peripheral vascular disease, which is characterized by atherosclerotic narrowing of peripheral arteries and, as a result, adversely affects blood circulation. In early clinical stages, the disease manifests itself in leg pain, but if left untreated, it can develop into gangrene, necessitating amputation of the limb, and substantial and irreversible morbidity and mortality.

A further clinical target for E2F decoys is neointimal hyperplasia, the pathological process that underlies graft atherosclerosis, stenosis, and the majority of vascular graft occlusion. Neointimal hyperplasia is commonly seen after various forms of vascular injury, and is a major component of the vein graft's response to harvest and surgical implantation into high-pressure arterial circulation.

In addition, an important role for E2F in the development of cancer has been suggested. As discussed earlier, E2F is responsible for inducing expression of a group of genes required for cell growth and cell division. When the cell receives growth inhibitory signals, E2F is inactivated by the tumor suppressor retinoblastoma gene, Rb. As a result, the growth control genes regulated by E2F remain inactive and the cell is held in a quiescent state. It has been proposed that in tumor cells which carry mutated copies of Rb, E2F is no longer controlled by Rb. As a result, E2F activates the genes directing cell division and so leaves the cell in a permanently proliferative state. For further details, including alternative mechanisms, see, e.g. Johnson and Schneider-Broussard, Front Biosci 3:d447-8 (1998). It has been reported that small peptides which inhibit E2F activity when introduced into tumor cell lines cause apoptosis (Bandara et al., Nature Biotechnology 15:896-901 (1997). Regardless of the underlying mechanism, the E2F decoy molecules of the present invention hold promise in the treatment of various types of cancer including breast cancer.

HIF-1 Oligonucleotide Decoy Molecules

In one embodiment of this invention, we systematically developed and optimized several sets of transcription factor decoys that specifically bind to transcription factor HIF-1.

1. Design of HIF-1 dsODN molecules

In one embodiment, the HIF-1 dsODN molecules of the present invention consist of two oligonucleotide strands which are attached to each other by Watson-Crick base pairing. While typically all nucleotides in the two strands participate in the base pairing, this is not a requirement. Oligonucleotide decoy molecules, where one or more, such as 1-3 or 1 or 2 nucleotides are not involved in base pairing are also included. In addition, the double stranded decoys may contain 3′ and/or 5′ single stranded overhangs.

In another embodiment, the HIF-1 dsODN molecules of the present invention comprise two oligonucleotide strands which are attached to each other by Watson-Crick base pairing, and are additionally covalently attached to each other at either the 3′ or the 5′ end, or both, resulting in a dumbell structure, or a circular molecule. The covalent linkage may be provided, for example, by phosphodiester linkages or other linking groups, such as, for example, phosphothioate, phosphodithioate, or phosphoamidate linkages.

Generally, the dsODN molecules of the invention comprise a core sequence that is capable of specific binding to a HIF-1 transcription factor, flanked by 5′ and/or 3′ sequences, wherein the core sequence consists of about 5 to 14, or about 6 to 12. or about 7 to about 10 base pairs; and the flanking sequences are about 2 to 8, or about 2 to 6, or about 2 to 4 base pairs long. The molecule typically comprises an about 12 to 28, preferably about 14 to 24 base-pair long double-stranded region composed of two fully or partially complementary strands (including the core and flaking sequences).

Changing the core sequence (including its length, sequence, base modifications and backbone structure) it is possible to change the binding affinity of the HIF-1 decoy molecule. In addition, changes in the flanking sequence have a genuine impact on and can significantly increase the in vivo stability of the HIF-1 decoy molecule, and may affect binding affinity and/or specificity. In particular, the shape/structure of the HIF-1 decoy molecule can be changed by changing the sequences flaking the core binding sequence, which can result in improved stability and/or binding affinity. The shape and structure of the DNA are influenced by the base pair sequence, length of the DNA, backbone and nature of the nucleotide (i.e. native DNA vs. modified sugars or bases). Thus, the shape and/or structure of the molecule can also be changed by other approaches, such as, for example, by changing the total length, the length of the fully complementary, double-stranded region within the molecule, by alterations within the core and flanking sequences, by changing the backbone structure and by base modifications.

The nucleotide sequences present in the decoy molecules of the present invention may comprise modified or unusual nucleotides, and may have alternative backbone chemistries. Synthetic nucleotides may be modified in a variety of ways, see, e.g. Bielinska et al. Science 250:997-1000 (1990). Thus, oxygens may be substituted with nitrogen, sulfur or carbon; phosphorus substituted with carbon; deoxyribose substituted with other sugars, or individual bases substituted with an unnatural base. Thus replacement of non-bridging oxygen atoms of the internucleotide linkage with a sulfur group (to yield a phosphorothioate linkage) has been useful in increasing the nuclease resistance of the dsODN molecule. Experiments determining the relationship between the number of sulfur modifications and stability and specificity of the HIF-1 dsODN molecules herein are set forth in the Example below.

In each case, any change will be evaluated as to the effect of the modification on the binding ability and affinity of the oligonucleotide decoy to the HIF-1 transcription factor, effect on melting temperature and in vivo stability, as well as any deleterious physiological effects. Such modifications are well known in the art and have found wide application for anti-sense oligonucleotide, therefore, their safety and retention of binding affinity are well established (see, e.g. Wagner et al. Science 260:1510-1513 (1993)).

Examples of modified nucleotides, without limitation, are: 4-acetylcytidin, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β,D-galactosylqueuosine, 2′-O-methylguanosine, inosine, N6-isopentenyladenosine 1-metyladenosine, 1-methylpseudouridine, 1-methylguanosine, 1-methylinosine, 2,2-dimethylguanosine, 2-methyladenosine, 2-methylguanosine 3-methylcytidine 5-methylcytidine, N6-methyladenosine, 7-methylguanosine, 5-methylaminomethyl-2-thiouridine, β,D-mannosylqueosine, 5-methoxycarbonylmethyl-2-thiouridine, 5-metoxycarbonalmethyluridine, 5-methoxyuridine, 2-methylthio-N6-isopentenyladenosine, N-((9-beta-D-ribofuransyl-2-methylthiopurine-6-yl)carbamoyl)threonine, N-((9-beta-D-ribofuranosyl)purine-6-yl)N-methylcarbamoyl)threonine, uridine-5-oxyacetic acid-methylester uridine-5-oxyacetic acid, wybutoxosine, pseudouridine queuosine, 2-thiocytidine, 5-methyl-2-thiouridine, 2-thiouridine, 4-thiouridine, 5-methyluridine, N-((9-beta-D-ribofuranosylpurine-6-yl)-carbamoylthreonine, 2′-O-methyl-5-methyluridine, 2′-O-methyluridine, 3-(3-3-amino-3-carboxy-propyl)uridine(acp3)u, and wybutosine.

In addition, the nucleotides can be linked to each other, for example, by a phosphoramidate linkage. This linkage is an analog of the natural phosphodiester linkage such that a bridging oxygen (—O—) is replaced with an amino group (—NR—), wherein R typically is hydrogen or a lower alkyl group, such as, for example, methyl or ethyl. Other likages, such as phosphothioate, phosphodithioate, etc. are also possible.

The decoys of the present invention can also contain modified or analogous forms of the ribose or deoxyribose sugars generally present in polynucleotide structures. Such modifications include, without limitation, 2′-substituted sugars, such as 2′-O-methyl-, 2′-O-allyl, 2′-fluoro- and 2′azido-ribose, carboxylic sugar analogs, α-anomeric sugars, epimeric sugars, such as arabinose, xyloses, lyxoses, pyranose sugars, furanose sugars, sedoheptuloses, acyclic analogs and abasic nucleoside analogs, such as methyl riboside.

In general, the oligonucleotide decoys of the present invention are preferably comprised of greater than about 50%, more preferably greater than about 80%, most preferably greater than about 90% conventional deoxyribose nucleotides.

The HIF-1 dsODN decoys of the present invention can be further modified to facilitate their localization, purification, or improve certain properties thereof. For example, a nuclear localization signal (NLS) can be attached to the decoy molecules, in order to improve their delivery to the cell nucleus.

In addition, the HIF-1 decoy molecules of the invention may be conjugated with carrier molecules, such as peptides, proteins or other types of molecules, as described, for example, in the following references: Avrameas et al., J. Autoimmun 16, 383-391 (2001); Avrameas et al., Bioconjug. Chem. 10: 87-93 (1999); Gallazzi et al., Bioconjug. Chem. 14, 1083-1095 (2003); Ritter, W. et al., J. Mol. Med. 81, 708-717 (2003).

The HIF-1 decoy molecules of the invention may further be derivatized to include delivery vehicles which improve delivery, distribution, target specific cell types or facilitate transit through cellular barriers. Such delivery vehicles include, without limitation, cell penetration enhancers, liposomes, lipofectin, dendrimers, DNA intercalators, and nanoparticles.

2. Synthesis of HIF-1 dsODN Molecules

The HIF-1 dsODN decoy molecules of the present invention can be synthesized by standard phosphodiester or phosphoramidate chemistry, using commercially available automatic synthesizers. The specific dsODN molecules described in the example have been synthesized using an automated DNA synthesizer (Model 380B; Applied Biosystems, Inc., Foster City, Calif.). The decoys were purified by column chromatography, lyophilized, and dissolved in culture medium. Concentrations of each decoy were determined spectrophotometrically.

3. Characterization of HIF-1 dsODN Molecules

The HIF-1 decoy molecules of the present invention can be conveniently tested and characterized in a gel shift, or electrophoretic mobility shift (EMSA) assay. This assay provides a rapid and sensitive method for detecting the binding of transcription factors to DNA. The assay is based on the observation that complexes of protein and DNA migrate through a non-denaturing polyacryamide gel more slowly than free double-stranded oligonucleotides. The gel shift assay is performed by incubating a purified protein, or a complex mixture of proteins (such as nuclear extracts), with a ³²p end-labeled DNA fragment containing a transcription factor-binding site. The reaction products are then analyzed on a non-denaturing polyacrylamide gel. The specificity of the transcription factor for the binding site is established by competition experiments, using excess amounts of oligonucleotides either containing a binding site for the protein of interest or a scrambled DNA sequence. The identity of proteins contained within a complex is established by using an antibody which recognizes the protein and then looking for either reduced mobility of the DNA-protein-antibody complex or disruption of the binding of this complex to the radiolabeled oligonucleotide probe.

4. Use of HIF-1 dsODN Molecules

As discussed before HIF-1 has been shown to play a critical role in tumor growth, including angiogenesis and glycolysis, and metastases, and identified as a potential target for anti-cancer therapeutic strategies. (Semenza, Nature Rev. 3:721-732 (2003); Williams et al., Oncogene 21:282-90 (2002); Griffiths et al., Cancer Res. 62:688-95 (2002); Welsh et al., Mol. Cancer Ther. 2:235-43 (2003)). HIF-1 has been shown to be overexpressed in breast cancer and potentially associated with more aggressive tumors (Bos et al., J. Natl. Cancer Inst. 93:309-314 (2001)). In addition, HIF-1 has been recently identified as a critical link between inflammation and oncogenesis (Jung et al., The FASEB Joumak Express Article 10.1096/fj.03-0329fjc, published online September 4, 2003). HIF-1α overexpression in biopsies of brain, breast, cervical, esophageal, oropharyngeal and ovarian cancers is correlated with treatment failure and mortality. Increased HIF-1 activity promotes tumor progression, and inhibition of HIF-1 could represent a novel approach to cancer therapy. Therefore, blocking HIF-i by the decoy molecules of the present invention finds utility in the prevention and treatment of cancer, offering a new anti-cancer strategy, either alone or in combination with other treatment options. Inhibition of HIF-1 by administering the dsODN molecules of the present invention may also enhance the efficacy of other cancer therapies, such as radiation therapy and/or treatment with chemotherapeutic agents. Specific cancer targets include, without limitation, solid tumor malignancies and Non-Hodgkin's lymphoma.

In addition, HIF-1 has been identified as a target for diseases in general in which hypoxia is a major aspect, such as, for example, heart disease and stroke (Giaccia et al., Nat. Rav. Drug Discov. 2:803-822 (2003)). Accordingly, the HIF-1 decoy molecules of the present invention can also be used for the prevention and treatment of hypoxia-associated pathological diseases and conditions, such as, for example, cardiovascular diseases, such as myocardial ischemia, myocardial infarction, congestive heart failure, cardiomyopathy, cardiac hypertrophy, and stroke.

HIF-1 decoy molecules additional find utility in ophthalmology, including diabetic retinopathy, which is the leading cause of blindness in the United States. Additional opthalmologic targets include Age-related Macular Degeneration (AMD), and corneal neovascularization associated with transplants.

HIF-1 dsODN molecules find additional use in the prevention and treatment of pathogenic blood vessel growth, associated, for example, with psoriasis, corneal neovascularization, infection or trauma.

Increased angiogenesis is also a key component of synovitis and bone modeling in arthritis. Preclinical studies of angiogenesis inhibitors in animals models of inflammatory arthritis support the hypothesis that inhibition of neovascularization may reduce inflammation and joint damage. Therefore, additional therapeutic targets include inflammatory diseases, including arthritis, such as rheumatoid arthritis (RA), and musculoskeletal disorders. For further details see, e.g. Walsh and Haywood, Curr Opin Investig Drugs. 2(8):1054-63 (2001). In addition, similar to tumor growth, endometriotic implants require neovascularization to establish, grow and invade. This process can be blocked by the HIF-1 decoys of the present invention. See also, Taylor et al.. Ann N Y Acad Sci. 955:89-100 (2002).

Administration of Decoy Molecules

A preferred mode of delivering the decoy molecules of the present invention is through the use of formulations decribed above and as shown in the Examples below.

When administered in liposomes, the decoy concentration in the lumen will generally be in the range of about 0.1 μM to about 50 μM per decoy, more usually about 1 μM to about 10 μM, most usually about 3 μM.

The determination of the appropriate concentrations and doses is well within the competence of one skilled in the art. Optimal treatment parameters will vary depending on the indication, decoy, clinical status of the patient, etc., and can be determined empirically based on the instructions provided herein and general knowledge in the art.

The decoys may be administered as compositions comprising individual decoys or mixtures of decoys. Usually, a mixture contains up to 6, more usually up to 4, more usually up to 2 decoy molecules.

In cancer therapy, the administration of decoy molecules can be combined with other treatment options, including treatment with chemotherapeutic anticancer agents and/or radiation therapy.

Ultrasound-Mediated Delivery of Decoy Molecules

In one aspect of the invention, application of ultrasound has been demonstrated to enhance transdermal drug transport, a phenomenon referred to as sonophoresis. We intend to evaluate this method for topical delivery of our transcription factor decoy therapeutics in several target clinical indications relating to skin inflammation (atopic dermatitis and psoriasis) and joint inflammation (rheumatoid and osteo arthritis). Proper choice of ultrasound parameters including frequency, intensity, pulse length, and transducer distance from the skin is critical for efficient sonophoresis. Based on the successful uses of this method as reported in the literature, we will begin by assessing two sonophoresis treatment strategies: 1) therapeutic frequency sonophoresis (1 MHz frequency, 2 W/cm intensity); 2) low-frequency sonophoresis (20 kHz frequency, up to 225 mW/cm² intensity). Therapeutic frequency sonophoresis (1 MHz) is the most commonly used ultrasound frequency range for sonophoresis and has demonstrated typical enhancements of 10-fold or less for a number of low molecular weight drugs, which may limit its uses to local as opposed to systemic delivery. Low-frequency sonophoresis (20 kHz) has been shown to enhance transdermal transport of various low-molecular weight drugs as well as high-molecular weight proteins up to 48 kDA and may enhance transdermal permeability up to 1000-fold higher than therapeutic ultrasound. It is our hope that the use of sonophoresis in conjunction with and without our proprietary penetration enhancing formulations will enable more efficient drug transfer and allow more improved delivery to various target organs;

Further details of the invention will be apparent from the following examples which illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### EXAMPLES

Various methods for producing formulations are well known in the art. Therefore, preparing formulations having the components as described below is believed to be within the skill of those in the art.

Formulation compositions are exemplified in the tables below. Percentages are by weight, unless otherwise stated.

The gel-based formulations were prepared by combining the appropriate amounts of Parts A and B into two separate glass containers. After parts A and B were completely dissolved, they were mixed together under vigorous stirring using a three blade impeller. The emulsion and liposome based formulations were prepared by combining the appropriate amounts of Parts A and B into two separate glass containers. Parts A and B were heated to 65° C. until completely dissolved. Both parts were mixed together under vigorous stirring using a three blade impeller. The mixture was allowed to cool to room temperature while stirring.

### Example 1

### Aqueous Gel-Based Formulations

An aqueous gel-based formulation having the following ingredients was prepared:

Formulation F1

| Part | Ingredient | Weight percent |
|---|---|---|
| A | HPMC 4000 cps | 1.5 |
| | 1x Phosphate Buffered Saline | 48.7 |
| B | Ethanol | 49.0 |
| | Sodium laureth sulfate | 0.8 |

An aqueous gel-based formulation having the following ingredients was prepared:

Formulation F2

| Part | Ingredient | Weight percent |
|---|---|---|
| A | HPMC 4000 cps | 2.0 |
| | 100 mM Phosphate buffer | 10.0 |
| | 1.5 M NaCl | 10.0 |
| | Water | 67.0 |
| B | Ethanol | 10.0 |
| | Sodium laureth sulfate | 0.8 |
| | Methyl paraben | 0.1 |
| | Propyl paraben | 0.1 |

An aqueous gel-based formulation having the following ingredients was prepared:

Formulation F3

| Part | Ingredient | Weight percent |
|---|---|---|
| A | HPMC 4000 cps | 2.0 |
| | 100 mM Phosphate buffer | 10.0 |
| | 1.5 M NaCl | 10.0 |
| | Water | 72.0 |
| B | Ethanol | 5.0 |
| | Sodium laureth sulfate | 0.8 |
| | Methyl paraben | 0.1 |
| | Propyl paraben | 0.1 |

An aqueous gel-based formulation having the following ingredients was prepared:

Formulation F4

| Part | Ingredient | Weight percent |
|---|---|---|
| A | HPMC 4000 cps | 2.0 |
| | 100 mM Phosphate buffer | 10.0 |
| | 1.5 M NaCl | 10.0 |
| | Water | 76.0 |
| B | Ethanol | 1.0 |
| | Sodium laureth sulfate | 0.8 |
| | Methyl paraben | 0.1 |
| | Propyl paraben | 0.1 |

An aqueous gel-based formulation having the following ingredients was prepared:

Formulation F5

| Part | Ingredient | Weight percent |
|---|---|---|
| A | HPMC 4000 cps | 2.0 |
| | 100 mM Phosphate buffer | 10.0 |
| | 100 mM MgCl₂ | 10.0 |
| | Water | 28.0 |
| B | Ethanol | 49.0 |
| | Sodium laureth sulfate | 0.8 |
| | Methyl paraben | 0.1 |
| | Propyl paraben | 0.1 |

An aqueous gel-based formulation having the following ingredients was prepared:

Formulation F6

| Part | Ingredient | Weight percent |
|---|---|---|
| A | HPMC 4000 cps | 2.0 |
| | 100 mM Phosphate buffer | 10.0 |
| | 1.5 M NaCl | 10.0 |
| | Water | 57.0 |
| B | Ethanol | 20.0 |
| | Sodium laureth sulfate | 0.8 |
| | Methyl paraben | 0.1 |
| | Propyl paraben | 0.1 |

An aqueous gel-based formulation having the following ingredients was prepared:

Formulation F7

| Part | Ingredient | Weight percent |
|---|---|---|
| A | HPMC 4000 cps | 2.0 |
| | 100 mM Phosphate buffer | 10.0 |
| | 1.5 M NaCl | 10.0 |
| | Water | 28.0 |
| B | Ethanol | 49.0 |
| | Sodium laureth sulfate | 0.8 |
| | Methyl paraben | 0.1 |
| | Propyl paraben | 0.1 |

### Example 2

### Preparation of Liposome-Containing Formulations

A liposome-containing formulation having the following ingredients was prepared:

Formulation F8

| | | Weight |
|---|---|---|
| Part | Ingredient | percent |
| A | Hydroxyethylcellose (NATROSOL ®) | 1.0 |
| | Water | 76.8 |
| B | Phosphatidylcholine (phosphoLipon 90-H) | 10.0 |
| | Propylene glycol | 5.0 |
| | Ethanol | 5.0 |
| | Vitamin E-acetate | 1.0 |
| | N-Lauroylsarcosine | 0.6 |
| | Sorbitan monolaurate 20 (Span 20) | 0.4 |
| | Methyl paraben | 0.1 |
| | Propyl paraben | 0.1 |

A liposome-containing formulation having the following ingredients was prepared:

Formulation F9

| Part | Ingredient | Weight percent |
|---|---|---|
| A | Hydroxyethylcellose (NATROSOL ®) | 1.0 |
| | Water | 72.0 |
| B | Phosphatidylcholine (phosphoLipon 90-H) | 10.0 |
| | Propylene glycol | 5.0 |
| | Ethanol | 10.0 |
| | Vitamin E-acetate | 1.0 |
| | Sodium laureth sulfate | 0.8 |
| | Methyl paraben | 0.1 |
| | Propyl paraben | 0.1 |

A liposome-containing formulation having the following ingredients was prepared:

Formulation F10

| Part | Ingredient | Weight percent |
|---|---|---|
| A | Water | 52.0 |
| | 100 mM Phosphate buffer | 10.0 |
| | 1.5 M NaCl | 10.0 |
| | Hydroxyethylcellose (NATROSOL ®) | 1.0 |
| B | Phosphatidylcholine (phosphoLipon 90-H) | 10.0 |
| | Propylene glycol | 5.0 |
| | Ethanol | 10.0 |
| | Vitamin E-acetate | 1.0 |
| | Sodium laureth sulfate | 0.8 |
| | Methyl paraben | 0.1 |
| | Propyl paraben | 0.1 |

A liposome-containing formulation having the following ingredients was prepared:

Formulation F11

| Part | Ingredient | Weight percent |
|---|---|---|
| A | Water | 57.0 |
| | 100 mM Phosphate buffer | 10.0 |
| | 1.5 M NaCl | 10.0 |
| | Hydroxyethylcellose (NATROSOL ®) | 1.0 |
| B | Phosphatidylcholine (phosphoLipon 90-H) | 10.0 |
| | Propylene glycol | 5.0 |
| | Ethanol | 5.0 |
| | Vitamin E-acetate | 1.0 |
| | Sodium laureth sulfate | 0.8 |
| | Methyl paraben | 0.1 |
| | Propyl paraben | 0.1 |

A liposome-containing formulation having the following ingredients was prepared:

Formulation F12

| Part | Ingredient | Weight percent |
|---|---|---|
| A | Water | 59.5 |
| | 100 mM Phosphate buffer | 10.0 |
| | 1.5 M NaCl | 10.0 |
| | Hydroxyethylcellose (NATROSOL ®) | 1.0 |
| B | Phosphatidylcholine (phosphoLipon 90-H) | 10.0 |
| | Propylene glycol | 5.0 |
| | Ethanol | 2.5 |
| | Vitamin E-acetate | 1.0 |
| | Sodium laureth sulfate | 0.8 |
| | Methyl paraben | 0.1 |
| | Propyl paraben | 0.1 |

### Example 3

### Emulsion-Based Formulations

An emulsion-based formulation having the following ingredients was prepared:

Formulation F13

| Part | Ingredient | Weight percent |
|---|---|---|
| A | HPMC 4000 cps | 0.5 |
| | Water | 62.5 |
| B | Polyoxyl-40 stearate | 15.0 |
| | Glyceryl monostearate | 10.0 |
| | Isopropyl myristate | 10.0 |
| | N-Lauroylsarcosine | 0.6 |
| | Sorbitan monolaurate 20 (Span 20) | 0.4 |
| | Methyl paraben | 0.5 |
| | Propyl paraben | 0.5 |

An emulsion-based formulation having the following ingredients was prepared:

Formulation F14

| Part | Ingredient | Weight percent |
|---|---|---|
| A | HPMC 4000 cps | 0.5 |
| | Water | 63.0 |
| B | Polyoxyl-40 stearate | 15.0 |
| | Glyceryl monostearate | 10.0 |
| | Isopropyl myristate | 10.0 |
| | Sodium laureth sulfate | 0.35 |
| | 1-phenyl piperazine | 0.15 |
| | Methyl paraben | 0.5 |
| | Propyl paraben | 0.5 |

An emulsion-based formulation having the following ingredients was prepared:

Formulation F15

| Part | Ingredient | Weight percent |
|---|---|---|
| A | HPMC 4000 cps | 0.5 |
| | Water | 62.7 |
| B | Polyoxyl-40 stearate | 15.0 |
| | Glyceryl monostearate | 10.0 |
| | Isopropyl myristate | 10.0 |
| | Sodium laureth sulfate | 0.8 |
| | Methyl paraben | 0.5 |
| | Propyl paraben | 0.5 |

### Example 4

### Delivery of an Aqueous Gel-Based Formulation Containing NF-κB Decoy Molecule—Murine Model

This example shows that NF-κB decoy molecules, when administered using an aqueous gel-based formulation of the present invention, effectively permeates the skin and reduces the skin inflammation.

The basic protocol for this assay is described in Sasakawa et al., Int. Arch. Allergy Immunol., 126(3): 239-247 (2001) and Matsuoka et al., Allergy, 58(2): 139-145 (2003).

Specific pathogen-free (SPF) NC/Nga female mice (4-5 weeks) were purchased from Charles Rivers Japan (Yokohama, Japan). The animals were maintained under SPF conditions until 6 weeks of age. The mice were then injected intradermally with 5 mg/20 μl of Dermatophagoides pteronyssinus (Dp) extract (Greer Laboratories, Lenoir, N.C.) dissolved in saline, on the ventral side of their right ear on days 0, 2, 4, 7, 9 and 10. Twenty microliters of a vehicle containing ±0.1, 0.25, 0.5 or 1% NF-κB decoy was applied 2 times/day on the dorsal side of the Dp-treated ears 1-2 days after the last Dp injection for a total of 14 days. Degree of inflammation was measured indirectly by measuring the skin thickness of the ear. Skin thickness correlates very well with the amount of inflammatory cells in the dermis and epidermis as shown by histology. Ear thickness was measured 24 hours after each Dp injection and then every other day during the treatment regimen with a modified spring micrometer (Oditest, Dyer, Co., Lancaster, Calif.).

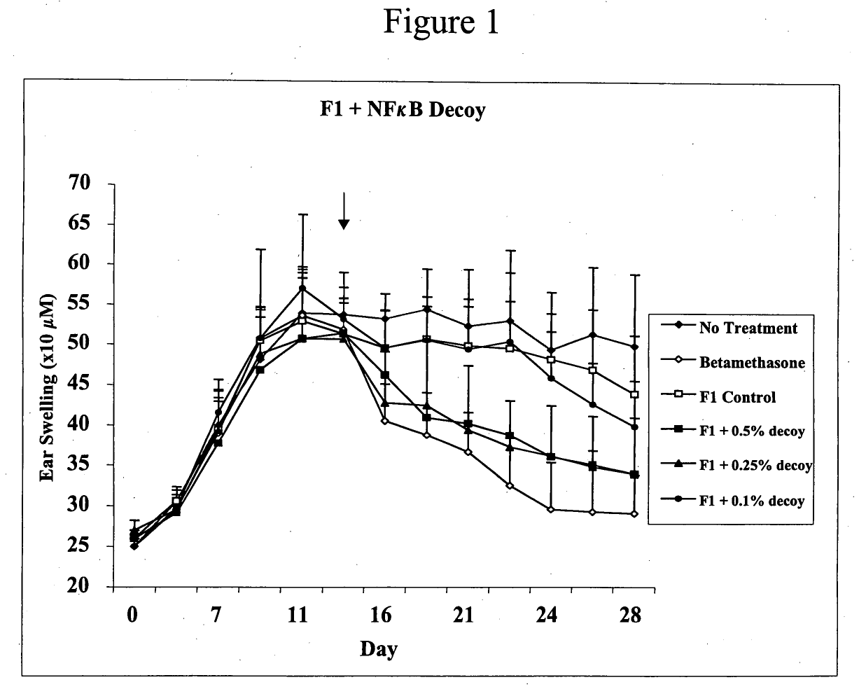

FIG. 1 shows dose titration of NF-κB decoy molecules in aqueous gel-base formulation F1 containing 0.8% sodium laureth sulfate, 49% ethanol, 1.5% HPMC 4000 cps and 48.7% 100 mM phosphate buffer. FIG. 1 shows that a treatment with 0.1, 0.25 and 0.5% of the NF-κB decoy molecule reduces ear swelling in a dustmite antigen induced atopic dermatitis. The graph illustrates the effectiveness of the NF-κB decoy molecule treatment when compared to the mouse skin without any treatment, treatment with formulation containing no NF-κB decoy molecule and treatment with betamethasone.

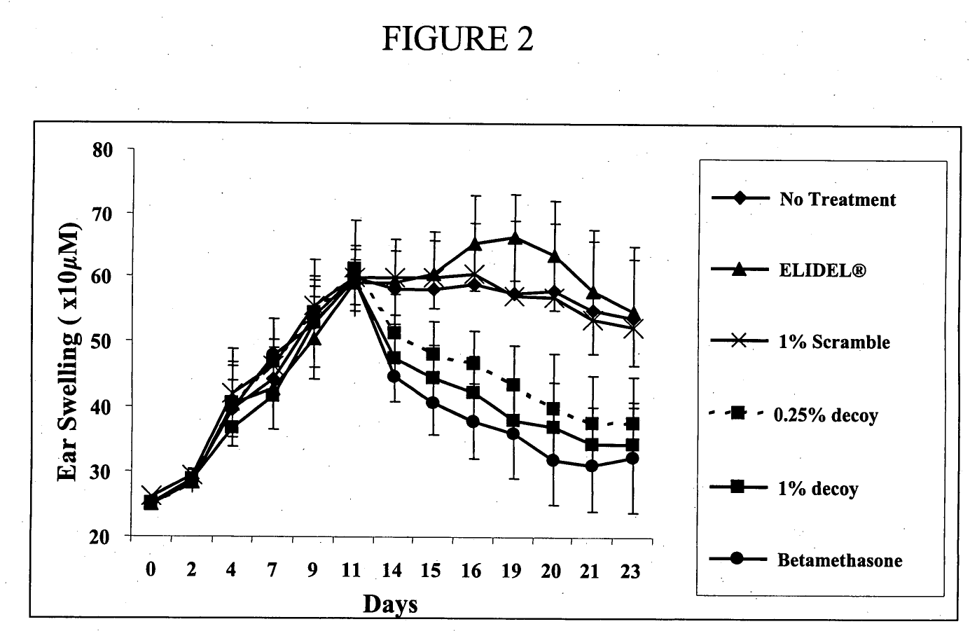

FIG. 2 further shows dose titration of NF-κB decoy molecules in aqueous gel-base formulation F6 containing 0.8% sodium laureth sulfate, 20% ethanol, 2.0% HPMC 4000 cps and 10.0% 100 mM phosphate buffer. FIG. 2 shows that a treatment with 0.25 and 1% of the NF-κB decoy molecule reduces ear swelling in dustmite Ag (Dp) induced contact dermatitis in NC/Nga mice. The graph illustrates the effectiveness of the NF-κB decoy molecule treatment when compared to the mouse skin without any treatment, treatment with ELIDEL® (Novartis AG Corp., Basel, Switzerland), treatment with formulation containing 1% scramble decoy molecule and treatment with betamethasone.

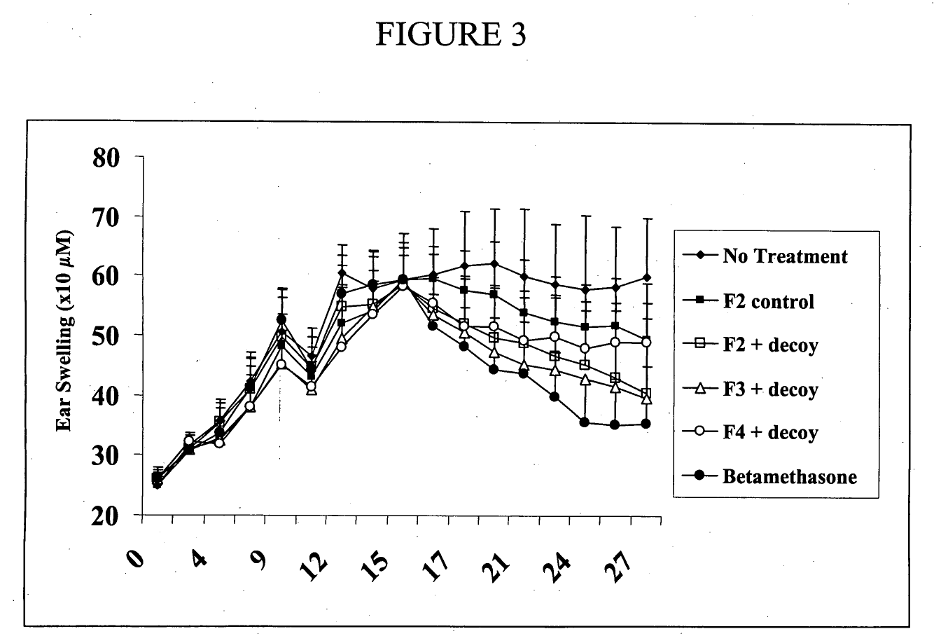

FIG. 3 is a graph showing the effectiveness of aqueous gel-based formulations containing various ethanol concentrations and 0.25% NF-κB decoy molecules. Formulation designated as F2 contains 10% ethanol, F3 contains 5% ethanol and F4 contains 1% ethanol. The formulation designated as F2 control contains no NF-κB decoy molecules. The results were compared to the samples receiving no treatment and those treated with betamethasone.

### Example 5

### Anti-Inflammatory Effects of NF-κB Decoy Molecule—Murine Model

This example shows that NF-κB decoy molecules, when administered using an aqueous gel-based formulation of the present invention, effectively decrease the gene expression of the pro-inflammatory cytokines, such as IL-1-1β, IL-6, TNFα and TSLP (thymic stromal lymphopoietin) in dustmite Ag (Dp) induced contact dermatitis in NC/Nga mice.

Dustmite antigen induced atopic dermatitis was induced as previously described above (Sasakawa, et al., 2001). Right ears of Dp-injected mice were removed 1 day after the final decoy treatment. Part of the ear was flash frozen in liquid nitrogen and store at −80 ° C. Total RNA was isolated from the ears using QIAZOL® lysis reagent (Qiagen, Amtsgericht Dusseldorf, Germany) according to manufacturer's instruction. The expression of the mouse genes was assayed by real-time quantitative PCR with an ABI PRISM® 7900 Sequence Detection System (Applied Biosystems, Foster City, Calif.). All procedures were carried out as previously described (Hurst et al., J. Immunol., 169(1):443-453 (Jul. 1, 2002)). Dnase-treated total RNAs were briefly mixed with random hexamers (Gibco-BRL, Carlsbad, Calif.), Oligo dt (Boehringer, Germany), and the first strand cDNAs were synthesized with SurperScript II™ reverse transcriptase (Gibco-BRL, Carlsbad, Calif.). Primers for the respective genes were designed using the primer design software PRIMER EXPRESS® (Applied Biosystems, Foster City, Calif.). Primers were synthesized at Sigma Genosys (Woodlands, Tex.). The quantitative PCR was performed using TAQMAN® PCR reagent kit (Applied Biosystems, Foster City, Calif.) according to the manufacturer's protocol. Sample cDNAs equivalent to 25 ng of RNA were examined in each reaction in a 384-well PCR plate. Levels of ubiquitin were measured for each sample, and used as internal standard. Cytokine levels are expressed as relative expression to ubiquitin levels.

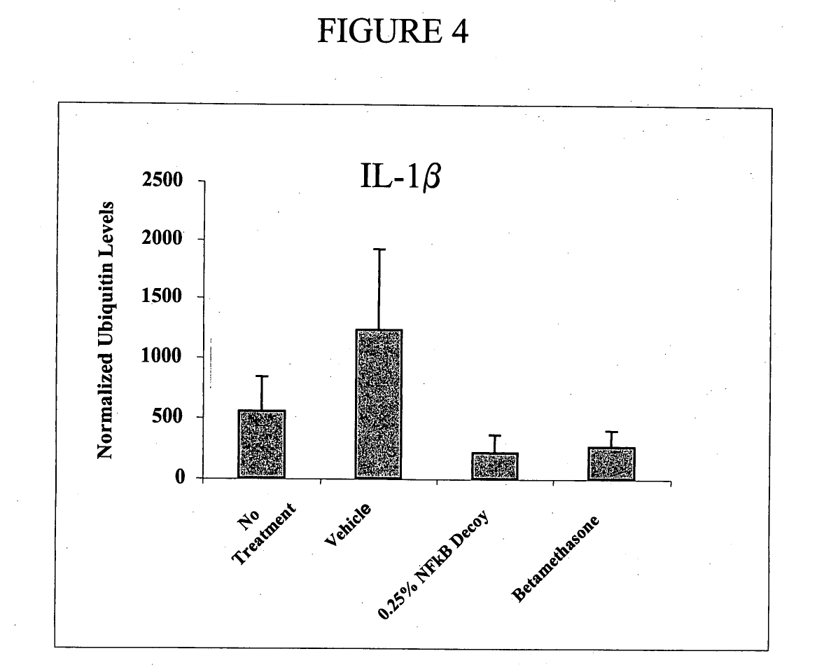

FIG. 4 is a graph showing the reduction of relative IL-1β gene expression levels in dustmite Ag (Dp) induced contact dermatitis in NC/Nga mice when treated with aqueous gel-based formulation F2 containing 0.25% NF-κB decoy molecules.

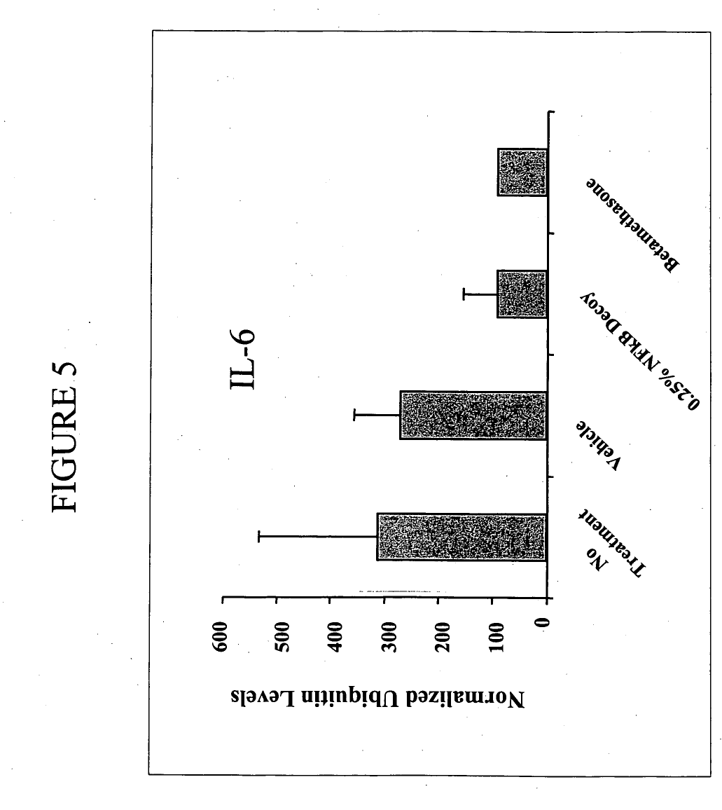

FIG. 5 is a graph showing the reduction of relative IL-6 gene expression levels in dustmite Ag (Dp) induced contact dermatitis in NC/Nga mice when treated with aqueous gel-based formulation F2 containing 0.25% NF-κB decoy molecules.

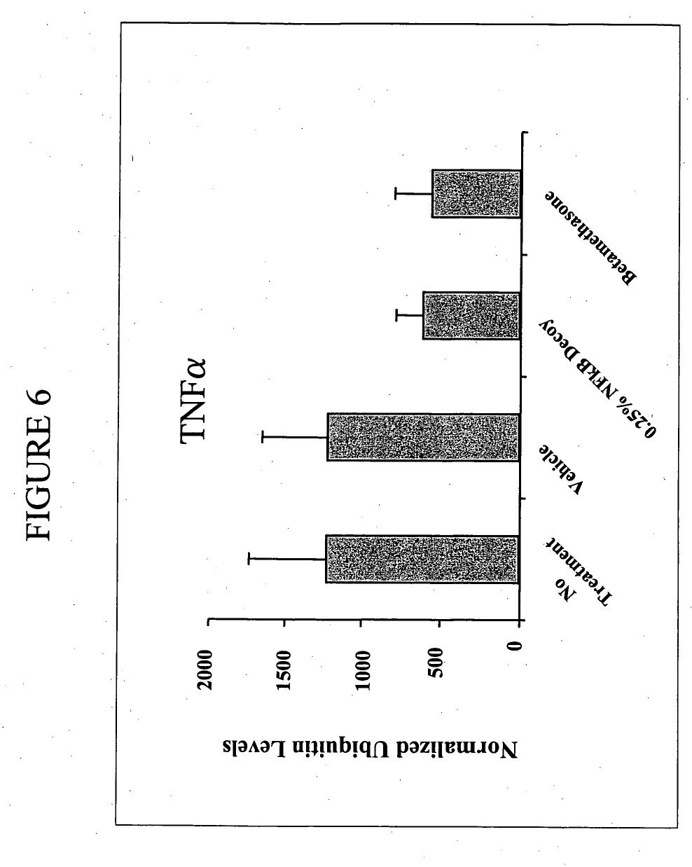

FIG. 6 is a graph showing the reduction of relative TNFA gene expression levels in in dustmite Ag (Dp) induced contact dermatitis in NC/Nga mice when treated with aqueous gel-based formulation F2 containing 0.25% NF-κB decoy molecules.

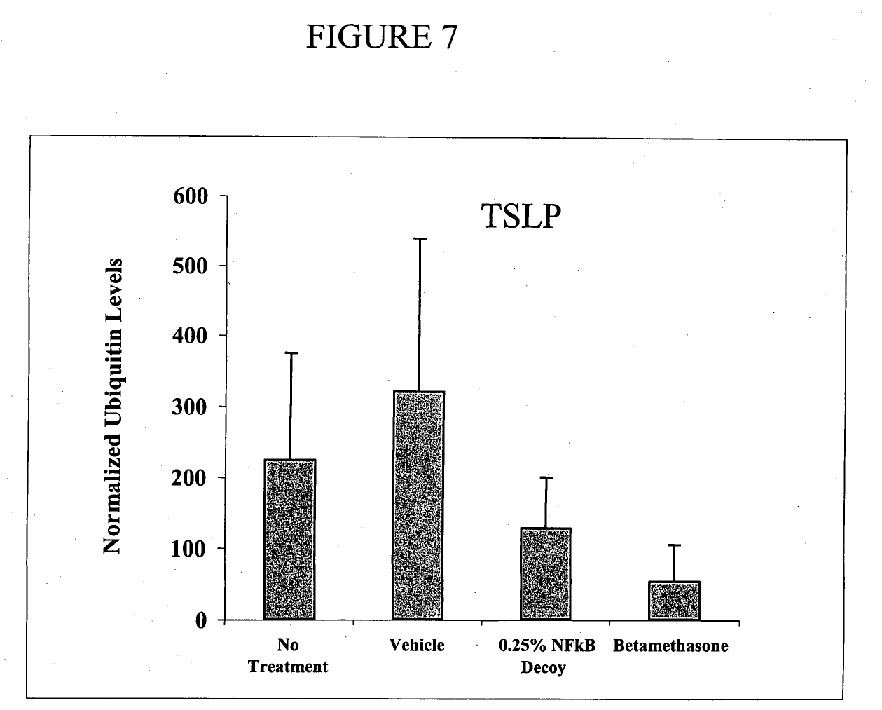

FIG. 7 is a graph showing the reduction of relative TSLP gene expression levels in in dustmite Ag (Dp) induced contact dermatitis in NC/Nga mice when treated with aqueous gel-based formulation F2 containing 0.25% NF-κB decoy molecules.

### Example 6

### Delivery of an Aqueous-Gel Based Formulation Containing NF-κB Decoy Molecule—Murine Model

This example further illustrates the efficacy of NF-κB decoy molecule in dustmite Ag (Dp) induced contact dermatitis in NC/Nga mice. The aqueous gel-based formulation, Fl, used in this experiment contained 0.8% sodium laureth sulfate, 49% ethanol, 1.5% HPMC 4000 cps and 48.7% 100 mM phosphate buffer.

As mentioned above, the basic protocol for this assay is described in Sasakawa et al., Int. Arch. Allergy Immunol., 126(3): 239-247 (2001) and Matsuoka et al., Allergy, 58(2): 139-145 (2003).

Method

Mice were treated with either a formulation containing 0.25% NF-κB decoy molecules, formulation alone without any decoy molecules or topical betamethasone for 17 days. Right ears of Dp-injected mice were removed 1 day after the final decoy treatment. Part of the ear was fixed in 10% phosphate buffered formalin (pH 7.2) and embedded in paraffin, and 3 micron sections were cut. The samples were stained with hematoxylin and eosin for histology and toluidine blue for detection of degranulated mast cells.

Results and Analysis

Histological examination of the skin lesions was performed on day 17 of treatment. The hematoxylin and eosin staining showed severe epidermal hyperplasia and cellular infiltration into the dermis of vehicle treated ears injected with Dp, and treatment with NF-κB decoy produced both a decrease in epidermal hyperplasia and cellular infiltrate. Most of the mast cells in the Dp-injected, vehicle treated mice were degranulated while treatment with NF-κB decoy molecule demonstrated a decrease in degranulated mast cells.

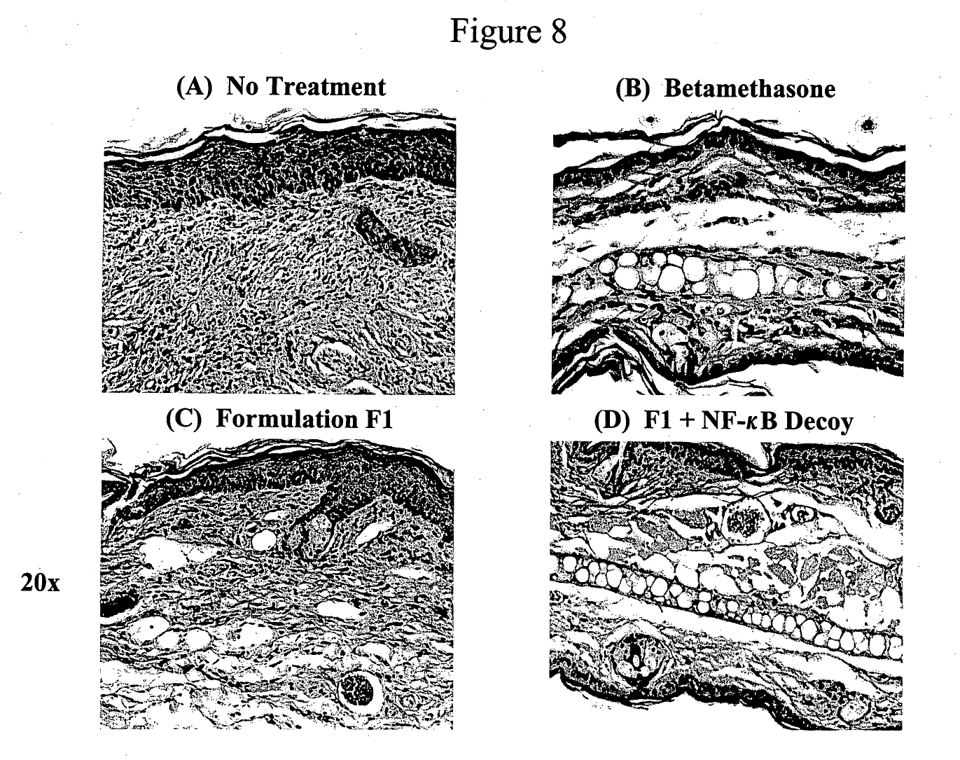

FIG. 8 shows hematoxylin and eosin staining of formalin-fixed mouse skin with atopic dermatitis that (A) received no treatment, (B) was treated with betamethasone, (C) was treated with a formulation F1 containing about 49% ethanol by weight and about 0.8% sodium laureth sulfate by weight and (D) was treated with a formulation F1 containing about 49% ethanol by weight and about 0.8% sodium laureth sulfate by weight containing the NF-κB decoy molecules.

These data indicate that topical application of NF-κB decoy suppresses inflammation cause by injection of Dp into the ear. Accordingly, NF-κB decoy treatment decreases epidermal hyperproliferation, cellular infiltration and degranulation of mast cells.

### Example 7

### Effects NF-κB Decoy Molecules—Murine Model

This example illustrates the adverse side effects from the betamethasone treatment and lack thereof from the treatment by NF-κB decoy molecules in dustmite Ag (Dp) induced contact dermatitis in NC/Nga mice.

Dustmite antigen induced atopic dermatitis was induced as previously described above (Sasakawa, et al., 2001). Briefly, six week old male NC/Nga mice were injected intradermally with 5 mg of Dp extract (Greer Laboratories, Lenoir, N.C.) dissolved in saline on the ventral side of their right ears on days 0, 2, 4, 7, 9, and 11. Starting on day 11, the Dp injected ear was topically treated 2 times a day for 11 days with 20 μl of formulation F6, F6 containing 0.25% or 0.1% NF-κB decoy or topical 0.1% betamethasone valerate as the positive control. The ear thickness was measured with an ear thickness gauge (Oditest, Dyer Co., Lancaster, Calif.) 24 hours after each intradermal injection or treatment.

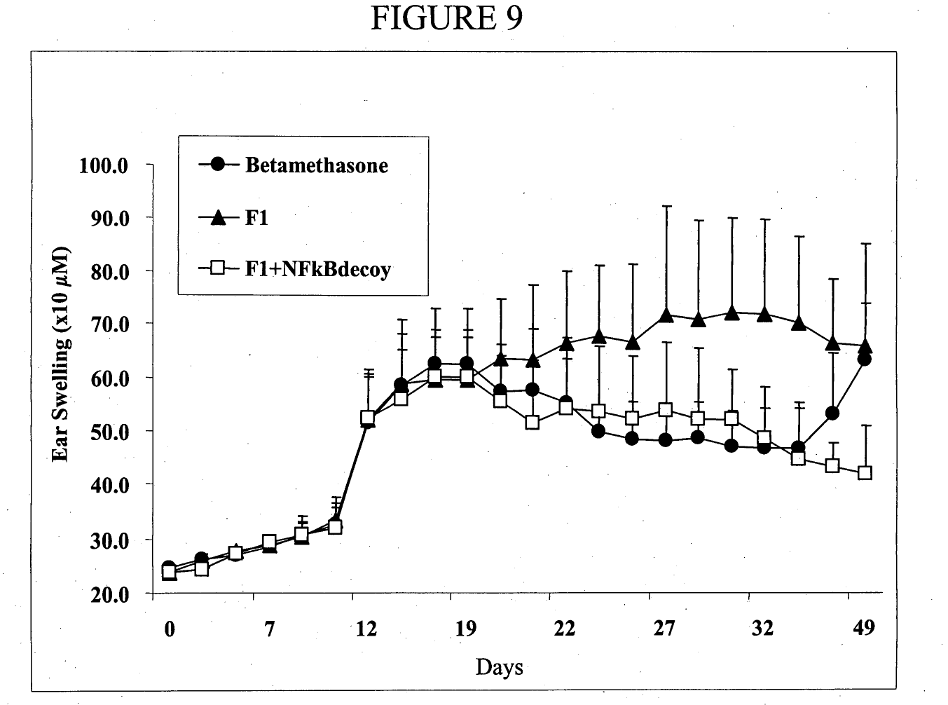

FIG. 9 shows that cessation of betamethasone resulted in a sudden and severe rebound of swelling and inflammation. However, the therapeutic benefit observed with NF-κB decoy molecules was maintained for 15 days after the treatment was terminated.

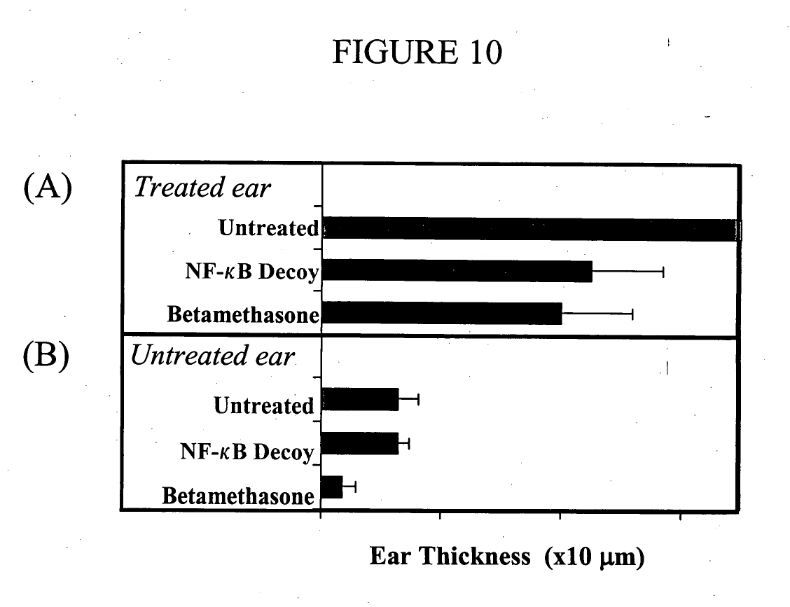

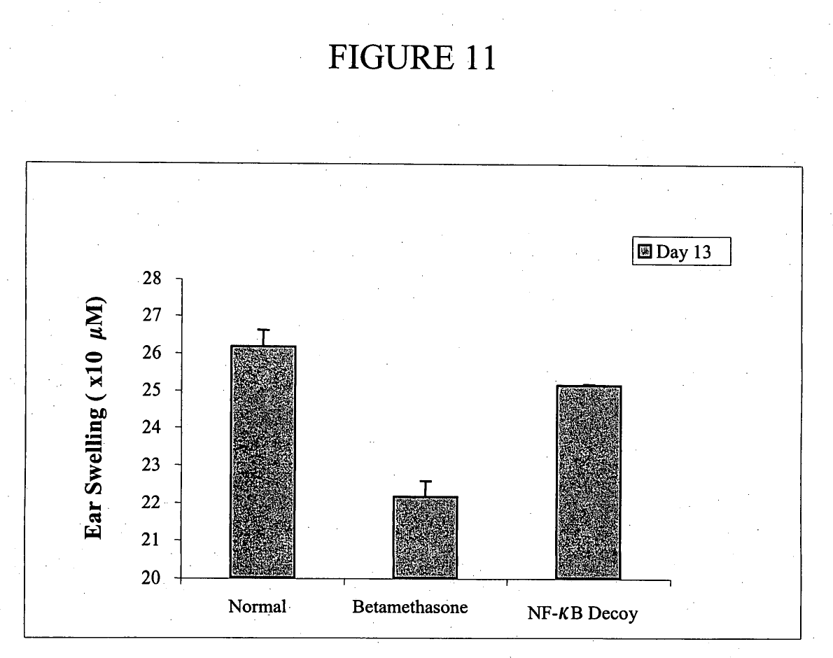

Furthermore, FIG. 10 shows that unlike betamethasone that induces skin atrophy within 4 days of treatment, prolong NF-κB decoy application fails to show any such side effects. Treated ears in FIG. 10(A) are the dustmite injected/inflamed ears. Due to the inflammation, the ear thickness is increased in the dustmite treated ears. In contrast, the untreated ears in FIG. 10(B) are the normal contralateral ears. FIG. 10(B) shows that when betamethasone is applied to the inflamed ear, it causes thinning of the untreated ear and shows systemic side effects of skin atrophy.

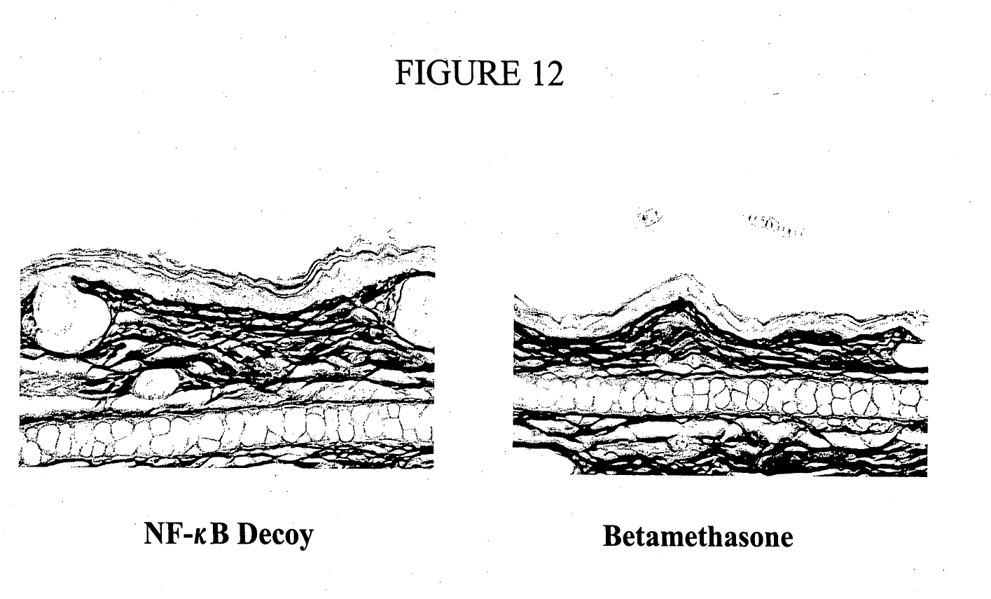

FIG. 11 further illustrates the side effect of betamethasone treatment on normal ears. FIG. 11 shows the measurement of the ear thickness of normal ears without any treatment, treatment with betamethasone and treatment with NF-κB decoy molecules. The betamethasone and NF-κB decoy molecules were administered twice a day and the measurement was taken at day 13 of the treatment.

FIGS. 10 and 11 clearly shows that topical steroids, such as betamethasone cause thinning of the skin while prolong treatment with NF-κB decoy molecules does not show similar adverse side effect.

For additional experiment, collagen was stained with picro-sirius red. Formalin-fixed, paraffin-embedded tissue samples were used to stain collagen using sirius red F3B dye (Puchtler et al., Beitrag für Pathologie, 150: 174-187 (1973); Junqueira et al., Histochem J., 11: 447-455 (1979)). In the dermis this stain specifically detects collagen type III (collagen type IV in the epidermal basement membrane does not stain). The laboratory skin sections are stained with Alcian blue pH=2.5, to delineate cartilage prior to picro-sirius red staining (Kiernan, J. A., Histological & Histochemical Methods: Theory and Practice. 3^{rd} Ed., Butterworth-Heinemann, Oxford (1999)).

Following deparaffinization and rehydration sections are stained with Alcian blue, pH=2.5, 30 minutes (BioCare Medical, Walnut Creek, Calif.) then washed in running DI water. Sections were stained overnight with picro-sirius red (0.1% sirius red F3B (CI35782, Sigma Chem. Co., St. Louis, Mo.) in saturated aqueous solution of picric acid (EMD Chemicals, Gibbstown, N.J.), followed by washing in running DI water, dehydration in ethanol, clearing in xylenes and coverslipping.

The slides were analyzed with an Axioskop 2 microscope, (Carl Zeiss AG, Gottingen, Germany), using 20× PlanApo objective, with brightfield illumination. Digital images were collected using NIKON DXM1200F digital camera (Technical Instruments, Burlingame, Calif.) and assembled in ADOBE® PHOTOSHOPS (Adobe, San Jose, Calif.).

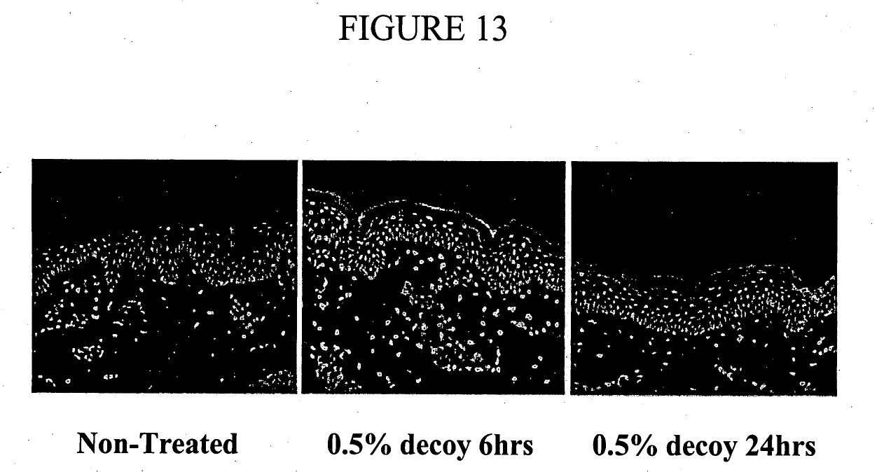

The results are shown in FIG. 12. FIG. 12 shows the side effect of betamethasone treatment on the dermal thickness in normal (non-inflamed) contralateral ears. FIG. 12 shows that there is no loss of collagen when the normal ears were treated with 0.25% NF-κB decoy molecules, when administered using an aqueous gel-based formulation F6 of the present invention. On the contrary, FIG. 12 shows the thinning of dermal thickness in the normal ears treated with betamethasone. The dermal thickness in the NF-κB decoy treated ear was 102±17 μm and in the betamethasone treated ear was 67±12 μm.

### Example 8

### Delivery NF-κB Decoy Molecules to Pig Skin

This example illustrates the efficient delivery of NF-κB decoy molecules in a porcine model. Due to similarities with structural elements and percutaneous absorption to human skin, porcine skin is an ideal model for testing drug delivery and penetration.

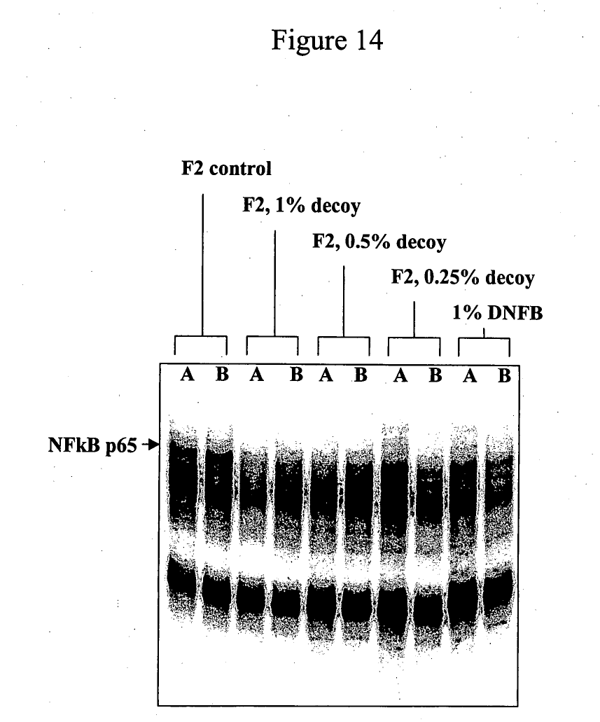

In this experiment, 0.5% of biotinylated NF-κB decoy in aqueous gel-based formulation F2 was applied to 30 μl/cm² area on the back of a light skinned female Yorkshire pig, (70-80 kg) for 24 hours. Skin was washed thoroughly with PBS to remove any reminiscing formulation. Skin was frozen in embedding medium. Cryosections of the skin were stained for decoy localization at the site of application. The biotinylated NF-κB decoy in cross sections was visualized using Alexa-tagged streptavidin (Molecular Probes, Eugene, Oreg.) and counterstained with Hoechst stain (Molecular Probes, Eugene, Oreg.) for nuclear colocalization. Fluorescent images were captured using color digital camera (SPOT Digital Camera System, Diagnostic Instruments, Inc., Sterling Heights, Mich.). FIG. 13 shows that the treatment of normal porcine skin with NF-κB decoy resulted in efficient nuclear localization of keratinocytes and stroma.

### Example 9

### Aqueous Formulation Containing NF-κB Decoy Molecule—Porcine Model

This example illustrates the efficacy of the formulations of the present invention for delivering NF-κB decoy molecules using a pig skin inflammation model. In this example, the drug penetration in dinitrofluorobenzene inflamed porcine skin was examined.

The aqueous gel-based formulation, F2, used in this experiment contained 0.8% sodium laureth sulfate, 10% ethanol, 2.0% HPMC 4000 cps and 10% 100 mM phosphate buffer.

DNFB-induced Delayed Hypersensitivity

A hypersensitivity to dinitrofluorobenzene (DNFB) exposure is induced in light skinned female Yorkshire pigs, 70-80 kg by applying 100 μl of a 10% (W/V) DNFB in 50% acetone/10% dimethylsulfoxide (DMSO)/30% olive oil mixture to the medial aspect of each auricle and to the groin on day 1. The animal is again exposed to 100 μl of a mixture of 2% DNFB in 58% acetone/10% dimethylsulfoxide (DMSO)/30% olive oil on the lateral aspects of both auricles on day 4. At defined intervals prior to a final DNFB challenge, a topical control formulation, or the same formulation containing the NF-κB decoy molecules are applied with an even coverage of 25 mg per cm to defined areas of 4 cm² on the dorsal skin of the sensitized animal, and maintained for an interval after which the residual formulation is removed using a water moistened cotton applicator. The DNFB challenge consists of the application of a mixture of DNFB ranging from 1% to 2.5%, as indicated in the results, in 69% acetone/30% olive oil on day 12, applied with an even coverage of 6.4 μl per cm² to the treated and control regions of the skin. At a time of 24 hour following the application of the DNFB challenge, the region of interest is evaluated for erythema and edema, after which a collection of 4 punch biopsies, 6 mm in diameter are recovered from the area of interest. The biopsy specimens are preserved for histological evaluation or divided into two halves and frozen on dry ice for use in gel-shift analysis or mRNA transcript analysis.

Electrophoretic Mobility Shift Assay (EMSA) and Analysis

The NF-κB decoy molecules present in the biopsy specimens from the above described experiment can be conveniently tested and characterized in a gel shift, or electrophoretic mobility shift assay (EMSA). This assay provides a rapid and sensitive method for detecting the binding of transcription factors to DNA. The assay is based on the observation that complexes of protein and DNA migrate through a non-denaturing polyacryamide gel more slowly than free double-stranded oligonucleotides. The gel shift assay is performed by incubating a purified protein, or a complex mixture of proteins (such as nuclear extracts), with a ³²p end-labeled DNA fragment containing a transcription factor-binding site. The reaction products are then analyzed on a non-denaturing polyacrylamide gel. The specificity of the transcription factor for the binding site is established by competition experiments, using excess amounts of oligonucleotides either containing a binding site for the protein of interest or a scrambled DNA sequence.

One-half of a 6 mm biopsy sample from above is pulverized to a fine powder under liquid nitrogen, followed by extraction and isolation of the nuclear proteins using the NE-PER® Nuclear and Cytoplasmic Extraction Kit (Pierce Biotechnology Inc., Rockford, Ill.) as per kit instructions. A volume of nuclear extract containing 5 μg of protein is incubated with 1 μl of 1 mg/ml poly dIdC, 1 μl of P³²-labeled oligonucleotide probe (35 finoles), 100 mM KCl, 10 mM Tris buffer, pH 8.0, 5 mM MgCl₂, 6% glycerol, 2 mM dithiothreitol, 2.5% bovine serum albumin, and 0.1% NP-40 in a total volume of 20 μl for 30 minutes at room temperature, then run on a 6% non-denaturing polyacrylamide gel. The gel is dried under vacuum at 80° C., then exposed to a phosphor-imaging plate followed by scanning on a Molecular Dynamics TYPHOON™ 8600 imager (Molecular Dynamics, Sunnyvale, Calif.).

The presence of NF-κB-bound decoy oligonucleotide in the tissue of interest is determined by the competitive reduction of the binding of the P³²-labeled probe to the p65 protein of NF-κB on the gel image.

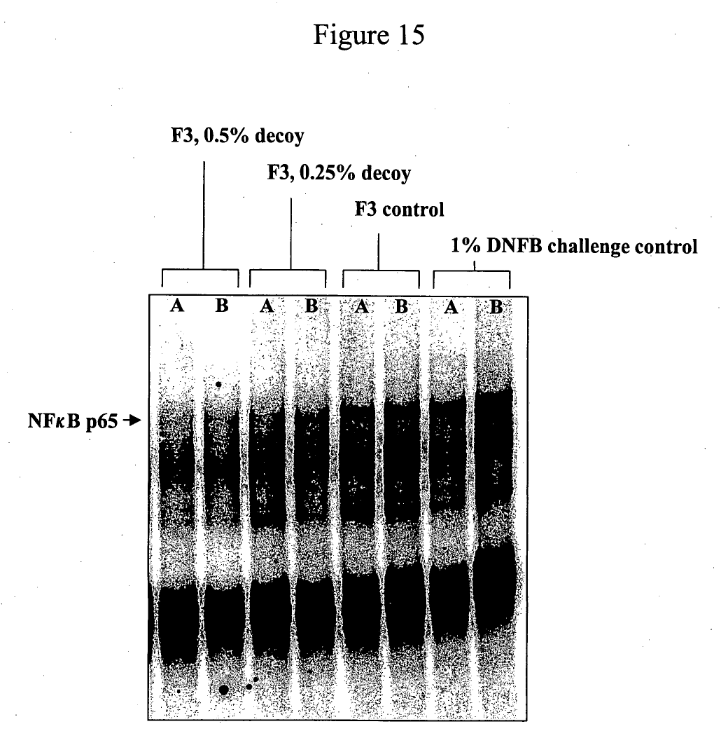

FIG. 14 shows quantitated results from competitive binding gel-shift assay which indicates the presence of NF-κB-bound decoy molecules in the pig skin that were treated with aqueous gel-based formulation F2 with 10% ethanol and varying concentration of NF-κB decoy molecules ranging from 0.25 to 1%. The P³²-labeled oligonucleotide probe was radiolabeled in this assay. The amount of band remaining after addition of competitor is graphed. Bands were quantitated using a TYPHOON™ 8600 Phosphorimager (Molecular Dynamics, Sunnyvale, Calif.). The presence of NF-κB-bound decoy molecules in the pig skin is determined by the reduction of the binding of the p³²-labeled oligonucleotide probe to the p65 protein on the gel image.

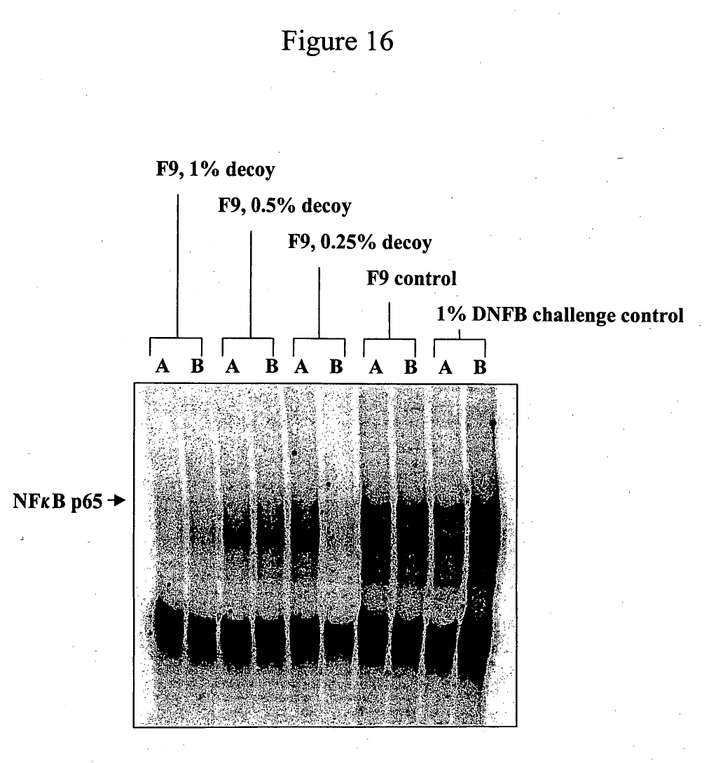

FIG. 15 shows quantitated results from competitive binding gel-shift assay which indicates the presence of NF-κB-bound decoy molecules in the pig skin that were treated with aqueous gel-based formulations F3 having 5% ethanol and 0.25 or 0.5% of NF-κB decoy molecules. The formulation designated as F3 control contains no NF-κB decoy molecules. The P³²-labeled oligonucleotide probe was radiolabeled in this assay. The amount of band remaining after addition of competitor is graphed. Bands were quantitated using a TYPHOON™ 8600 Phosphorimager (Molecular Dynamics, Sunnyvale, Calif.). The presence of NF-κB-bound decoy molecules in the pig skin is determined by the reduction of the binding of the P³²-labeled oligonucleotide probe to the p65 protein on the gel image.

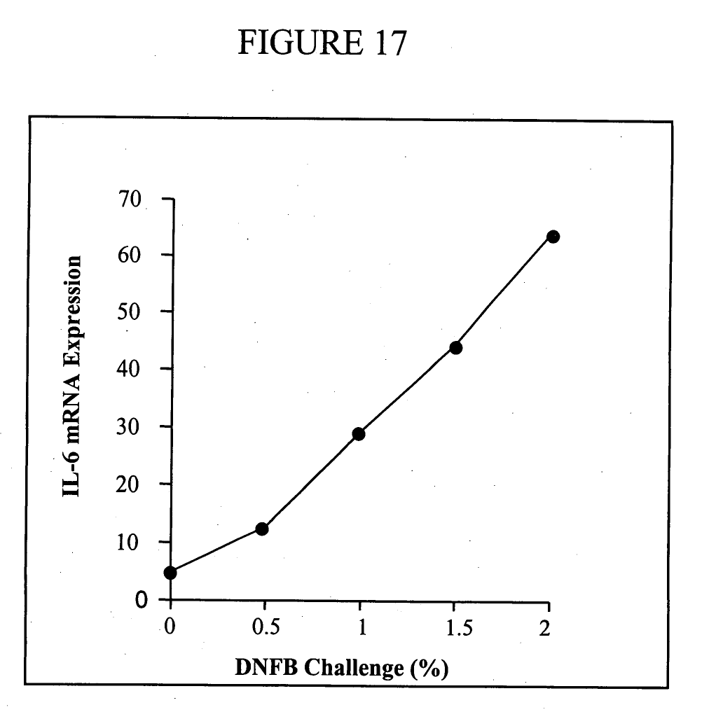

FIG. 16 shows quantitated results from competitive binding gel-shift assay which indicates the presence of NF-κB-bound decoy molecules in the pig skin that were treated with liposome-containing formulations F9 with varying concentration of NF-κB decoy molecules ranging from 0.25 to 1%. The formulation designated as F5 control contains no NF-κB decoy molecules. The P³²-labeled oligonucleotide probe was radiolabeled in this assay. The amount of band remaining after addition of competitor is graphed. Bands were quantitated using a TYPHOON™ 8600 Phosphorimager (Molecular Dynamics, Sunnyvale, Calif.). The presence of NF-κB-bound decoy molecules in the pig skin is determined by the reduction of the binding of the P³²-labeled oligonucleotide probe to the p65 protein on the gel image.

### Example 10

### Anti-Inflammatory Effects of NF-κB Decoy Molecule—Porcine Model

This example shows that NF-κB decoy molecules, when administered using an aqueous gel-based formulation of the present invention, effectively decrease the gene expression of the pro-inflammatory cytokines, such as IL-6 and IL-1β in dinitrofluorobenzene inflamed porcine skin.

A hypersensitivity to dinitrofluorobenzene (DNFB) exposure is induced in light skinned female Yorkshire pigs, 70-80 kg by applying 100 μl of a 10% (W/V) DNFB in 50% acetone/10% dimethylsulfoxide (DMSO)/30% olive oil mixture to the medial aspect of each auricle and to the groin on day 1. The animal is again exposed to 100 μl of a mixture of 2% DNFB in 58% acetone/10% dimethylsulfoxide (DMSO)/30% olive oil on the lateral aspects of both auricles on day 4. At defined intervals prior to a final DNFB challenge, a topical control formulation, or the same formulation containing the NF-κB decoy molecules are applied with an even coverage of 25 mg per cm to defined areas of 4 cm² on the dorsal skin of the sensitized animal, and maintained for an interval after which the residual formulation is removed using a water moistened cotton applicator. The DNFB challenge consists of the application of a mixture of DNFB ranging from 1% to 2.5%, as indicated in the results, in 69% acetone/30% olive oil on day 12, applied with an even coverage of 6.4 μl per cm² to the treated and control regions of the skin. At a time of 24 hour following the application of the DNFB challenge, the region of interest is collected. The biopsy specimens were paraffin embedded for histological hematoxylin and eosin staining and were frozen on dry ice for use in gel shift analysis of mRNA transcript analysis.

Part of the pig skin punch biopsy was flash frozen in liquid nitrogen and store at −80° C. Total RNA was isolated from the ears using QLκZOL® lysis reagent (Qiagen, Amtsgericht Düsseldorf, Germany) according to manufacturer's instruction. The expression of the mouse genes was assayed by real-time quantitative PCR with an BI PRISM® 7900 Sequence Detection System (Applied Biosystems, Foster City, Calif.). All procedures were carried out as previously described (Hurst et al., J. Immunol., 169(1):443-453 (Jul. 1, 2002)). Dnase-treated total RNAs were briefly mixed with random hexamers (Gibco-BRL, Carlsbad, Calif.), Oligo dt (Boehringer, Germany), and the first strand cDNAs were synthesized with SurperScript II™ reverse transcriptase (Gibco-BRL, Carlsbad, Calif.). Primers for the respective genes were designed using the primer design software PRIMER EXPRESS® (Applied Biosystems, Foster City, Calif.). Primers were synthesized at Sigma Genosys (Woodlands, Tex.). The quantitative PCR was performed using TAQMAN® PCR reagent kit (Applied Biosystems, Foster City, Calif.) according to the manufacturer's protocol. Sample cDNAs equivalent to 25 ng of RNA were examined in each reaction in a 384-well PCR plate. Levels of ubiquitin were measured for each sample, and used as internal standard. Cytokine levels are expressed as relative expression to ubiquitin levels.

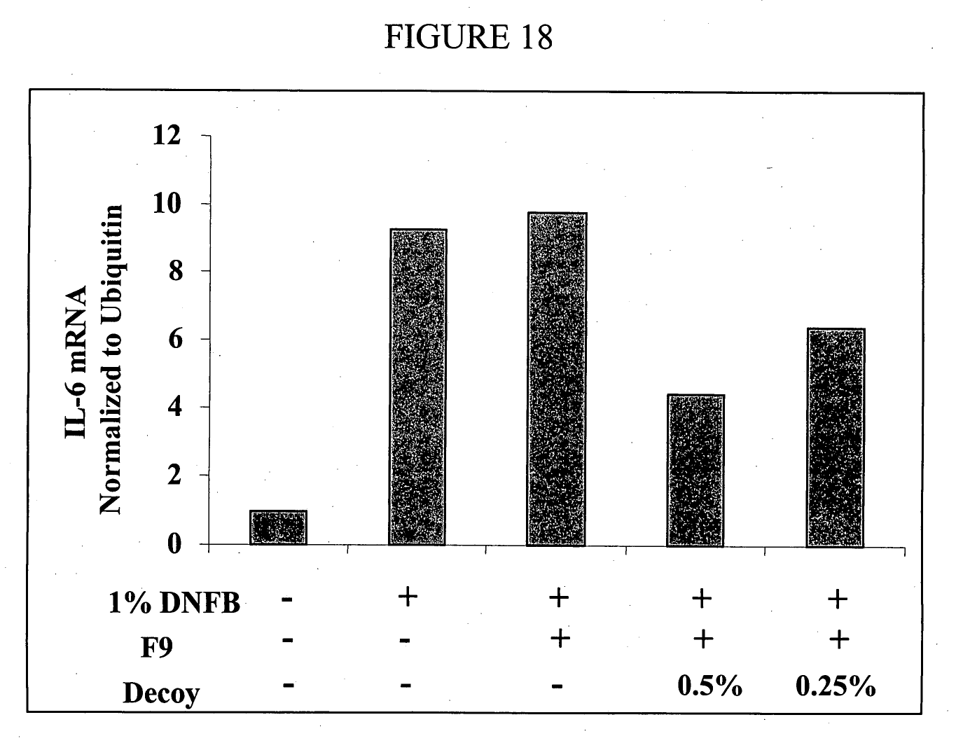

FIG. 17 is a graph showing the IL-6 mRNA expression levels in dinitrofluorobenzene inflamed porcine skin.

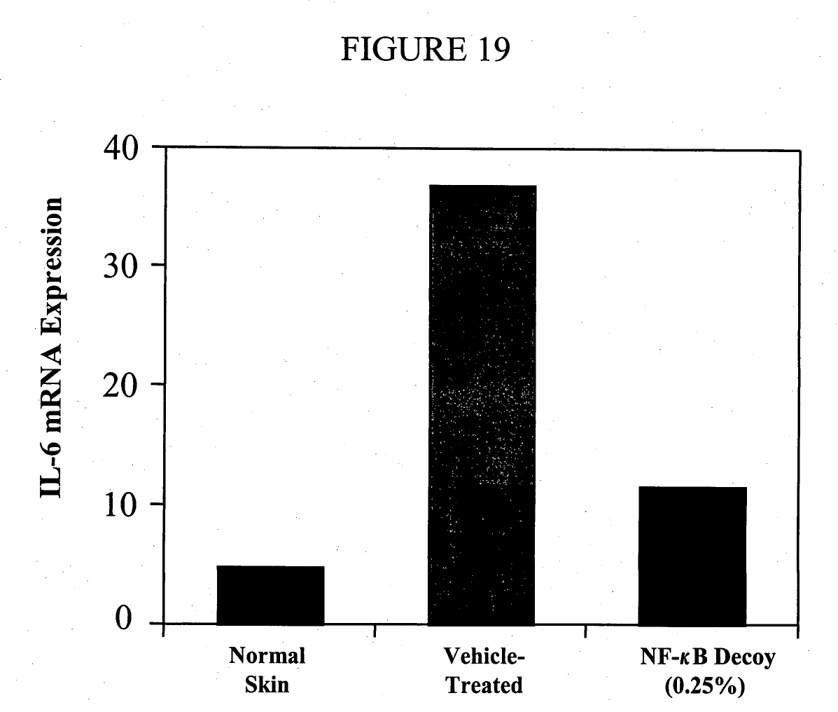

FIG. 18 is a graph showing the reduction in relative IL-6 mRNA expression levels in a pig skin treated with liposome-containing formulation F9 with 0.25 and 0.5% NF-κB decoy molecules when compared to placebo treated or untreated skin.

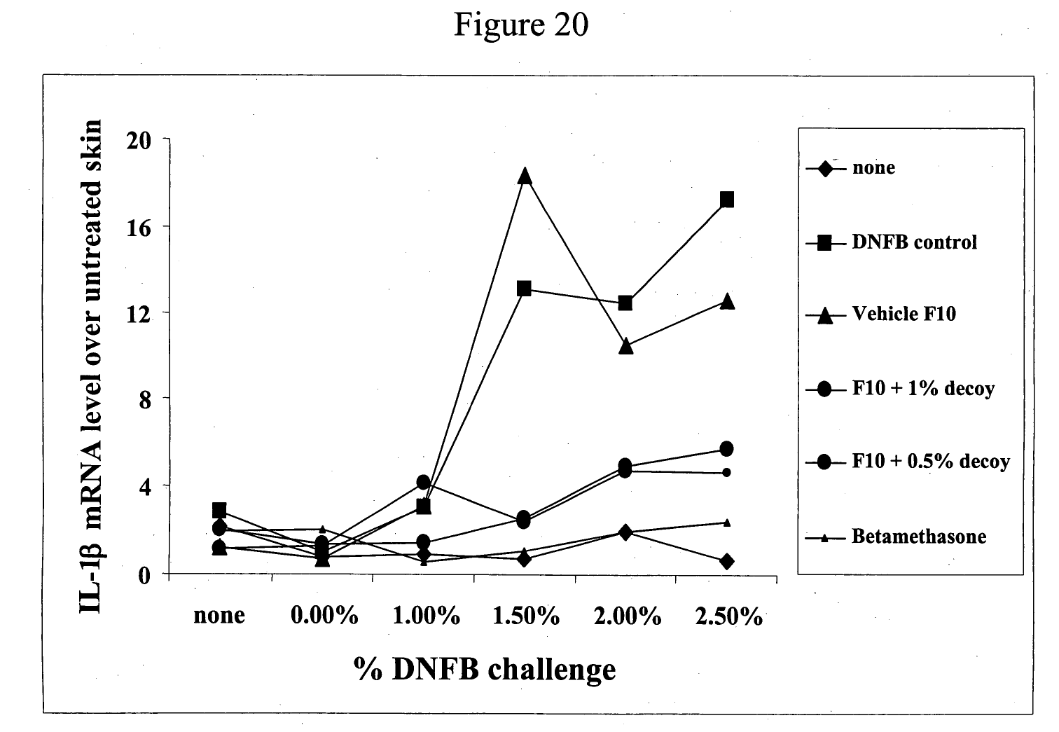

FIG. 19 is a graph showing the reduction of relative IL-6 mRNA expression levels in dinitrofluorobenzene inflamed porcine skin when treated with aqueous gel-based formulation F2 containing 0.25% NF-κB decoy molecules.

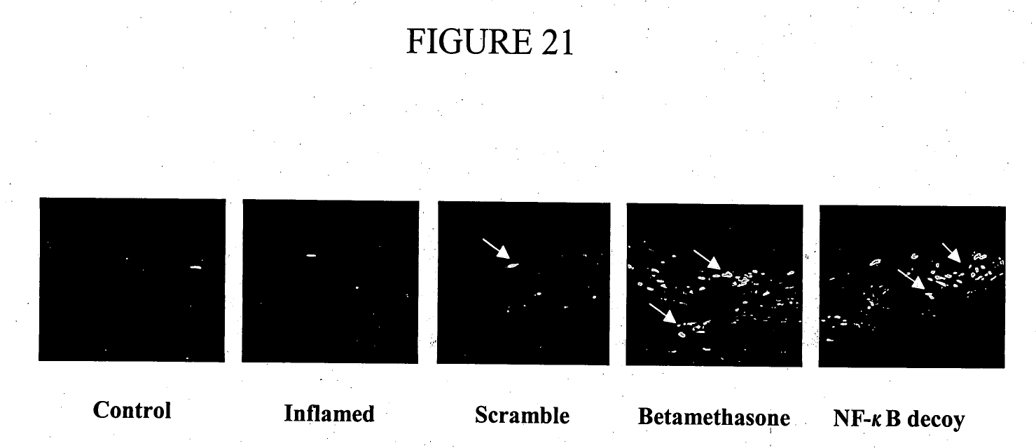

FIG. 20 shows the reduction of relative IL-1β mRNA expression levels in dinitrofluorobenzene inflamed porcine skin when treated with aqueous gel-based formulation F10 containing 0.5% or 1% NF-κB decoy molecules.

### Example 11

### Role of NF-κB Decoy Molecule in Reducing Inflammation

The purpose of this experiment was to determine the mechanism of NF-κB decoy in reducing inflammation in dustmite induced ear swelling in NC/Nga mice.

Dustmite antigen induced atopic dermatitis was induced as previously described above (Sasakawa, et al., 2001). Briefly, six-week old male NC/Nga mice were injected intradermally with 5 μg of Dp extract (Greer Laboratories) dissolved in saline. Injections were given on the ventral side of their right ears on days 0, 2, 4, 7, 9, and 11. Starting on day 11, the Dp-injected ear was topically treated two times a day for 2 days with 20 μl of aqueous gel-based formulation F6, aqueous gel-based formulation F6 containing 1% NF-κB decoy or 0.1% betamethasone valerate as a control. Tissues were formalin-fixed, paraffin-embedded tissue samples were used to identify apoptotic cells by the terminal deoxynucleotidyl transferase (TdT)-mediated dUTP nick end labeling (TUNNEL) assay (Gavrieli et al., J. Cell Biol., 119: 493-501 (1992)) with dUTP-FITC as a label. All the reagents were provided in the “In Situ Cell Death Detection Kit” (Roche, Indianapolis, Ind., Cat. No. 1 684 795). Following the TUNNEL reaction the sections were counterstained with 4′,6-diamidino-2-phenylindole, dihydrochloride (DAPI) (Molecular Probes, Eugene, Oreg.) and mounted with PROLONG GOLD® antifade reagent (Molecular Probes, Eugene, Oreg.).

Formalin-fixed, paraffin-embedded tissue samples were also used to identify proliferating cells using Ki67 rabbit monoclonal antibody (NeoMarkers Cat. No. RM-9106, Lab Vision Corp., Fremont, Calif.). Antigen retrieval was carried in citrate buffer, pH=6.1 (Target Retrieval Solution, Cat. No. S-1700, DakoCytomation, Carpinteria, Calif.), using the RETRIEVER®, a dedicated pressure cooker (EMS, Hatfield, Pa.). Following antigen retrieval Ki67 localization was done using a robotic immunostainer (DakoCytomation Autostainer, DakoCytomation, Carpinteria, Calif.). Endogenous peroxidase activity was inhibited with 3% H₂O₂. Non-specific binding of the immunoreagents was blocked using the following blocking solution: 4% BSA (Sigma Chem. Co., St. Louis, Mo.), 1% casein (EMD Chemicals, Gibbstown, N.J.), 0.5% teleostean fish skin gelatin (Sigma Chem. Co., St. Louis, Mo.) in TBS (50 mM Tris, pH=7.6, 500 mM NaCl, 0.05% Tween 20, all from Sigma Chem. Co., St. Louis, Mo.). Following blocking the sections were incubated with the Ki67 antibody (1 μg/ml final concentration) for 1 hour. Following washing the sections were incubated with anti-rabbit IgG ENVISION® reagent (1:1 dilution), labeled with peroxidase (DakoCytomation, Carpinteria, Calif.), for 30 min. Peroxidase activity was detected with DAB substrate (DAB⁺ reagent, DakoCytomation, Carpinteria, Calif.). Sections were thoroughly washed and coverslipped (without any counterstaining). The samples were analyzed with an Axioskop 2 epi-fluorescent microscope (Carl Zeiss AG, Gottingen Germany), using 20× NeoFluar objective. Digital images were collected using a SPOT RT™ camera (Diagnostic Instruments, Sterling Heights, Mich.) and assembled in ADOBE® PHOTOSHOP® (Adobe, San Jose, Calif.).

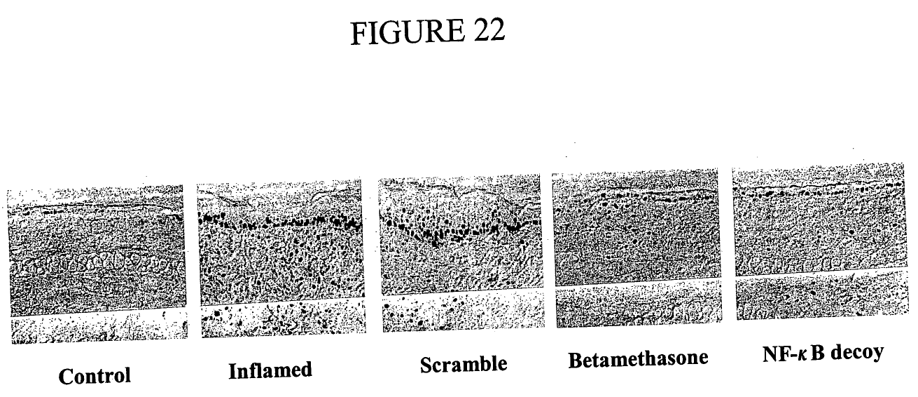

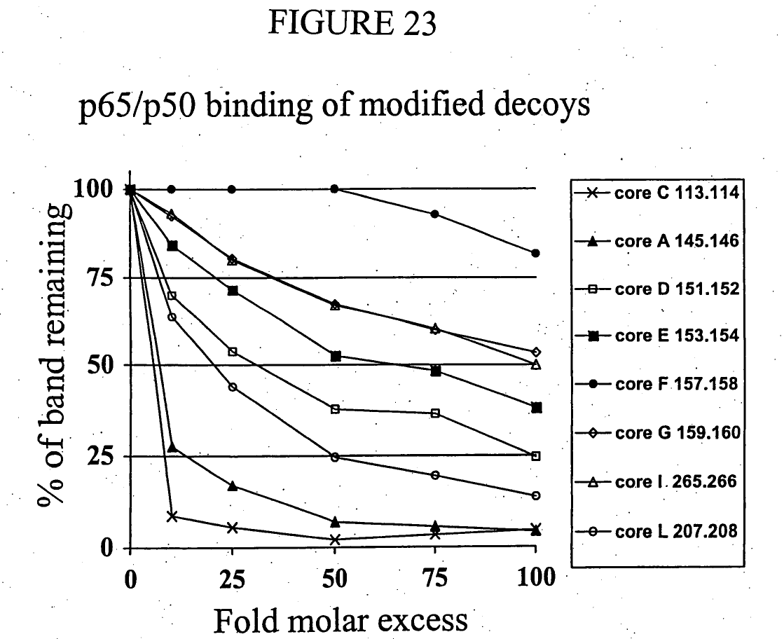

Since blockade of NF-κB function is associated with both induction of apoptosis as well as inhibition of proliferation, dustmite induced inflamed ears were analyzed for TUNNEL assay (FIG. 21) and Ki67 staining (FIG. 22). FIG. 21 shows that the enhanced apoptosis (green nuclei) mediated by betamethasone and NF-κB decoy application on inflamed ears is observed in both the epidermis and the dermal region of the skin. In contrast, the scramble decoy fail to elicit this response. Increased proliferation is detected with Ki67 staining (brown nuclei, FIG. 22) in the epidermal and dermal layers. Betamethasone and NF-κB decoy application on inflamed ears dramatically decrease the mitotic index, indicative of inhibition of proliferation in the inflamed ears. Scramble decoy did not show any change of Ki67 positive cells as compared to non-treated inflamed ears.

FIGS. 21 and 22 show that topical NF-κB decoy treatment results in increased apoptosis and decreased proliferation of inflammatory cells in Dp induced inflammation. Similar effects were seen with betamethasone treatment. These observations explain the strong and long-lasting anti-inflammatory effect of NF-κB decoy in the mouse atopic dermatitis-like model.

### Example 12

### NF-κB Decoy Molecules Design

NF-κB dsODN decoy molecules were designed and tested for their ability to bind and/or compete for binding of NF-κB. In a particular aspect, the goal of the invention was to design NF-κB decoy molecules that preferentially bind p65/p50 and/or cRel/p50 heterodimers over p50/p50 homodimers. As a result of not blocking p50/p50 homodimers, the selective decoy molecules of the invention allow these homodimers to block the promoters of NF-κB regulated genes, which provides an additional level of negative regulation of gene transcription.

In designing the oligonucleotide decoys, information available from crystal structure studies and computational analysis of the known NF-κB binding sites were utilized.

As discussed above, based on study of the crystal structure of the p50/p65 heterodimer bound to the immunoglobulin light-chain gene, which contains the consensus sequence of 5′-GGGACTTTCC-3′ (SEQ ID NO: 2), it has been shown that p50 contacts the 5-base-pair subsite 5′-GGGAC-3′ (SEQ ID NO: 3) and that p65 contacts the 4-base-pair subsite 5′TTCC-3′ (SEQ ID NO: 4). A series of NF-κB oligonucleotide decoys were designed, which contained fewer numbers of G's at the 5′ end of the consensus binding site with the aim to prepare decoy molecules that would have lower affinity for the p50/p50 homodimer but still bind the p65/p50 heterodimer. These oligonucleotide decoys were assigned “core” and “flank” letter codes for ease of identification and presentation. The cores were assigned letter codes “A” through “L” and the flanks “T” through “Z”. The decoys were tested in the gel shift assay to determine their ability to compete with a high affinity radiolabeled oligonucleotide for NF-κB binding. The NF-κB-binding DNA consensus sequences were selected from publications of NF-κB related DNA-protein interactions, including: Blank et al., EMBO J. 10:4159-4167 (1991); Bours et al. Mol. Cell. Biol. 12:685-695 (1992); Bours et al. U. Cell 72:729-739 (1993); Duckett et al. Mol. Cell. Biol. 13:1315-1322 (1993); Fan C. -M., Maniatis T., Nature 354:395-398 (1991); Fujita et al., Genes Dev. 6:775-787 (1992); Fujita et al. Genes Dev. 7:1354-1363 (1993); Ghosh et al., Nature 373:303-310 (1995); Ghosh et al., Cell 62:1019-1029 (1990); Grumont et al., Mol. Cell. Biol. 14:8460-8470 (1994); Henkel et al., Cell 68:1121-1133 (1992); Ikeda et al. Gene 138:193-196 (1994); Kunsch et al., Mol. Cell. Biol. 12:4412-4421 (1992); LeClair et al., Proc. Natl. Acad. Sci. USA 89:8145-8149 (1992); Li C. -C. et al., J. Biol. Chem. 269:30089-30092 (1994); Matthews et al., Nucleic Acids Res. 21:1727-1734 (1993); Mueller et al., Nature 373:311-317 (1995); Neri et al., Cell 67:1075-1087 (1991); Nolan et al., Cell 64:961 -969 (1991); Paya et al., Proc. Natl. Acad. Sci. USA 89:7826-7830 (1992); Plaksin et al., J. Exp. Med. 177:1651-1662 (1993); Schmid et al., Nature 352:733-736 (1991); Schmitz M. L., Baeuerle P. A. EMBO J. 10:3805-3817 (1991); Ten et al., EMBO J 11: 195-203 (1992); Toledano et al., J. Mol. Cell. Biol. 13:852-860 (1993); Urban et al., EMBO J 10:1817-1825 (1991).

Based on this available information, we generated a set of decoys for initial screening. These decoys include a “mutation decoy”, the scrambled decoys, decoys with different length at their 5′ or 3′ end, and decoys with alternative base composition within the core region and/or in the flanking sequences.

To better understand the base-composition near the core binding sites of NF-κB, the core binding sites were computationally aligned (forward strand only) with known binding sequences. Based on this alignment, several major groups of decoys with slightly different core binding sites were created.

The major core and flanking sequences are listed in Table 1.

TABLE 1

| | | | | | |
|---|---|---|---|---|---|
| LETTER | | SEQ | LETTER | | SEQ |
| CODE | CORE | ID | CODE | FLANK | ID |

| | | | | | |
|---|---|---|---|---|---|
| A | GGGACTTTCC | 5 | T | CCTTGAA . . . TCC | 6 |
| B | GGGGACTTTCC | 7 | U | AT . . . GT | 8 |
| C | GGGGACTTTCCC | 9 | V | TC . . . TC | 10 |
| D | GGGATTTCC | 11 | W | CTC . . . TGT | 12 |
| E | GGACTTTCC | 13 | W′ | CTC . . . TCA | 14 |
| F | GACTTTCC | 15 | X | CT . . . TC | 16 |
| G | GACTTTCCC | 17 | Y | TC . . . CA | 18 |
| H | GGATTTCC | 19 | Z | AGTTGA . . . AGGC | 20 |
| I | GGATTTCCC | 21 | Z′ | TTGA . . . AGGC | 22 |
| J | GATTTCC | 23 | | | |
| K | GATTTCCC | 24 | | | |
| L | GGACTTTCCG | 25 | | | |

Electrophoretic Mobility Shift Assay (EMSA)

The EMSA assay was employed to characterize oligonucleotide decoys for the NF-κB transcription factor. Using a radiolabeled oligonucleotide probe (non-mammalian, based on NF-κB promoter from HIV, sequence 113/114), which exhibits high affinity for relevant members of the NF-κB family, binding of p65/p50, cRel/p50 and p50/p50 was tested using a nuclear extract from an activated monocyte cell line. Using the above modified oligonucleotides to compete for binding for the above-mentioned NF-κB family members, it was possible to compare the binding affinity of these oligonucleotides against the high affinity radiolabeled probe and each other. This assay has also enabled the design of a decoy which selectively binds particular members of the NF-κB family. By using increasing concentrations of various oligonucleotides, it was observed that, by deleting or changing targeted residues in the binding site, it was possible to specifically decrease the binding of the decoy molecule to p50/p50 homodimers, while retaining the affinity for p65/p50 and cRel/p50 heterodimers.

The NF-κB gel shift assays (EMSA) were performed as follows. A double-stranded oligonucleotide containing a consensus NF-κB binding site (5′ AGTTGAGGGGACTTTCCCalif.GGC 3′) (SEQ ID NO: 26) was end-labeled with γ³²P-ATP using T4 Polynucleotide Kinase (Promega). One microgram of a nuclear extract prepared from LPS stimulated THP-1 cells (human monocyte cell line) was incubated with 35 fmol of radiolabeled probe in the presence or absence of competing unlabeled NF-κB double-stranded oligonucleotides (dsODN) or scrambled dsODN. The incubations were carried out at room temperature for 30 minutes in a 20 μl reaction volume composed of 10 mM Tris-HCl pH 8, 100 mM KCL, 5mM MgC12, 2mM DTT, 10% Glycerol, 0.1% NP-40, 0.025% BSA and 1 μg Poly-dIdC. The reactions were loaded onto a 6% polyacrylamide gel, subjected to electrophoresis and dried. The dried gels were imaged and quantitated using a Typhoon 8600 Phosphorlmager (Amersham) and ImageQuant software. The identity of the NF-κB proteins contained in complexes bound to the radiolabeled oligonucleotide probe were identified by pre-incubating the reactions for 5 minutes with individual antibodies specific for each member of the NF-κB family prior to the addition of the radiolabeled probe.

Nuclease Degradation and Chemistry Modifications

Native DNA is subject to rapid degradation inside of a cell, primarily through the action of 3′ exonucleases, but also as a result of endonuclease attack. Therefore, oligonucleotide decoys are designed, they are modified to enhance their stability. Replacing one of the non-bridging oxygen atoms of the internucleotide linkage with a sulfur group, creating what is referred to as a phosphorothioate (PS) oligodeoxynucleotide, has been highly successful. The molecules are relatively nuclease resistant; however, they have been shown to exhibit nonspecific protein binding relative to 3′-terminally modified and unmodified oligonucleotide decoys (Brown et al, J. Biol. Chem. 269(43):26801-5 (1994)). Therefore, we performed a set of experiments to determine how many sulfurs were required at the 3′, 5′ or an internal site to provide nuclease resistance to our oligonucleotide decoys while maintaining the achieved specificity.

The Analysis of the EMSA Results

As discussed earlier, one goal of the work disclosed herein has been to develop NF-κB oligonucleotide decoy molecules that preferentially bind to the NF-κB p65/p50 and/or cRel/p50 heterodimer relative to the p50/p50 homodimer. The experimental results showed that the binding to p65/p50 and cRel/p50 were generally equivalent, therefore, only the p65/p50 bands were quantitated in our analysis.

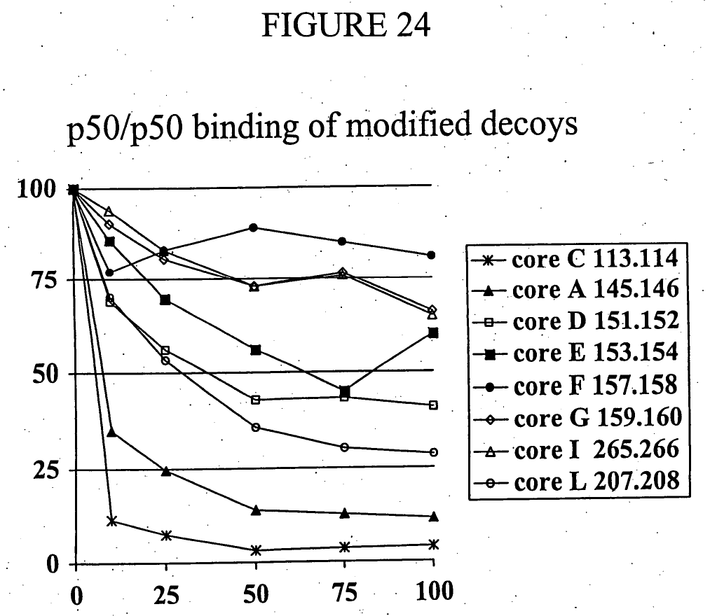

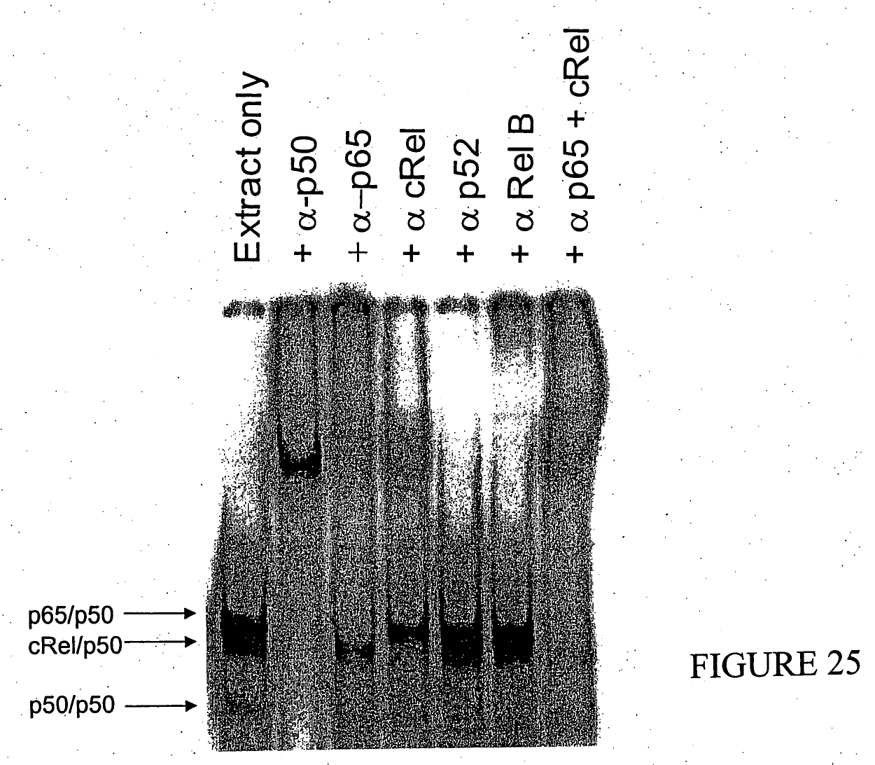

FIG. 23 shows the p65/p50 binding of certain NF-κB decoy molecules. FIG. 24 shows the p50/p50 binding of certain NF-κcB decoy molecules.

Preferential binding was quantified using the specificity/affinity factor, calculated as follows:
Specificy/affinity factor=(S_{p50/p50}−S_{p65/p50})×S_{p50/p50}/S_{p65/p50}

where S_{p50/p50} equals the molar excess of decoy required to compete 50% of the binding of p50/p50 to the non-mammalian NF-κB promoter from HIV (sequence 113/114) and S_{p65/p50} equals the molar excess of decoy required to compete 50% of the binding of p65/p50 to the non-mammalian NF-κB promoter from HIV (sequence 113/114, where the reverse strand corresponding to sequence 113 is designated as “114”). The score (S) is assigned as 100 if the decoy is unable to compete at least 50% of the binding at any molar ratio tested.

A preferred decoy molecule will have a lower score for the p65/p50 heterodimer and a higher score for the p50/p50 homodimer. The specificity of the decoy to p65/p50 heterodimer versus p50/p50 homodimer is proportional to their difference of score (score p50/p50—score p65/50). The results of the EMSA competition binding experiments, performed as described above, are summarized in Table 2, where the decoy molecules are listed starting with the most specific decoys (highest specificity/affinity factor).

TABLE 2

| | | | | | | | | |
|---|---|---|---|---|---|---|---|---|
| | | Core- | | | (p50- | (p50- | Specificity/ | SEQ |
| | | Flank | | | p65)* | p65)/ | affinity | ID |
| ID | Sequences | Alias | p65 | P50 | p50 | p65 | factor | NO. |

| | | | | | | | | |
|---|---|---|---|---|---|---|---|---|
| 173 | TTGAGGACTT | E-Z-4 | 55 | 100 | 4500 | 0.82 | 81.82 | 26 |
| | TCGAG | | | | | | | |
| 177 | CTCGGAGTTT | E-W | 57.5 | 100 | 4250 | 0.74 | 73.9 | 27 |
| | CCTGT | | | | | | | |
| 151 | AGTTGAGGGA | D-Z | 36 | 69 | 2277 | 0.92 | 63.25 | 28 |
| | TTTCCAGGC | | | | | | | |
| 207 | AGTTGAGGAC | L-Z | 20 | 45 | 1125 | 1.25 | 56.25 | 29 |
| | TTTCCCAGGG | | | | | | | |
| 153 | AGTTGAGGAC | E-Z | 62.4 | 96 | 3225.6 | 0.54 | 51.69 | 30 |
| | TTTCCAGGC | | | | | | | |
| 265 | AGTTGAGGAT | I-Z | 54 | 84 | 2520 | 0.56 | 46.67 | 31 |
| | TTCCCAGGC | | | | | | | |
| 235 | TCGGACTTTC | L-V | 37 | 62.5 | 1593.75 | 0.69 | 43.07 | 32 |
| | CCTC | | | | | | | |
| 117 | ATGGACTTTC | E-U | 72.5 | 100 | 2750 | 0.38 | 37.93 | 33 |
| | CGT | | | | | | | |
| 227 | TCGGATTTCC | H-V | 74 | 100 | 2600 | 0.35 | 35.14 | 34 |
| | TC | | | | | | | |
| 155 | TCGGACTTTC | E-V | 81 | 97 | 1552 | 0.20 | 19.16 | 35 |
| | CTC | | | | | | | |
| 281 | TTGAGGACTT | E-Z | 87 | 100 | 1300 | 0.15 | 14.94 | 36 |
| | TCCAGGC | EVEN | | | | | | |
| 121 | TCGGGACTTT | A-V | 25 | 34 | 306 | 0.36 | 12.24 | 37 |
| | CCTC | | | | | | | |
| 263 | AGTTGAGGAT | H-Z | 73 | 82 | 738 | 0.12 | 10.11 | 38 |
| | TTCCAGGC | | | | | | | |
| 295 | TGAGGACTTT | | 92 | 100 | 800 | 0.09 | 8.70 | 39 |
| | CCAGGCTC | | | | | | | |
| 289 | TGAGGACTTT | | 93 | 100 | 700 | 0.08 | 7.53 | 40 |
| | CCAGGC | | | | | | | |
| 279 | TTGCGGACTT | E-Z | 48 | 52 | 208 | 0.08 | 4.33 | 41 |
| | TCGAGGG | A→C | | | | | | |
| | | EVEN | | | | | | |
| 191 | CTGGGACTTT | A-X | 19 | 22.5 | 78.75 | 0.18 | 4.14 | 42 |
| | CCTC | | | | | | | |
| 141 | GTTGAGGGAC | A-Z-2 | 7.5 | 10 | 25 | 0.33 | 3.33 | 43 |
| | TTTCCAGG | | | | | | | |
| 123 | CTCGGGACTT | A-W | 8 | 10 | 20 | 0.25 | 2.50 | 44 |
| | TCCTGT | | | | | | | |
| 195 | TCGGGGACTT | C-V | 8.5 | 9 | 4.5 | 0.06 | 0.53 | 45 |
| | TCCCTC | | | | | | | |
| 103 | CAGTAGTATG | | 100 | 100 | 0 | 0.00 | 0.00 | 46 |
| | TGAGCCTGC | | | | | | | |
| 107 | TTGCCGTACC | SCRAM | 100 | 100 | 0 | 0.00 | 0.00 | 47 |
| | TGACTTAGCC | BLED | | | | | | |
| 113 | AGTTGAGGGG | C-Z | 5 | 5 | 0 | 0.00 | 0.00 | 48 |
| | ACTTTCCCAG | | | | | | | |
| | GC | | | | | | | |
| 129 | TCGGGATTTG | D-V | 37.5 | 37.5 | 0 | 0.00 | 0.00 | 49 |
| | CTC | | | | | | | |
| 145 | AGTTGAGGGA | A-Z | 8 | 8 | 0 | 0.00 | 0.00 | 50 |
| | CTTTCCAGGC | | | | | | | |
| 157 | AGTTGAGACT | F-Z | 100 | 100 | 0 | 0.00 | 0.00 | 51 |
| | TTCCAGGC | | | | | | | |
| 159 | AGTTGAGACT | G-Z | 100 | 100 | 0 | 0.00 | 0.00 | 52 |
| | TTCGCAGGC | | | | | | | |
| 169 | GGACTTTCC | E | 100 | 100 | 0 | 0.00 | 0.00 | 53 |
| 171 | AGGACTTTCC | E-A | 100 | 100 | 0 | 0.00 | 0.00 | 54 |
| | A | FLANK | | | | | | |
| 179 | CTGGACTTTC | E-X | 100 | 100 | 0 | 0.00 | 0.00 | 55 |
| | CTC | | | | | | | |
| 183 | AAGAGGACTT | E-AG | 100 | 100 | 0 | 0.00 | 0.00 | 56 |
| | TCCAGAG | FLANK | | | | | | |
| 185 | ATATGGACTT | E-AT | 100 | 100 | 0 | 0.00 | 0.00 | 57 |
| | TCCTTAA | FLANK | | | | | | |
| 187 | CAACGGACTT | E-CA | 100 | 100 | 0 | 0.00 | 0.00 | 58 |
| | TCCACAC | FLANK | | | | | | |
| 189 | CAGTGGACTT | E-CAGT | 100 | 100 | 0 | 0.00 | 0.00 | 59 |
| | TCCACTG | FLANK | | | | | | |
| 213 | TCGACTTTCC | G-V | 100 | 100 | 0 | 0.00 | 0.00 | 60 |
| | CTC | | | | | | | |
| 221 | CTGGGGACTT | C-X | 25 | 25 | 0 | 0.00 | 0.00 | 61 |
| | TCCCTC | | | | | | | |
| 229 | TCGGATTTCC | I-V | 100 | 100 | 0 | 0.00 | 0.00 | 62 |
| | CTC | | | | | | | |
| 231 | TCGATTTCCT | J-V | 100 | 100 | 0 | 0.00 | 0.00 | 63 |
| | C | | | | | | | |
| 233 | TCGATTTCCC | K-V | 100 | 100 | 0 | 0.00 | 0.00 | 64 |
| | TC | | | | | | | |
| 239 | CTCGGGGACT | C-W′ | 9 | 9 | 0 | 0.00 | 0.00 | 65 |
| | TTCCCTCA | | | | | | | |
| 241 | CTCGGAGTTT | E-W′ | 100 | 100 | 0 | 0.00 | 0.00 | 66 |
| | CCTCA | | | | | | | |
| 273 | TTGAGGATTT | H-Z′ | 100 | 100 | 0 | 0.00 | 0.00 | 67 |
| | CCAGGC | (−3′2BP) | | | | | | |
| 275 | TTGAGGATTT | H-Z′ | 100 | 100 | 0 | 0.00 | 0.00 | 68 |
| | CCAGGCT | (−3′1BP) | | | | | | |
| 277 | TTGAGGATTT | H-Z′ | 100 | 100 | 0 | 0.00 | 0.00 | 69 |
| | CCAGGCTC | | | | | | | |
| 283 | TGAGGACTTT | E-Z-3 | 100 | 100 | 0 | 0.00 | 0.00 | 70 |
| | CCAGG | | | | | | | |
| 285 | GAGGACTTTC | E-Z-6 | 100 | 100 | 0 | 0.00 | 0.00 | 71 |
| | GAG | | | | | | | |
| 287 | GTTGAGGACT | | 100 | 100 | 0 | 0.00 | 0.00 | 72 |
| | TTCCAGGC | | | | | | | |
| 291 | GAGGACTTTC | | 100 | 100 | 0 | 0.00 | 0.00 | 73 |
| | CAGGC | | | | | | | |
| 293 | AGGACTTTCC | | 100 | 100 | 0 | 0.00 | 0.00 | 74 |
| | AGGC | | | | | | | |
| 297 | AGGACTTTCC | | 100 | 100 | 0 | 0.00 | 0.00 | 75 |
| | AGGCTC | | | | | | | |
| 259 | TTGAGGACTT | E-Z′ | 87 | 85 | -170 | -0.02 | -1.95 | 76 |
| | TCCAGGCTC | | | | | | | |
| 197 | CTCGGGGACT | C-W | 26 | 17.5 | -148.75 | -0.33 | -5.72 | 77 |
| | TTCCCTGT | | | | | | | |

E-Z minus 4 is E-Z with the two 5′ and 3′ bases deleted
E-Z even is E-Z minus the two 5′ bases
E-Z a to C Even is E-Z with the A in position 6 changed to a C
A-Z-2 is A-Z with the 5′ and 3′ bases deleted
E-A is the E core with an A added to the 5′ and 3′ ends
E-AG Flank is E core with AAGA as the 5′ flank and AGAG as the 3′ flank
E-AT Flank is E core with ATAT as the 5′ flank and TTAA as the 3′ flank
E-CA Flank is E core with CAAC as the 5′ flank and ACAC as the 3′ flank
E-CAGT Flank is E core with CAGT as the 5′ flank and ACTG as the 3′ flank
H-Z′ (−3′2BP) is H-Z with the two 5′ bases deleted
H-Z′ (−3′1 BP) is H-Z with two 5′ bases deleted and a T added at the 3′ end
E-Z-3 is E-Z with AGT deleted from the 5′ end with C deleted from the 3′ end
E-Z-6 is E-Z with AGTT deleted from the 5′ end and GC deleted from the 3′ end

The data set forth in Table 2 suggest that the decoys with better p65/p50 ficity most likely share the “E” or “D” or “H” core sequence and “Z” or “W” or “V” or flanking sequences. In a more preferred group, the core sequence is “E” or “D” and the flanking sequence is “Z.” The decoy designated 153/154 was chosen as best from the top few candidates with the consideration of other parameters (see below).

The Analysis of Chemical Modification of DNA Backbone

A similar analysis was applied to evaluate the chemical modification of DNA backbone for tested decoys.

TABLE 3
S_{p50/p50}/S_{p65/p50} and specificy/affinity factor for 153/154 with various DNA backbones

| | | | | | | Specificity/Affinity |
|---|---|---|---|---|---|---|
| backbone | p65 | p50 | p50 − p65 | (p50 − p65) * p50 | (p50 − p65)/p65 | Factor |
| PO/H11 | 7.5 | 40 | 32.5 | 1300 | 4.33 | 173.33 |
| H7/H7 | 39 | 100 | 61 | 6100 | 1.56 | 156.41 |
| H6/H6 | 40 | 100 | 60 | 6000 | 1.50 | 150.00 |
| H3/H3 | 45 | 100 | 55 | 5500 | 1.22 | 122.22 |
| H11/PO | 7.5 | 33 | 25.5 | 841.5 | 3.40 | 112.20 |
| H5/H5 | 50 | 100 | 50 | 5000 | 1.00 | 100.00 |
| PO | 58 | 92 | 34 | 3128 | 0.59 | 53.93 |
| PO/H5 | 65 | 100 | 35 | 3500 | 0.54 | 53.85 |
| H8/H8 | 29 | 53 | 24 | 1272 | 0.83 | 43.86 |
| H10/H10 | 21 | 42 | 21 | 882 | 1.00 | 42.00 |
| H5/PO | 59 | 83 | 24 | 1992 | 0.41 | 33.76 |
| H10/H8 | 9 | 21 | 12 | 252 | 1.33 | 28.00 |
| H9/H9 | 17 | 30 | 13 | 390 | 0.76 | 22.94 |
| H4/H4 | 85 | 100 | 15 | 1500 | 0.18 | 17.65 |
| H8/H10 | 33 | 39 | 6 | 234 | 0.18 | 7.09 |
| H11/H11 | 24 | 27 | 3 | 81 | 0.13 | 3.38 |
| PS/PO | 5 | 5 | 0 | 0 | 0.00 | 0.00 |
| PO/PS | 5 | 5 | 0 | 0 | 0.00 | 0.00 |

In the foregoing Table 3, where there are two designations for the backbone chemistry, the first one indicates the chemistry of strands 153 and the second the chemistry of strand 154. Fully phosphodiester bonds are designated “PO,” fully phosphorothioate backbones are designated “PS.” Hybrid backbones are designated with an “H” followed by the number of phosphorothioate backbone linkages, starting from the 3′ end. Thus, H3 means that the three most 3′ linkages are phosphorothioate and the rest of the backbone linkages are phosphodiester. If only one designation is shown (such as just PO), both strands have the same backbone chemistry.

The data set forth in Table 3 indicate that if either strand is fully phosphothioated (e.g. PS/PO or PO.PS) then the decoy has a high affinity for both p65/p50 and p50/p50, and therefore lacks the specificity desired. Generally, although not always, a higher number of phosphorothioate linkages resulted in reduced specificity. Generally, hybrid strands with more than 8 phosphorothioate linkages lacked specificity, whereas those with fewer than 7 retained acceptable affinity and specificity. However, H4/H4 has extremely low affinity, while H3/H3 and H5/H5 were both in the acceptable range. H11 /PO and PO/H11 has good affinity and specificity. Based on half-life, specificity and affinity, H3/H3, H5/H5, H6/H6, and H7/H7 were identified as the optimal backbone for the 153/154 decoy. Optimal backbone chemistries for other decoys can be tested and determined in an analogous manner.

Specificity Relative to Other Transcription Factors

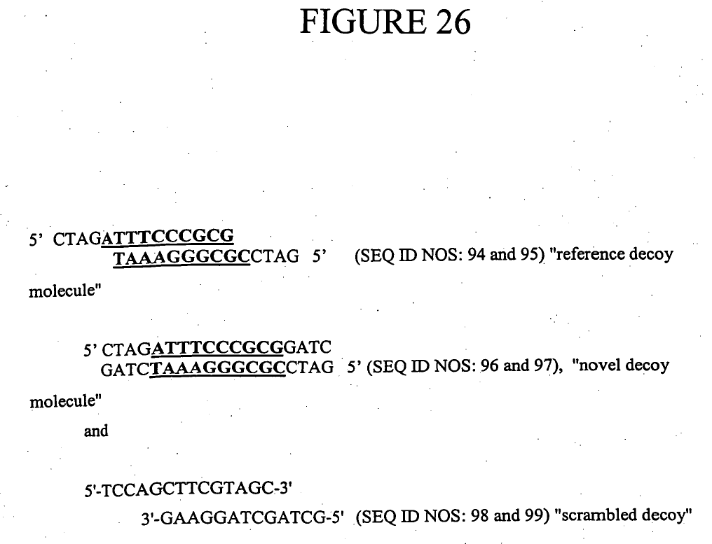

Decoy molecules must also specifically block only the target transcription factor and not non-specificially bind and block unrelated transcription factors. It has also been established that it is possible to design NF-κB decoy molecules which do not exhibit any non-specific effects on unrelated promoters using EMSA. Specifically, using radiolabeled oligonucleotide probes corresponding to the promoter sequences for the ubiquitous transcription factor Oct-1, it demonstrated that 153/154 (wherein “154” designates the reverse sequence corresponding to the sequence “153”) PO and H3 NF-κB decoy did not show any binding affinity for the promoter (FIG. 25). This is important because any non-specific effects of an oligonucleotide to other important proteins in the cell could result in unwanted toxicity of the decoy for the treatment individual.

Half-life

Native DNA is subject to rapid degradation inside a cell, primarily through the action of 3′ exonucleases, but also as a result of endonuclease attack. Therefore, when oligonucleotide decoys are designed, they are modified to enhance their stability. Replacing one of the non-bridging oxygen atoms of the intemucleotide linkage with a sulfur group, creating what is referred to as a phosphorothioate oligodeoxynucleotide, has been highly successful. The molecules are relatively nuclease resistant; however, they have been shown to exhibit non-specific protein binding relative to 3′-terminally modified and unmodified oligonucleotide decoys (Brown et al., J. Biol. Chem. 269:26801-5 (1994)). Therefore, a set of experiments were performed to determine how many sulfurs were required at the 3′- or 5′-end, or at an internal site to provide nuclease resistance to the oligonucleotide decoys herein, while maintaining specificity.

Binding specificity was assessed by the gel shift assay described above. 3′-exonuclease resistance was assessed using a standard snake venom assay (Cummins et al., Nucleic Acids Res. 23:2019-24 (1995)). To assess the resistance of the decoys to more relevant mammalian nuclease activity, as assay was adapted in which cytoplasmic and nuclear extracts were prepared from activated macrophages. (Hoke et al., Nucl. Acids Res. 19(20):5743-8 (1991)). The activity of the extracts was confirmed with positive controls in each assay. It was determined that capping the 3′-ends of each strand of the decoy with a few sulfur groups was sufficient to protect it from nuclease degradation.

Together these data indicate that for a p50/p65-selective NF-κB decoy 3-5 sulfurs at the 3′ ends of a 19-mer oligonucleotide duplex are sufficient to protect the decoy from nuclease degradation. Additionally, it was able to maintain specific subunit binding within the transcription factor family as well as lack of binding to irrelevant transcription factors. These data demonstrate that the present invention provides methods and means for designing specific and long-lasting oligonucleotide decoys targeting transcription factors, in particular NF-κB.

NF-κB Decoy Molecules Comprising a Nuclear Localization Signal

In order to determine the ability of a nuclear localization signal (NLS) containing peptide to improve the entry of an oligonucleotide decoy into the nucleus, a peptide with the NLS sequence based on the simian virus 40 large tumor antigen (PKKKRKVEDPYC) (SEQ ID NO78) was synthesized by Sigma Genosys and conjugated to the NF-κB 153 H3 oligonucleotide as follows. Briefly, 6.5 nmols of oligonucleotide was first incubated with 40-fold molar excess of the linker Sulfo-SMCC (Pierce) at room temperature for 2 hours. After removal of excess linker from the reaction by a NAP-10 column (Pharmacia Biotech), the activated oligonucleotide was incubated with 5-fold molar excess of the NLS peptide at room temperature overnight. To assess the percentage of oligonucleotide successfully conjugated to the NLS peptide, the reaction was analyzed by loading 1 μl onto a 20% PAGE gel (non-denaturing). The gel was stained with SYBR Gold (Molecular Probes) and visualized on a Typhoon Phosphorimager (Amersham). The concentration of the NLS-peptide conjugated single strand 153 H3 was determined by OD absorbance. The conjugate was then annealed to its complemetary strand 154 H3 (in equal molar amounts) containing a biotin molecule at its 5′ end. The presence of the biotin molecule on the now double stranded NLS decoy was to enable visualization (via streptavidin) of the localization through the use of microscopy.

### Example 13

### E2F Decoy Molecules

1. Synthesis of Oligonucleotide Decoys

The double-stranded oligonucleotide decoy molecules shown in FIG. 26 have been synthesized using an automated DNA synthesizer (Model 380B; Applied Biosystems, Inc., Foster City, Calif.). The decoys were purified by colunm chromatography, lyophilized, and dissolved in culture medium. Concentrations of each decoy were determined spectophotometrically.

The double-stranded oligonucleotide molecule represented by SEQ ID NOS: 94 and 95 (the “reference decoy molecule”) is a known decoy, currently in clinical development. The double-stranded oligonucleotide decoy represented by SEQ ID NOS: 96 and 97 (the “novel decoy molecule”) is a variant with significantly improved properties, while the “scrambled decoy” represented by SEQ ID NOS: 99 and 100 is used as a negative control.

The Tm of the novel decoy molecule is 55° C., significantly higher than the 42.3° C. Tm of the reference molecule. As a result, the novel decoy molecule is expected to be far more stable in vivo that the reference decoy.

2. Competitive Gel Mobility-Shift Assay

The difference in the ability of the novel decoy molecule to compete with the reference decoy and the negative control (scrambled decoy) to compete for E2F binding to a labeled probe containing an E2F consensus binding site was tested in vitro in LPS-stimulated THP-1 cells. essentially following the protocol described in Morishita et al., Proc. Natl. Acad. Sci. USA 92:5855-5859 (1995). Nuclear extract was prepared from vascular smooth muscle cells (VSMCs) as described in Horiuchi et al., J. Biol. Chem. 266:16247-16254 (1991). A gel-shift mobility assay was performed as follows:

A double-stranded oligonucleotide containing the E2F binding site (5′ CTAGATTTCCCGCGGATC 3′) (SEQ ID NO: 3) was end-labeled with γ³²P-ATP using T4 Polynucleotide Kinase (Promega). Five pg of a nuclear extract prepared from LPS stimulated THP-1 cells was incubated with 50 fmol of radiolabeled probe in the presence or absence of competing novel decoy molecule, the reference decoy or the negative control (scrambled decoy). The incubations were carried out at room temperature for 30 minutes in a 20 μl reaction volume composed of 10 mM Tris PH7.4, 40 mM KCL, 1 mM DTT, 0.1 mM EDTA, 8% glycerol, 0.05% NP-40 and 0.5μg Poly-dIdC. The reactions were loaded onto a 6% polyacrylamide gel, subjected to electrophoresis and dried. The dried gels were imaged and quantitated using a Typhoon 8600 PhosphorImager (Amersham) and ImageQuant software. The identity of the E2F proteins contained in complexes bound to the radiolabeled oligonucleotide probe were identified by pre-incubating the reactions for 5 minutes with individual antibodies specific for each member of the E2F family prior to the addition of the radiolabeled probe. Antibodies against E2F1(sc-193x, sc-251x), E2F2 (sc-633x), E2F3 (sc-878x, sc-879x), E2F4 (sc-866x), E2F5 (sc-999x), p107 (sc-318x) and cyclinA (sc-239x) were purchased from Santa Cruz Biotechnology.

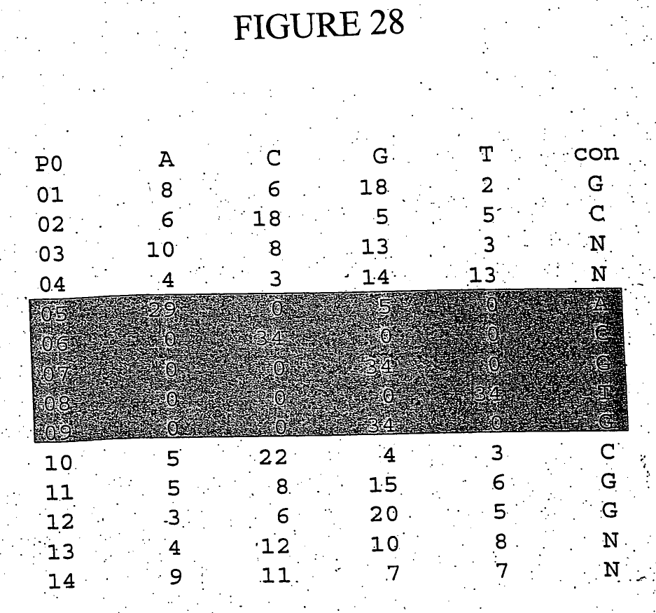

As shown in FIG. 27, the novel decoy molecule was able to compete with binding of a labeled probe with E2F in the smooth muscle extracts by greater than 60% at 10-fold molar excess (compared to 7% blockade by the reference decoy), and by 90% at 40-fold molar excess (compared to only 40% by the reference decoy). Thus, the novel decoy molecule is approximately a magnitude netter competitor than the reference decoy molecule of the prior art.

### Example 14

### HIF-1 Decoy Molecules Design

The HIF-1 binding DNA consensus sequences were selected from publications of HIF-1 related DNA-protein interactions, and were chosen from the sequence set summarized in BioBase TRANSFAC (version 7.2) database. Their corresponding regulatory region localizations have been confirmed and the extended flanking genomic DNA sequences retrieved from the most updated genome database (see Table 4) (for human, version July 2003; for mouse, version February, 2003; for rat, version June, 2003).

TABLE 4

Experimentally identified HIF-1 binding sites
and corresponding flanking sequences.

| | | SEQ |
|---|---|---|
| | | ID |
| Genes | Sequences | NO: |
| ADM_825 | GTGTGCTCCCAGTCAGTCAATCCTCACGTTTATGAT | 166 |
| | GGATGAATGAAGGCAG | |
| EDN1 | TTGTGTTATTAGTCACCAACAGGCAACGTGCAGCCG | 167 |
| | GAGATAAGGCCAG | |
| HMOX1_2 | ATCCCCCCGCCCACAGAGAGGACGTGCCACGCCAGC | 168 |
| | ACGTCCGCTCTCCTTGCCAG | |
| ADM_1 | TGTGCTCCCAGTCAGTCAATCCTCACGTTTATGATG | 168 |
| | GATGAATGAAGGCAGTCAGGT | |
| ADM_1203 | GTGATGAAAGAGCACAAACGGGTGACAAACGTGTCT | 170 |
| | AGCGTGATTCATCATGAACAGGCACA | |
| ADM_863 | TGCTTGGTAAACTGTAAAATGATTAGCATACGTGAA | 171 |
| | GCGTTAGTGTGCTCCCTGGCA | |
| Adra1b | GAGCGAGCCGCTGGGTGCAGGCAGGCGACGTGCTGC | 172 |
| | CGGGCTAGGCTGCCCGGGGGAGATGA | |
| ALDA_1 | GTGGTCCGAGTCACGTCCGAGGGGG | 173 |
| ALDA_2 | CTTCACGTGCGGGGACCAGGGACCGT | 174 |
| ENO1_1 | CGCAGGCGCAGGCGGCGCACGTGGCC | 175 |
| ENO1_364 | GAGTGCGTGCGGGACTCGGAGTACGTGACGGAGCCC | 176 |
| | CGAGCTCTCATGCC | |
| ENO1_383 | GGGGCCCCAGAGCGACGCTGAGTGCGTGCGGGACTC | 177 |
| | GGAGTACGTGACGGAGC | |
| ENO1_409 | GCAAGGTCGAGGGCCGGACGTGGGGCCCCAGAGCGA | 178 |
| | CGCTGAGTGCGTGCGG | |
| EPO_2 | GGGGCGTGAGCGGGGCTGCTGCAGACGTGCGTGTGG | 179 |
| | GTCATGGGGGCTGCTC | |
| EPO_1 | GCCCTACGTGCTGTCTCACACAGCCTGTCTGACCTC | 180 |
| | TCGACCT | |
| ET_1 | CTCCGGCTGCACGTTGCCTG | 181 |
| FLT1_1 | ATGGAGACATAATTGAGGAACAACGTGGAATTAGTG | 182 |
| | TCATAGCAAATGATCTAGG | |
| Hmox1 | GAGCGGACGTGCTGGCGTGGCACGTCCTCTC | 183 |
| LDH1(A)_1 | GACGCCCGCCCCCGGCCCAGCCTACACGTGGGTTCC | 184 |
| | CGCACGTCCGCTGGG | |
| LDH1(A)_2 | CGTCAGAGTGGGAGCCCAGCGGACGTGCGGGAACCC | 185 |
| | ACGTGTAGGCTGGGC | |
| Nos2 | GTGACTACGTGCTGCCTAGGGGCCACTGCC | 186 |
| PAI-1 | CCTGAATGCTCTTACACACGTACACACACAGAGCA | 187 |
| | GC | |
| PFKL | CCGGGTAGCTGGCGTACGTGCTGCAG | 188 |
| PGK1_1 | CCTTGCGGTTCGCGGCGTGCCGGACGTGACAAACGG | 189 |
| | AAGCCGCACGTCTCACTA | |
| PGK1_2 | CCTTGCGGTTCGCGGCGTGCCGGACGTGACAAACGG | 190 |
| | AAGCCGCACGTCTCACTA | |
| PPARA | CTGCCAGTGCACGTCAGTGG | 191 |
| RTP801 | GCCCGGCCGCTGTCACCGGGCAGGAGAGAACGTTGC | 192 |
| | TTACGTGCGCCCGGAGTCCATTGGCCAAGGCGGGCC | |
| Slc2a1_p1 | AAGGCCCTGGGTCCACAGGCGTGCCGTCTGACACGC | 193 |
| | ATCAGGCAGGCACTC | |
| Slc2a1_rat | CCATTTCTAGGGCCTTGGGTCCACAGGCGTGCTGGC | 194 |
| | TGACACGCATCAGGCCG | |
| TF_1 | TTCCTGCACGTACACACAAAGCGCACGTATTTC | 195 |
| TFREC | TCAGAGCACCTCGCGAGCGTACGTGCCTCAGGAAGT | 196 |
| | GACGCACAGCCCCCTGGGGGCCGG | |
| VEGF | GGGTTTTGCCAGACTCCACAGTGCATACGTGGGCTC | 197 |
| | CAACAGGTCCTCTTCCCTCCCAGTCACTG | |

Construction of Matrix of Core Consensus Binding Sites

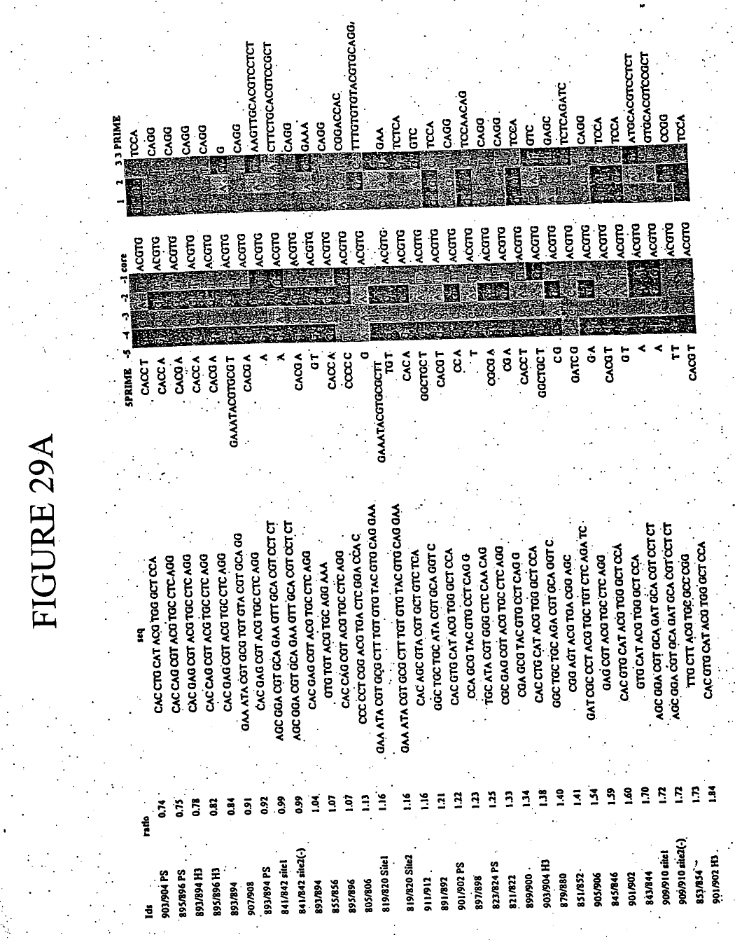

To define the base-composition near the core binding sites of HIF-1, the core binding sites based on the above available HIF-1 core binding sequences were computationally aligned. Based on the alignment, a table or “matrix” was created that computationally describes the base composition for both the core and the immediate-flanking regions (see FIG. 28). FIG. 28 statistically suggests the probability that a given base will be found at a given position.

Analysis of Crystal Structure of HIF-1 Binding Motif

HIF-1 is in a family of basic helix-loop-helix (bHLH) DNA binding proteins. The amino acids located from position 30 to position 70 (out of total 826 for the HIF-1a subunit) are responsible for the DNA recognition and binding affinity. While there is no crystal structure of the DNA binding motif for HIF-1, the available structural information of other bHLH members that share a similar DNA binding motifs provides useful structural information (Michel et al., Theor Chem Acc. 101:51-56 (1999); Michel et al., J. Biomolecular Structure & Dynamics 18:169-179 (2000); Michel et al., Biochimica et Biophysica Acta 1578:73-83 (2002)). These studies suggested the importance of several residues located in the binding motif of HIF-1. The central core ACGTG (SEQ ID NO: 165) is essential for binding of the HIF-1 complex (HIF-1α and ARNT), and the base-composition immediatly 5-prime upstream from the core is also very important for the specificity and affinity of HIF-i binding. DNA-footprint studies also suggested that the 5-prime upstream region could be important for HIF-1 induced gene expression. Therefore, candidate decoys with varying sequences and lengths of the 5′ flank were designed and tested.

Sequences of Initial HIF-1a Decoys

Based on the knowledge from published HIF-1 binding studies, from available HIF-1 core binding sequences, from the computational core binding matrix, from the model of crystal structures about bHLH family, and from specific bioinformatics approaches (to exclude the decoy that may binding to other transcription factors), a set of decoys were generated for initial screening (see Table 5). These decoys include a “mutation decoy”, “scramble decoys”, decoys with different length at their 5′ or 3′ end, and decoys with alternative base composition at or flank the core region.

TABLE 5
Initial Sequences for Screening

| | | SEQ |
|---|---|---|
| | | ID |
| # | Sequences | NO. |
| 01 | GCC CTA CGT GCT GTC TCA | 198 |
| 02 | TGA GAC AGC ACG TAG GGC | 199 |
| 03 | CTG TCC TCC GAC TGC ATG | 200 |
| 04 | CAT GCA GTC GGA GGA CAG | 201 |
| 05 | CCC CCT CGG ACG TGA CTC | 202 |
| | GGA CCA C | |
| 06 | G TGG TCC GAG TCA CGT CCG | 203 |
| | AGG GGG | |
| 07 | TCT GTA CGT GAC CAC ACT | 204 |
| | CAC CTC | |
| 08 | GAG GTG AGT GTG GTC ACG | 205 |
| | TAC AGA | |
| 09 | ACG GCC GGA CGT GGG GCC | 206 |
| | CC | |
| 10 | GG GGC CCC ACG TCC GGC | 207 |
| | CCT | |
| 11 | ACG CTG AGT GCG TGC GGG | 208 |
| | AC | |
| 12 | GT CCC GCA CGC ACT CAG CGT | 209 |
| 13 | GCC CTA CGT GCT GTC TCA | 210 |
| | CAC AGC | |
| 14 | GCT GTG TGA GAC AGC ACG | 211 |
| | TAG GGC | |
| 15 | GTG AGA CGT GCG GCT TCC | 212 |
| | GTT TG | |
| 16 | CA AAC GGA AGC CGC ACG | 213 |
| | TCT CAC | |
| 17 | CTG CCG ACG TGC GCT CCG | 214 |
| | GAG | |
| 18 | CTC CGG AGC GCA CGT CGG | 215 |
| | CAG | |
| 19 | GAA ATA CGT GCG CTT TGT | 216 |
| | GTG TAC GTG CAG GAA | |
| 20 | TTC CTG CAG GTA CAC ACA | 217 |
| | AAG CGC ACG TAT TTC | |
| 21 | CGC GAG CGT ACG TGC CTC | 218 |
| | AGG | |
| 22 | CGT GAG GCA CGT ACG CTC | 219 |
| | GCC | |
| 23 | TGC ATA CGT GGG CTC CAA | 220 |
| | CAG | |
| 24 | CTG TTG GAG CCC ACG TAT | 221 |
| | GCA | |
| 25 | AGG AGA CGT GCG AGA A | 222 |
| 26 | T TCT CGC ACG TCT CCT | 223 |
| 27 | AGG TTA CGT GCG GAC A | 224 |
| 28 | T GTC CGC ACG TAA CCT | 225 |
| 29 | AGG AGA CGT GCT GCC T | 226 |
| 30 | A GGC AGC ACG TCT CCT | 227 |
| 31 | TCC AAT ACG TGC AGT ACT | 228 |
| 32 | AGT ACT GCA CGT ATT GGA | 229 |
| 33 | TCC AAT GCG TGC AGT ACT | 230 |
| 34 | AGT ACT GCA CGC ATT GGA | 231 |
| 35 | GGC CAG ACG TGC CAC CGG | 232 |
| 36 | CCG GTG GCA CGT CTG GCC | 233 |
| 37 | AGG CAA CGT GCA GCC G | 234 |
| 38 | C GGC TGC ACG TTG CCT | 235 |
| 39 | AGG CAA TAC GCA GCC G | 236 |
| 40 | C GGC TGC GTA TTG CCT | 237 |
| 41 | AGC GGA CGT GCA GAA GTT | 238 |
| | GCA CGT CCT CT | |
| 42 | AG AGG ACG TGC AAC TTC | 239 |
| | TGC ACG TCC GCT | |
| 43 | GTG CAT ACG TGG GCT CCA | 240 |
| 44 | TGG AGC CCA CGT ATG CAC | 241 |
| 45 | GAG CGT ACG TGC CTC AGG | 242 |
| 46 | CCT GAG GCA CGT ACG CTC | 243 |
| 47 | GGA ACA ACG TGG AAT TAG | 244 |
| 48 | CTA ATT CCA CGT TGT TCC | 245 |
| 49 | GCC TAC ACG TGG GTT CCC | 246 |
| 50 | GGG AAC CCA CGT GTA GGC | 247 |
| 51 | CGG AGT ACG TGA CGG AGC | 248 |
| 52 | GCT CCG TCA CGT ACT CCG | 249 |
| 53 | TTG CTT ACG TGC GCC CGG | 250 |
| 54 | CCG GGC GCA CGT AAG CAA | 251 |
| 55 | GTG TGT ACG TGC AGG AAA | 252 |
| 56 | TTT CCT GCA CGT ACA CAC | 253 |
| 57 | GCG GAC GTG CGG GAA CCC | 254 |
| | ACG TGT AGG | |
| 58 | CCT ACA CGT GGG TTC CCG | 255 |
| | CAC GTC CGC | |
| 59 | ACC GTA CGT GCT GAT C | 256 |
| 60 | G ATC AGC ACG TAC GGT | 257 |
| 61 | CTA ATA CGT GCC GCT G | 258 |
| 62 | C AGC GGC ACG TAT TAG | 259 |
| 63 | AGC AGA CGT GCA GGA T | 260 |
| 64 | A TCC TGC ACG TCT GCT | 261 |
| 65 | AGC AGA CGT GCA GGC A | 262 |
| 66 | T GCC TGC ACG TCT GCT | 263 |
| 67 | TCC GTA CGT GCT GCA C | 264 |
| 68 | G TGC AGC ACG TAC GGA | 265 |
| 69 | AGC AGA CGT GCA GGG T | 266 |
| 70 | A CCC TGC ACG TCT GCT | 267 |
| 71 | ACC GTA CGT GCT GCC A | 268 |
| 72 | T GGC AGC ACG TAC GGT | 269 |
| 73 | TCC GTA CGT GCT GCG T | 270 |
| 74 | A CGC AGC ACG TAC GGA | 271 |
| 75 | TGC AGA CGT GCA GGT C | 272 |
| 76 | G ACC TGC ACG TCT GCA | 273 |
| 77 | ACC GTA CGT GCT GCT A | 274 |
| 78 | T AGC AGC ACG TAC GGT | 275 |
| 79 | GGC TGC TGC AGA CGT GCA | 276 |
| | GGT C | |
| 80 | G ACC TGC ACG TCT GCA GCA | 277 |
| | GCC | |
| 81 | GGC TGC AGG AGA CGT GGA | 278 |
| | GAA | |
| 82 | TTC TCC ACG TCT CCT GCA | 279 |
| | GCC | |
| 83 | AGA AGA CGT GCA GGA T | 280 |
| 84 | A TCC TGC ACG TCT TCT | 281 |
| 85 | TAC AGA CGT GCA GGT C | 282 |
| 86 | G ACC TGC ACG TCT GTA | 283 |
| 87 | GGC TGC ACC GTA CGT GCT | 284 |
| | GAT C | |
| 88 | G ATC AGC ACG TAC GGT GCA | 285 |
| | GCC | |
| 89 | TGC ATA CGT GCA GGT C | 286 |
| 90 | G ACC TGC ACG TAT GCA | 287 |
| 91 | GGC TGC TGC ATA CGT GCA | 288 |
| | GGT C | |
| 92 | G ACC TGC ACG TAT GCA GCA | 289 |
| | GCC | |
| 95 | CAC CAG CGT ACG TGC CTC | 340 |
| | *A*G*G | |
| 96 | CCT GAG GCA CGT ACG CTG | 341 |
| | *G*T*G | |

The sequences listed next to each other in the foregoing Table 5 (e.g. 801/802; 803/804, etc.) are complementary, and form the two strands of one double-stranded oligonucleotide decoy. For example, the HIF decoy molecule, HIF-1 decoy 895:896H3, has a upper strand- CA.C CA.G CGT ACG TGC CTC *A*G*G (SEQ ID NO: 340) and a complementary strand- CCT GAG GCA. CGT ACG CTG *G*T*G (SEQ ID NO: 341).

The Initial Screening Using TransAM Kit

To assess the relative affinities of oligonucleotides for a HIF-1α containing complex, the HIF-1 TransAM assay (Active Motif, Catalog # 47096) was utilized. The assay was performed according to manufacturer's instructions. Briefly, a double-stranded oligonucletide containing the hypoxia response element (HRE) was immobilized on a 96-well plate. A nuclear extract containing HIF-1 alpha complexes was incubated and allowed to bind to the immobilized oligonucleotide. The unbound material was washed away and the bound HIF-1α detected using an antibody that specificially recognizes HIF-1α. The anti-HIF-1α antibody was detected by a secondary antibody labeled with horseradish peroxidase (HRP), and the amount of HRP in each well was measured using a colorimetric substrate reaction and read using a microplate spectrophotometer.

The ability of candidate decoy molecules to compete for binding of HIF-1α to the HRE element immobilized on the plate were measured and compared to reveal relative binding affinities. Candidate decoys were added in increasing molar ratios (relative to the amount of oligo immobilized on the plate) to compete for binding to the HIF-1α containing complexes. The amounts of decoys added to the assay included 0.625, 1.25, 2.5, 5, 10 and 20 picomoles. A well containing a competing decoy able to bind HIF-1α with high affinity would give a lower absorbance reading as compared to a decoy with low affinity for HIF-1α. All potential decoys were then compared and ranked in order to assess their relative binding affinities.

The Analysis of TransAM Result

The screen was conducted using different decoy concentrations. For each UV absorbance reading, normalization was done by calculating the ratio of absorbance readings of sample vs. wild type control. The results are summarized in Table 6. The bigger ratio represents less competition of binding with HIF-1α when compared with wild-type control. The smaller (smaller than 1.0 or close to 1.0) ratios represent better binding or better competition.

TABLE 6

| | | | SEQ |
|---|---|---|---|
| ID | RATIO | FORWARD SEQUENCES | ID |
| 801/802 | 1.83 | GCC CTA CGT GCT GTC TCA | 290 |
| 803/804_scramble | 2.19 | CTG TCC TCC GAC TGC ATG | 291 |
| 805/806 | 1.09 | CCC CCT CGG ACG TGA CTC GGA CCA C | 292 |
| 807/808 | 2.38 | TCT GTA CGT GAC CAC ACT CAC CTC | 293 |
| 809/810 | 2.60 | AGG GCC GGA CGT GGG GCC CC | 294 |
| 811/812 | 2.37 | ACG CTG AGT GCG TGC GGG AC | 295 |
| 813/814 | 2.88 | GCC CTA CGT GCT GTC TCA CAC AGC | 296 |
| 815/816 | 2.58 | GTG AGA CGT GCG GCT TCC GTT TG | 297 |
| 817/818 | 3.40 | CTG CCG ACG TGC GCT CCG GAG | 298 |
| 819/820_double | 0.88 | GAA ATA CGT GCG CTT TGT GTG TAC GTG | 299 |
| | | CAG GAA | |
| 821/822 | 1.26 | CGC GAG CGT ACG TGC CTC AGG | 300 |
| 823/824 | 2.76 | TGC ATA CGT GGG CTC CAA CAG | 301 |
| 825/826 | 2.91 | AGG AGA CGT GCG AGA A | 302 |
| 827/828 | 2.62 | AGG TTA CGT GCG GAC A | 303 |
| 829/830 | 2.81 | AGG AGA CGT GCT GCC T | 304 |
| 831/832 | 2.26 | TCC AAT ACG TGC AGT ACT | 305 |
| 833/834 | 2.48 | TCC AAT GCG TGC AGT ACT | 306 |
| 835/836 | 2.51 | GGC CAG ACG TGC CAC CGG | 307 |
| 837/838 | 2.46 | AGG CAA CGT GDA GCC G | 308 |
| 839/840_mutation | 2.53 | AGG CAA TAC GCA GCC G | 309 |
| 841/842_double | 0.95 | AGC GGA CGT GCA GAA GTT GCA CGT CCT | 310 |
| | | CT | |
| 843/844 | 1.81 | GTG CAT ACG TGG GCT CCA | 311 |
| 845/846 | 1.57 | GAG CGT ACG TGC CTC AGG | 312 |
| 847/848 | 2.23 | GGA ACA ACG TGG AAT TAG | 313 |
| 849/850 | 1.96 | GCC TAC ACG TGG GTT CCC | 314 |
| 851/852 | 1.08 | CGG AGT ACG TGA CGG AGC | 315 |
| 853/854 | 1.62 | TTG CTT ACG TGC GCC CGG | 316 |
| 855/856 | 1.21 | GTG TGT ACG TGC AGG AAA | 317 |
| 857/858_double | 2.30 | GCG GAC GTG CGG GAA CCC ACG TGT AGG | 318 |
| 859/860 | 1.85 | ACC GTA CGT GCT GAT C | 319 |
| 861/862 | 2.50 | CTA ATA CGT GCC GCT G | 320 |
| 863/864 | 2.02 | AGC AGA CGT GCA GGA T | 321 |
| 865/866 | 2.01 | AGC AGA CGT GCA GGC A | 322 |
| 867/868 | 2.27 | TCC GTA CGT GCT GCA C | 323 |
| 869/870 | 1.93 | AGC AGA CGT GCA GGG T | 324 |
| 871/872 | 1.87 | ACC GTA CGT GCT GCC A | 325 |
| 873/874 | 2.04 | TCC GTA CGT GCT GCG T | 326 |
| 875/876 | 2.11 | TGC AGA CGT GCA GGT C | 327 |
| 877/878 | 2.29 | ACC GTA CGT GCT GCT A | 328 |
| 879/880 | 1.98 | GGC TGC TGC AGA CGT GCA GGT C | 329 |
| 881/882 | 3.39 | GGC TGC AGG AGA CGT GGA GAA | 330 |
| 883/884 | 3.76 | AGA AGA CGT GCA GGA T | 331 |
| 885/886 | 4.02 | TAC AGA CGT GCA GGT C | 332 |
| 887/888 | 1.94 | GGC TGC ACC GTA CGT GCT GAT C | 333 |
| 889/890 | 2.90 | TGC ATA CGT GCA GGT C | 334 |
| 891/892 | 1.60 | GGC TGC TGC ATA CGT GCA GGT C | 335 |

(1) Comparison of the immediate 5′ sequences suggests that the base composition of GCalif.G or GGAG or GCalif.T or CCCT or CCGT could lead to poor competition (e.g., bigger ratio, compared with wild type decoy.

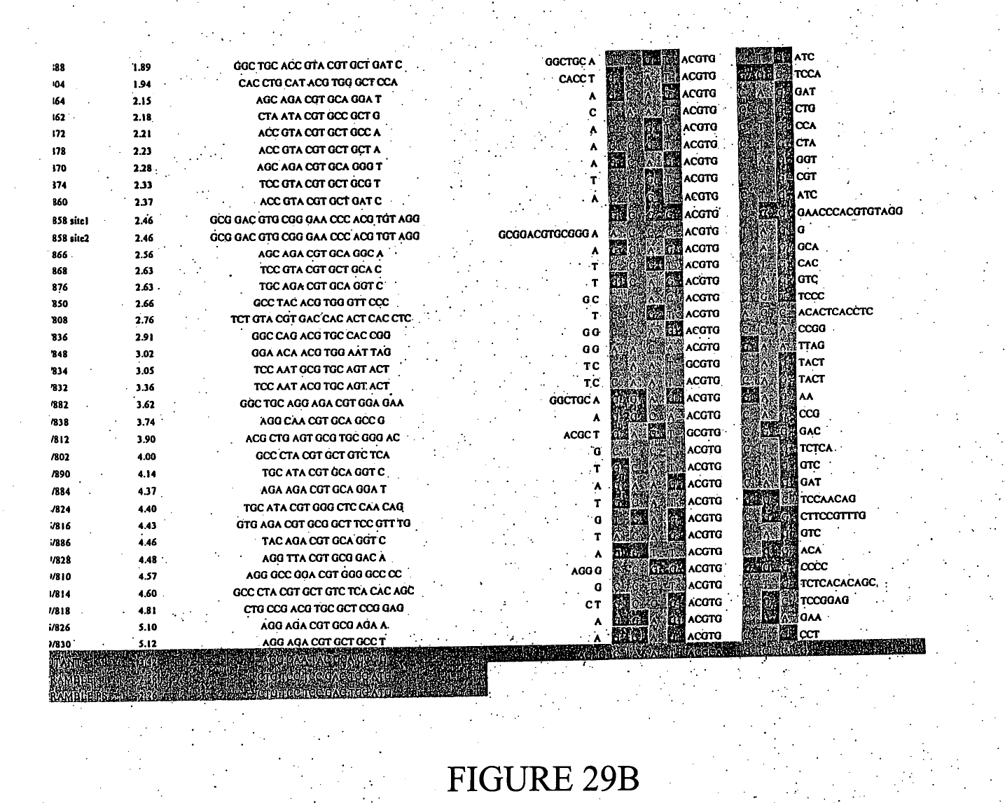

(2) If we sort the ratio, those decoys with better competition (e.g., smaller ratio) mostly share base “G” and base “T” at position “-4” and “-1” respectively (FIG. 29). The 4 bases immediately before core (ACGTG; SEQ ID NO: 336) will be more like “GCGT” (SEQ ID NO: 337) for the better competition decoys (FIG. 29). FIG. 29 also suggests that the combination of “G” at position “-4” with “G” at position “-1” does not favor the binding affinity, same to the combination of “A” at position “-3” and “A” at position “-2”, respectively.

Confirmation by EMSA

The HIF-1 gel shift assays (EMSA) were performed as follows. A double-stranded oligonucleotide containing a consensus HIF-1 binding site was end-labeled with γ³²P-ATP using T4 Polynucleotide Kinase (Promega). One microgram of a nuclear extract prepared from LPS stimulated THP-1 cells (human monocyte cell line) was incubated with 35 fmol of radiolabeled probe in the presence or absence of competing unlabeled HIF-1 double-stranded oligonucleotides (dsODN) or scrambled dsODN. The incubations were carried out at room temperature for 30 minutes in a 20 μl reaction volume composed of 10 mM Tris-HCl pH 8, 100 mM KCL, SmM MgC12, 2 mM DTT, 10% Glycerol, 0.1% NP-40, 0.025% BSA and 1 μg Poly-dIdC. The reactions were loaded onto a 6% polyacrylamide gel, subjected to electrophoresis and dried. The dried gels were imaged and quantitated using a Typhoon 8600 Phosphorlmager (Amersham) and ImageQuant software. The identity of the HIF-1 proteins contained in complexes bound to the radiolabeled oligonucleotide probe were identified by pre-incubating the reactions for 5 minutes with individual antibodies specific for each member of the HIF- 1 family prior to the addition of the radiolabeled probe.

The binding of selected decoys is confirmed by conventional EMSA method.

All references cited throughout the specification is hereby expressly incorporated by reference.

Although the present invention is illustrated with reference to certain specific embodiments, it is not so limited. Modifications and variations are possible without diverting from the idea of the invention, and will be apparent to those skilled in the art. All such modifications and variations are specifically within the scope herein.

What is claimed is:

1. A method for delivering a polynucleotide to a cell, said method comprising contacting a biological membrane with a formulation containing said polynucleotide, at least one penetration enhancer in a total concentration of about 0.3% to about 10% by weight, and an alcohol in a concentration of about 1% to about 60% by weight.

2. A method for delivering a polynucleotide to a cell, said method comprising contacting a biological membrane with an emulsion-based formulation containing said polynucleotide, at least one penetration enhancer in a total concentration of about 0.2% to about 10% by weight, and water wherein said penetration enhancer is selected from the group consisting of sodium laureth sulfate, N-lauroylsarcosine, sorbitan monolaurate 20 (Span 20) and isopropyl myristate.

3. The method of claim 1 or 2 wherein said polynucleotide is an oligonucleotide.

4. The method of claim 3 wherein said cell is that of a mammal.

5. The method of claim 4 wherein said mammal is human.

6. The method of claim 3 wherein the biological membrane is skin or a mucosal membrane.

7. The method of claim 3 wherein said penetration enhancer is an anionic surfactant.

8. The method of claim 7 wherein the anionic surfactant is s an alkyl sulfate or an alkyl ether sulfate.

9. The method of claim 8 wherein the anionic surfactant is sodium lauryl sulfate or sodium laureth sulfate.

10. The method of claim 3 wherein said penetration enhancer is N-lauroylsarcosine.

11. The method of claim 3 wherein said penetration enhancer is sorbitan monolaurate 20 (Span 20).

12. The method of claim 3 wherein said formulation comprises about 0.4% to about 10% by weight of said penetration enhancer.

13. The method of claim 3 wherein said formulation comprises about 0.8% by weight of said penetration enhancer.

14. The method of claim 13 wherein said penetration enhancer is sodium laureth sulfate.

15. The method of claim 3 wherein said formulation comprises about 0.6% by weight of said penetration enhancer.

16. The method of claim 15 wherein said penetration enhancer is N-lauroylsarcosine.

17. The method of claim 3 wherein said formulation comprises about 0.4% by weight of said penetration enhancer.

18. The method of claim 17 wherein said penetration enhancer is sorbitan monolaurate 20 (Span 20).

19. The method of claim 3 wherein said alcohol is ethanol.

20. The method of claim 3 wherein said formulation comprises about 1% to about 50% by weight of the alcohol.

21. The method of claim 3 wherein said formulation is an aqueous formulation.

22. The method of claim 21 wherein said aqueous formulation is an aqueous gel-based formulation.

23. The method of claim 22 wherein said aqueous gel-based formulation comprises about 0.8% by weight of sodium laureth sulfate.

24. The method of claim 23 wherein said aqueous gel-based formulation further comprises about 5% by weight of ethanol.

25. The method of claim 23 wherein said aqueous gel-based formulation further comprises about 10% by weight of ethanol.

26. The method of claim 23 wherein said aqueous gel-based formulation further comprises about 20% by weight of ethanol.

27. The method of claim 23 wherein said aqueous gel-based formulation further comprises about 49% by weight of ethanol.

28. The method of claim 3 wherein said formulation is a liposome-containing formulation.

29. The method of claim 28 wherein said liposome-containing formulation comprises about 0.8% by weight of sodium laureth sulfate.

30. The method of claim 28 wherein said liposome-containing formulation further comprises about 10% by weight of ethanol.

31. The method of claim 28 wherein said liposome-containing formulation further comprises about 5% by weight of ethanol.

32. The method of claim 28 wherein said liposome-containing formulation further comprises about 2.5% by weight of ethanol.

33. The method of claim 28 wherein said liposome-containing formulation comprises about 0.6% by weight of N-lauroylsarcosine.

34. The method of claim 33 wherein said liposome-containing formulation further comprises about 0.4% by weight of sorbitan monolaurate 20 (Span 20).

35. The method of claim 34 wherein said liposome-containing formulation further comprises about 5% by weight of ethanol.

36. The method of claim 3 wherein said emulsion-based formulation comprises about 0.8% by weight of said sodium laureth sulfate.

37. The method of claim 3 wherein said emulsion-based formulation comprises about 0.35% by weight of said sodium laureth sulfate.

38. The method of claim 37 wherein said emulsion-based formulation further comprises about 0.15% by weight of 1-phenyl piperazine.

39. The method of claim 3 wherein said emulsion-based formulation comprises about 0.6% by weight of N-lauroylsarcosin.

40. The method of claim 39 wherein said emulsion-based formulation further comprises about 0.4% by weight of sorbitan monolaurate 20 (Span 20).

41. The method of claim 3 wherein said emulsion-based formulation further comprises about 10% by weight of isopropyl myristate.

42. The method of claim 3 wherein said emulsion-based formulation further comprises HPMC 4000 cps, polyoxyl-40 stearate, glyceryl monostearate, methyl paraben and propyl paraben.

43. The method of claim 3 wherein said oligonucleotide is a double stranded oligodeoxynucleotide (dsODN) molecule.

44. The method of claim 43 wherein the first strand of the dsODN molecule is at least partially complementary to the second strand.

45. The method of claim 43 wherein the first strand of the dsODN molecule is fully complementary to the second strand.

46. The method of claim 43 wherein the dsODN molecule comprises at least one single-stranded overhang.

47. The method of claim 43 wherein the dsODN molecule comprises two oligodeoxynucleotide strands that are covalently attached to each other at either the 3′ or the 5′ end, or both, resulting in a dumbbell structure, or a circular molecule.

48. The method of claim 43 wherein the dsODN molecule has a phosphodiesterate backbone, a phosphorothioate backbone, or a mixed phophodiesterate-phosphorothioate backbone.

49. The method of claim 43 wherein said first and second strands of the dsODN molecule are connected to each other solely by Watson-Crick base pairing.

50. The method of claim 43 wherein the dsODN is at least 15 base pairs long.

51. The method of claim 43 wherein the dsODN molecule comprises a sequence that is capable of specific binding to a transcription factor.

52. The method of claim 51 wherein the transcription factor is selected from the group consisting of E2F, AP-1, AP-2, HIF-1 and NFκB.

53. The method of claim 52 wherein the dsODN molecule is capable of specific binding to an NFκB transcription factor.

54. The method of claim 52 wherein the dsODN molecule binds to said E2F transcription factor with a binding affinity that is at least about 10-fold of the binding affinity of said reference molecule.

55. The method of claim 52 wherein the dsODN molecule is capable of specific binding to an HIF-1 transcription factor.

56. A method of treating an inflammatory disease or condition, comprising administering to a mammalian subject an effective amount of a pharmaceutical composition comprising a formulation specified in any one of claims 1-55.

57. The method according to claim 56 wherein said oligonucleotide is a double stranded oligodeoxynucleotide (dsODN) molecule capable of specific binding to an NFκB transcription factor.

58. The method of claim 57 wherein the concentration of said dsODN molecule is about 0.1% to about 1.0% by weight of the total formulation.

59. The method of claim 57 wherein the concentration of said dsODN molecule is about 0.1% by weight of the total formulation.

60. The method of claim 57 wherein the concentration of said dsODN molecule is about 0.25% by weight of the total formulation.

61. The method of claims 57 wherein the concentration of said dsODN molecule is about 0.5% by weight of the total formulation.

62. The method of claim 57 wherein said oligonucleotide is a double stranded oligonucleotide (dsODN) molecule capable of specific binding to an HIF-1 transcription factor.

63. The method of claim 57 wherein the inflammatory disease or condition is skin inflammation or skin cancer.

64. The method of claim 63 wherein the disease or condition is associated with acute or chronic skin inflammation.

66. The method of claim 63 wherein the skin cancer is basal-cell carcinoma (BCC), squarnous-cell carcinoma (SCC), or melanoma.

67. The method of claim 57 wherein the disease or condition is selected from the group consisting of atopic dermatitis, contact dermatitis, seborrheic dermatitis, psoriasis, rosacea; eczema, acne, alopecia, wound healing and scar tissue.

68. The method of claim 66 wherein the condition is atopic dermatitis.

69. The method of claim 66 wherein the condition is psoriasis.

70. The method of claim 57 wherein the mammalian subject is a human.

71. A pharmaceutical composition comprising a formulation specified in any one of claims 1-55 for the treatment of an inflammatory disease or condition in a mammalian subject.

72. The pharmaceutical composition of claim 70 wherein the mammalian subject is a human.

73. A formulation comprising an oligonucleotide, at least one penetration enhancer in a total concentration of about 0.2% to about 10% by weight, and an alcohol in a concentration of about 1% to about 60% by weight.

74. The formulation of claim 72 wherein said oligonucleotide is a double-stranded oligodeoxynucleotide (dsODN) molecule.

75. The formulation of claim 73 which is an aqueous formulation.

76. The formulation of claim 73 which is an aqueous gel-based formulation.

77. The formulation of claim 73 which is a liposome-containing formulation.
